SEQUENCE LISTING (6 sequences)

SEQ ID NO: 1 - protein, 186 aa, synthetic construct
  REGION 1..186: note = Protein sequence of Gpe with PelB signal sequence
MKYLLPTAAAGLLLLAAQPAMAMNIFEMLRIDERLRLKIYKDTEGYYTIGIGHLLTKSPSLNAAKSELDKAIGRNCNGVITKDEAEKLFNQDVDAAVRGILRNAKLKPVYDSLDAVRRCALINMVFQMGETGVAGFTNSLRMLQQKRWDEAAVNLAKSIWYNQTPNRAKRVITTFRTGTWDAYKNL

SEQ ID NO: 2 - protein, 417 aa, Homo sapiens
VTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRAMWLSLFSSEESNLGANNYDDYRMDWLVPATCEPIQSVFFFSGDKYYRVNLRTRRVDTVDPPYPRSIAQYWLGCPAPGHL

SEQ ID NO: 3 - protein, 287 aa, Saccharomyces cerevisiae
MTYTTRQIGAKNTLEYKVYIEKDGKPVSAFHDIPLYADKENNIFNMVVEIPRWTNAKLEITKEETLNPIIQDTKKGKLRFVRNCFPHHGYIHNYGAFPQTWEDPNVSHPETKAVGDNDPIDVLEIGETIAYTGQVKQVKALGIMALLDEGETDWKVIAIDINDPLAPKLNDIEDVEKYFPGLLRATNEWFRIYKIPDGKPENQFAFSGEAKNKKYALDIIKETHDSWKQLIAGKSSDSKGIDLTNVTLPDTPTYSKAASDAIPPASLKADAPIDKSIDKWFFISGSV

SEQ ID NO: 4 - protein, 684 aa, Moloney Murine leukemia virus
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITENPDTSTLLIENSSPNSRLIN

SEQ ID NO: 5 - protein, 456 aa, Sus scrofa
MNLDIHCEQLSDARWTELLPLLQQYEVVRLDDCGLTEEHCKDIGSALRANPSLTELCLRTNELGDAGVHLVLQGLQSPTCKIQKLSLQNCSLTEAGCGVLPSTLRSLPTLRELHLSDNPLGDAGLRLLCEGLLDPQCHLEKLQLEYCRLTAASCEPLASVLRATRALKELTVSNNDIGEAGARVLGQGLADSACQLETLRLENCGLTPANCKDLCGIVASQASLRELDLGSNGLGDAGIAELCPGLLSPASRLKTLWLWECDITASGCRDLCRVLQAKETLKELSLAGNKLGDEGARLLCESLLQPGCQLESLWVKSCSLTAACCQHVSLMLTQNKHLLELQLSSNKLGDSGIQELCQALSQPGTTLRVLCLGDCEVTNSGCSSLASLLLANRSLRELDLSNNCVGDPGVLQLLGSLEQPGCALEQLVLYDTYWTEEVEDRLQALEGSKPGLRVIS

SEQ ID NO: 6 - protein, 309 aa, synthetic construct
  REGION 1..309: note = vp39 protein without 32 C-terminal amino acids
MDVVSLDKPFMYFEEIDNELDYEPESANEVAKKLPYQGQLKLLLGELFFLSKLQRHGILDGATVVYIGSAPGTHIRYLRDHFYNLGVIIKWMLIDGRHHDPILNGLRDVTLVTRFVDEEYLRSIKKQLHPSKIILISDVRSKRGGNEPSTADLLSNYALQNVMISILNPVASSLKWRCPFPDQWIKDFYIPHGNKMLQPFAPSYSAEMRLLSIYTGENMRLTRVTKSDAVNYEKKMYYLNKIVRNKVVVNFDYPNQEYDYFHMYFMLRTVYCNKTFPTTKAKVLFLQQSIFRFLNIPTTSTGHHHHHHG

(19) United States
(12) Patent Application Publication
(10) Pub. No.: US 2026/0297643 A1
(43) Pub. Date: Oct. 1, 2026

(54) Culture Method

(71) Applicant: Thermo Fisher Scientific Baltics UAB, Vilnius (LT)

(72) Inventors: Dmitrij Bugajev, Vilnius (LT); Kristina Pagarauskaite, Širvintos (LT); Juozas Šiurkus, Vilnius (LT)

(21) Appl. No.: 19/142,087

(22) PCT Filed: Dec. 19, 2023
(86) PCT No.: PCT/EP2023/086717

§ 371 Date: Jun. 20, 2025

(30) Foreign Application Priority Data
Dec. 23, 2022 (GB) 2219698.4
Aug. 14, 2023 (GB) 2312374.8
Aug. 14, 2023 (GB) 2312376.3
Aug. 14, 2023 (GB) 2312385.4
Aug. 14, 2023 (GB) 2312386.2
Aug. 14, 2023 (GB) 2312388.8
Aug. 14, 2023 (GB) 2312393.8

(51) Int. Cl. C12P 21/02; C07K 1/30; C07K 1/34; C07K 1/36; C12N 1/20; C12N 15/70; C12R 1/19

(52) U.S. Cl. CPC C12P 21/02; C07K 1/30; C07K 1/34; C07K 1/36; C12N 1/20; C12N 15/70; C12R 2001/19

(57) ABSTRACT

The invention provides a method for producing a clarified cell culture comprising a recombinant protein, wherein the method comprises: i) inoculating a bioreactor with a host cell to provide a cell culture, wherein said host cell is an Escherichia coli transformed with an expression system comprising: a) a nucleic acid encoding a recombinant protein operably linked to a first inducible promotor; and b) a nucleic acid encoding a T4 lysozyme protein in frame with a nucleic acid encoding PelB secretory signal peptide operably linked to a second inducible promotor, ii) fermenting the cell culture under conditions that: a) allow the expression of the recombinant protein; and b) allow permeabilization of the cell so as to form a spheroplast that secretes the recombinant protein; iii) clarifying the fermented cell culture comprising the secreted recombinant protein, wherein said clarifying comprises a primary clarification step using a first clarification filter having a pore size that provides a retention range of at least about 0.4 μm or more, wherein the clarification step does not comprise centrifugation.

This invention relates to a system for expressing recombinant proteins using E. coli.

### BACKGROUND

Escherichia coli is often the microorganism of choice for recombinant protein production. The main reasons for this popularity are high growth rates and expression levels, as well as the simple and inexpensive growth media required. However, E. coli is a poor secretor of proteins and intracellular protein production methods employing E. coli require cell disruption and the removal of cell debris. In contrast, extracellular production of recombinant proteins and enzymes greatly reduces the complexity of the bioprocess production method and improves the quality of the recombinant product, whilst also affording simplified protein detection and purification. Additionally, an environment free of cell-associated proteolytic degradation has been shown to provide an optimised environment for protein folding.

A variety of techniques may be employed to facilitate the preparation of intracellular proteins from E. coli. Typically, the initial steps in these techniques involve lysis or rupture of the bacterial cells, to disrupt the bacterial cell wall and allow release of the intracellular proteins into the extracellular milieu. Following this release, the desired proteins are purified from the extracts, typically by a series of chromatographic steps.

Several approaches have proven useful in accomplishing the release of intracellular proteins from bacterial cells. Included among these are the use of chemical lysis, physical methods of disruption, or a combination of chemical and physical approaches (Felix, H., Anal. Biochem. 120:211-234 (1982)).

Chemical methods of disruption of the bacterial cell wall that have proven useful include treatment of cells with organic solvents such as toluene (Putnam, S. L., and Koch, A. L., Anal. Biochem. 63:350-360 (1975); Laurent, S. J., and Vannier, F. S., Biochimie 59:747-750 (1977); Felix, H., Anal. Biochem. 120:211-234 (1982)), with chaotropes such as guanidine salts (Hettwer, D., and Wang, H., Biotechnol. Bioeng. 33:886-895 (1989)), with antibiotics such as polymyxin B (Schupp, J. M., et al., BioTechniques 19:18-20 (1995); Felix, H., Anal. Biochem. 120:211-234 (1982)), or with enzymes such as lysozyme or lysostaphin (McHenty, C. S., and Kornberg, A., J. Biol. Chem. 252(18):6478-6484 (1977); Cull, M., and McHenry, C. S., Meth. Enzymol. 182:147-153 (1990); Hughes, A. J., Jr., et al., J. Cell Biochem. Suppl. 016 (Part B):84 (1992); Sambrook, J., et al., in Molecular Cloning: A Laboratory Manual, 2nd ed., Cold Spring Harbor, N.Y.: Cold Spring Harbor Laboratory Press (1989), pp. 17-38; Ausubel, F. M., et al., in Current Protocols in Molecular Biology, New York: John Wiley & Sons (1993), pp. 4.4.1-4.47). The effects of these various chemical agents may be enhanced by concurrently treating the bacterial cells with detergents such as Triton X-100@, sodium dodecylsulfate (SDS) or Brij 35 (Laurent, S. J., and Vannier, F. S., Biochimie 59:747-750 (1977); Felix, H., Anal. Biochem. 120:211-234 (1982); Hettwer, D., and Wang, H., Biotechnol. Bioeng, 33:886-895 (1989); Cull, M., and McHenry, C. S., Meth. Enzymol. 182:147-153 (1990); Schupp, J. M., et al., BioTechniques 19:18-20 (1995)), or with proteins or protamines such as bovine serum albumin or spermidine (McHenry, C. H. and Komberg, A., J. Biol. Chem. 252(18): 6478-6484 (1977); Felix, H., Anal. Biochem. 120:211-234 (1982); Hughes, A. J., Jr., et al., J. Cell Biochem. Suppl. 0 16 (Part B):84 (1992)).

In addition to these various chemical treatments a number of physical methods of disruption have been used. These physical methods include osmotic shock, e.g., suspension of the cells in a hypotonic solution in the presence or absence of emulsifiers (Roberts, J. D., and Lieberman, M. W., Biochemistry 18:4499-4505 (1979); Felix, H., Anal. Biochem. 120:211-234 (1982)), drying (Mowshowitz, D. B., Anal. Biochem. 70:94-99 (1976)), bead agitation such as ball milling (Felix, H., Anal. Biochem. 120:211-234 (1982); Cull, M., and McHenry, C. S., Meth. Enzymol. 182:182:147-153 (1990)), temperature shock, e.g., freeze-thaw cycling (Lazzarini, R. A., and Johnson L. D., Nature New Biol. 243:17-20 (1975); Felix, H., Anal, Biochem. 120:211-234 (1982)), sonication (Amos, H., et al., J. Bacteriol. 94:232-240 (1967); Ausubel, F. M., et al., in Current Protocols in Molecular Biology, New York, John Wiley & Sons (1993), pp. 4.4.1-4.47) and pressure disruption, e.g., use of a French pressure cell (Ausubel, F. M., et al., in Current Protocols in Molecular Biology, New York, John Wiley & Sons (1993), pp. 16.8.6-16.8.8). Other approaches combine these chemical and physical methods of disruption, such as lysozyme treatment followed by sonication or pressure treatment, to maximize cell disruption and protein release (Ausubel, F. M., et al., in Current Protocols in Molecular Biology, New York, John Wiley & Sons (1993), pp. 4.4.1-4.47).

These disruption approaches have several advantages, including their ability to rapidly and completely disrupt the bacterial cell such that the release of intracellular proteins is maximized.

However, these methods possess distinct disadvantages as well. For example, the physical methods by definition involve shearing and fracturing of the bacterial cell walls and plasma membranes. These processes thus result in extracts containing large amounts of particulate matter, such as membrane or cell wall fragments, which must be removed from the extracts, typically by centrifugation, prior to purification of the recombinant proteins. This need for centrifugation limits the batch size capable of being processed in a single preparation to that of available centrifuge space; thus, large production-scale preparations are impracticable if not impossible. Furthermore, physical methods, and many chemical techniques, typically result in the release from the cells not only of the desired intracellular proteins, but also of undesired nucleic acids and membrane lipids (the latter particularly resulting when organic solvents are used). These undesirable cellular components also complicate the subsequent processes for purification of the desired proteins, as they increase the viscosity of the extracts (Sambrook, J., et al., in: Molecular Cloning: A Laboratory Manual, 2nd ed., Cold Spring Harbor, N.Y.: Cold Spring Harbor Laboratory Press (1989), pp 17-38; Cull, M., and McHenry, C. S., Meth. Enzymol. 182:147-153 (1990)), and bind with high avidity and affinity to nucleic acid-modifying proteins such as DNA polymerases, RNA polymerases and restriction enzymes.

Naturally occurring lytic and temperate bacteriophages have the ability to provoke host cell lysis through the expression of specific proteins during the lytic cycle. These lytic proteins have been identified and widely studied, in for example T4 and T7 bacteriophages. The lytic proteins include both holins and lyzozymes. Holins form stable and non-specific lesions in the cytoplasmic membrane that allow the lysozymes to gain access to the peptidoglycan layer. Lysozymes exhibit muralytic activities against the three different types of covalent bonds (glycosidic, amide, and peptide) of the peptidoglycan polymer of the cell wall. Together, the activity of holin and lysozyme breaks down the two cell membranes of gram-negative bacteria, thus causing cell lysis.

E. coli has been engineered to express T4 bacteriophage lytic proteins so as to provide programmed cell lysis in an attempt to improve the efficiency and economy of the downstream processing for recombinant protein production.(M. Morita, K. Asami, Y. Tanji, and H. Unno, “Programmed Escherichia coli Cell Lysis by Expression of Cloned T4 Phage Lysis Genes,” Biotechnology Progress, vol. 17, (no. 3), pp. 573-576, 2001).

Escherichia coli based constructs are the gold standard for recombinant protein production when post-translation modifications are unnecessary. The main reasons for the popularity of E. coli are high growth rates and expression levels, as well as simple and inexpensive growth media.

However, E. coli is not a perfect host because it normally does not secrete proteins into the extracellular medium. Thus, one problem associated with approaches that employ E. coli is that the cell disruption step becomes a limitation and not cost effective at the pilot or industrial scales when the goal is the implementation of closed and/or continuous process flow.

One problem associated with these approaches is that the resulting recombinant proteins are typically contaminated. This is, for example, a problem for the preparation of nucleic acid-modifying enzymes as the enzyme preparations are typically contaminated with nucleic acids (e.g., RNA and DNA). This contaminating nucleic acid may come not only from the organisms which are the source of the enzyme, but also from unknown organisms present in the reagents and materials used to purify the enzyme after its release from the cells. This is particularly a problem for reverse transcriptase or DNA polymerase enzymes as these are routinely used in techniques of amplification and synthesis of nucleic acid molecules (e.g., the Polymerase Chain Reaction (PCR), particularly RT-PCR). In such example, the presence of contaminating DNA or RNA in the enzyme preparations is a significant problem since it can give rise to spurious amplification or synthesis results. Moreover, growing demand for enzymes as biocatalysts in pharmaceutical applications adds additional level of requirements to the quality of enzyme preparations, such as specificity, lot-to-lot consistency, absence of animal origin contaminants and antibiotics, necessary meet regulatory requirements.

Thus, there remains a need for methods of manufacturing recombinant proteins involving E. coli that are substantially free of contaminants such as nucleic acids. These methods can eliminate the risk of contaminations, for example, environmental and personnel, and enable faster and parallel manufacturing processing in one operation facility. There also remains a need for methods of preparing proteins that are more efficient, cost-effective (for example at pilot and industrial scales), as well as being substantially free of contamination by undesirable cellular components such as nucleic acids.

### BRIEF SUMMARY OF THE DISCLOSURE

In accordance with the present inventions there is provided a method for producing a clarified cell culture comprising a recombinant protein, wherein the method comprises:
i) inoculating a bioreactor with a host cell to provide a cell culture, wherein said host cell is an Escherichia coli transformed with an expression system comprising:
   a) a nucleic acid encoding a recombinant protein operably linked to a first inducible promotor; and
   b) a nucleic acid encoding a T4 lysozyme protein in frame with a nucleic acid encoding PelB secretory signal peptide operably linked to a second inducible promotor,
ii) fermenting the cell culture under conditions that:
a) allow the expression of the recombinant protein; and
   b) allow permeabilization of the cell so as to form a spheroplast that secretes the recombinant protein;
iii) clarifying the fermented cell culture comprising the secreted recombinant protein, wherein said clarifying comprises a primary clarification step using a first clarification filter having a pore size that provides a retention range of at least about 0.4 μm or more, wherein the clarification step does not comprise centrifugation.

Suitably, the method further comprises a secondary clarification step using a second filter having a pore size that provides a retention range of at least about 0.4 μm or more.

Suitably, the primary clarification step and/or secondary clarification step is microfiltration. Preferably, the microfiltration is depth filtration.

Suitably, the microfiltration is selected from normal flow filtration (NFF) or tangential flow filtration (TFF).

Suitably, the primary clarification step uses a first filter having a pore size that provides a retention range of at least about 4.0 to about 9.0 μm, at least about 1.0 to about 3.0 μm, or at least about 0.4 to about 0.8 μm.

Suitably, the secondary clarification step uses a second filter having a pore size that provides a retention range of at least about 1.0 to about 3.0 μm or at least about 0.4 to about 0.8 μm.

Suitably, the primary clarification step and the secondary clarification step use a filter having the same pore size.

Suitably, the clarified cell culture has a turbidity of about less than 20 NTU.

Suitably, the method further comprises a flocculation step prior to the primary clarification step. Preferably, said flocculation step comprises addition of a flocculation agent to the cell culture. Preferably, the flocculation agent is polyethyleneimine (PEI).

Suitably, the method further comprises a nucleic acid inactivation step prior to the primary clarification step. Preferably, said nucleic acid inactivation step comprises addition of a nucleic acid inactivation agent to the cell culture. Preferably, said nucleic acid inactivation agent is benzonase.

Suitably, said fermentation step comprises addition of a stabilizing agent to the cell culture.

Suitably, the method is performed in a closed system, such that a sterile flow path is maintained between the bioreactor and the first each of the first and second clarification filters.

Suitably, the method further comprises a step of subjecting said clarified cell culture to one or more steps of purification of said recombinant protein.

In a further aspect the invention provides a recombinant protein produced by a method of as described herein.

The present invention also relates to the production of vitronectin (VTN).

Accordingly, in accordance with the present inventions there is provided a method for producing a clarified cell culture comprising a recombinant protein, wherein the method comprises:
i) inoculating a bioreactor with a host cell to provide a cell culture, wherein said host cell is an Escherichia coli transformed with an expression system comprising:
   a) a nucleic acid encoding a recombinant protein operably linked to a first inducible promotor, wherein the protein is victronectin (VTN); and
   b) a nucleic acid encoding a T4 lysozyme protein in frame with a nucleic acid encoding PelB secretory signal peptide operably linked to a second inducible promotor,
ii) fermenting the cell culture under conditions that:
   a) allow the expression of the recombinant protein; and
   b) allow permeabilization of the cell so as to form a spheroplast that secretes the recombinant protein;
iii) clarifying the fermented cell culture comprising the secreted recombinant protein, wherein said clarifying comprises
a) a nucleic acid inactivation step prior to the primary clarification step comprising addition of a nucleic acid inactivation agent to the cell culture, optionally wherein the nucleic acid inactivation agent is benzonase; and
b) a primary clarification step using a first clarification filter having a pore size that provides a retention range of at least about 0.4 μm or more, wherein the clarification step does not comprise centrifugation.

Suitably, in the methods of the invention, the method further comprises a secondary clarification step using a second filter having a pore size that provides a retention range of at least about 0.4 μm or more.

Suitably, in the methods of the invention, the primary clarification step uses a first filter having a pore size that provides a retention range of at least about 1.0 to about 3.0 μm.

Suitably, in the methods of the invention, the secondary clarification step uses a second filter having a pore size that provides a retention range of at least about 0.22 μm, at least about 1.0 to about 3.0 μm or at least about 0.4 to about 0.8 μm.

Suitably, in the methods of the invention, the primary clarification step and/or secondary clarification step is microfiltration.

Suitably, in the methods of the invention, microfiltration is depth filtration.

Suitably, in the methods of the invention, the microfiltration is selected from normal flow filtration (NFF) or tangential flow filtration (TFF).

Suitably, in the methods of the invention, the clarified cell culture has a turbidity of about less than 20 NTU.

Suitably, in the methods of the invention, said fermentation step comprises addition of a stabilizing agent to the cell culture.

Suitably, in the methods of the invention, the method is performed in a closed system, such that a sterile flow path is maintained between the bioreactor and the first each of the first and second clarification filters.

Suitably, in the methods of the invention, the method further comprises a step of subjecting said clarified cell culture to one or more steps of purification of said recombinant protein.

The invention also provides a method of producing a protein comprising:
i) culturing a recombinant Escherichia coli cell under conditions that:
   a) allow the expression of the protein; and
   b) allow the permeabilization the cell so as to form a spheroplast that secretes the protein; and
(ii) isolating the protein from the culture without complete lysis of the recombinant cell, wherein the cell comprises an expression system comprising:
   a) a nucleic acid encoding a protein operably linked to a first inducible promoter, wherein the protein is vitronectin (VTN); and
   b) a nucleic acid encoding a T4 lysozyme in frame with a nucleic acid encoding PelB secretory signal peptide operably linked to a second inducible promoter.

Suitably, the methods of the invention does not include: (i) mechanical disruption of the cells; (ii) addition of an exogenous enzyme that degrades the cell wall; and (iii) transformation of the cell with an expression vectors containing the nucleic acid encoding an exogenous enzyme that degrades the cytoplasmic membrane.

Suitably, in the methods of the invention, the nucleic acid encoding a protein operably linked to a first inducible promoter is located on a first construct and the a nucleic acid encoding a T4 lysozyme in frame with a nucleic acid encoding PelB secretory signal peptide is located on a second construct.

Suitably, in the methods of the invention, the first inducible promoter and the second inducible promoter are sequentially induced.

Suitably, in the methods of the invention, the first inducible promoter and the second inducible promoter are simultaneously induced.

Suitably, in the methods of the invention, the first inducible promoter is an IPTG inducible promoter, optionally wherein the IPTG inducible promoter is PT7.

Suitably, in the methods of the invention, the second inducible promoter is an arabinose inducible promoter, optionally wherein the arabinose inducible promoter is araB.

Suitably, in the methods of the invention, the T4 lysozyme enzyme is encoded by gene E.

Suitably, in the methods of the invention, the E. coli is Escherichia coli B strain.

Suitably, in the methods of the invention, fermenting or culturing comprises a change in temperature first temperature to a second temperature, optionally wherein the second temperature is lower than the first temperature.

The invention further provides a recombinant protein produced by the methods of the invention.

The present invention also relates to the production of Inorganic Pyrophosphatase (IPP).

Accordingly, in accordance with the present inventions there is provided a method for producing a clarified cell culture comprising a recombinant protein, wherein the method comprises:
i) inoculating a bioreactor with a host cell to provide a cell culture, wherein said host cell is an Escherichia coli transformed with an expression system comprising:
   a) a nucleic acid encoding a recombinant protein operably linked to a first inducible promotor, wherein the protein is Inorganic Pyrophosphatase (IPP); and
   b) a nucleic acid encoding a T4 lysozyme protein in frame with a nucleic acid encoding PelB secretory signal peptide operably linked to a second inducible promotor,
ii) fermenting the cell culture under conditions that:
   a) allow the expression of the recombinant protein; and
   b) allow permeabilization of the cell so as to form a spheroplast that secretes the recombinant protein;
iii) clarifying the fermented cell culture comprising the secreted recombinant protein, wherein said clarifying comprises a primary clarification step using a first clarification filter having a pore size that provides a retention range of at least about 0.4 μm or more, wherein the clarification step does not comprise centrifugation.

Suitably, in the methods of the invention, the method further comprises a secondary clarification step using a second filter having a pore size that provides a retention range of at least about 0.4 μm or more.

Suitably, in the methods of the invention, the primary clarification step uses a first filter having a pore size that provides a retention range of at least about 4.0 to about 9.0 μm.

Suitably, in the methods of the invention, the secondary clarification step uses a second filter having a pore size that provides a retention range of at least about 0.4 to about 0.8 μm.

Suitably, in the methods of the invention, the primary clarification step and/or secondary clarification step is microfiltration.

Suitably, in the methods of the invention, the microfiltration is depth filtration.

Suitably, in the methods of the invention, the microfiltration is selected from normal flow filtration (NFF) or tangential flow filtration (TFF).

Suitably, in the methods of the invention, the clarified cell culture has a turbidity of about less than 20 NTU.

Suitably, in the methods of the invention, said fermentation step comprises addition of a stabilizing agent to the cell culture.

Suitably, in the methods of the invention, the method is performed in a closed system, such that a sterile flow path is maintained between the bioreactor and the first each of the first and second clarification filters.

Suitably, in the methods of the invention, further comprise a step of subjecting said clarified cell culture to one or more steps of purification of said recombinant protein.

The invention also provides a method of producing a protein comprising:
i) culturing a recombinant Escherichia coli cell under conditions that:
a) allow the expression of the protein; and
b) allow the permeabilization the cell so as to form a spheroplast that secretes the protein; and
(ii) isolating the protein from the culture without complete lysis of the recombinant cell, wherein the cell comprises an expression system comprising:
a) a nucleic acid encoding a protein operably linked to a first inducible promoter, wherein the protein is Inorganic Pyrophosphatase (IPP); and
b) a nucleic acid encoding a T4 lysozyme in frame with a nucleic acid encoding PelB secretory signal peptide operably linked to a second inducible promoter.

Suitably, the methods of the invention does not include: (i) mechanical disruption of the cells; (ii) addition of an exogenous enzyme that degrades the cell wall; and (iii) transformation of the cell with an expression vectors containing the nucleic acid encoding an exogenous enzyme that degrades the cytoplasmic membrane.

Suitably, in the methods of the invention, the nucleic acid encoding a protein operably linked to a first inducible promoter is located on a first construct and the a nucleic acid encoding a T4 lysozyme in frame with a nucleic acid encoding PelB secretory signal peptide is located on a second construct.

Suitably, in the methods of the invention, the first inducible promoter and the second inducible promoter are sequentially induced.

Suitably, in the methods of the invention, the first inducible promoter and the second inducible promoter are simultaneously induced.

Suitably, in the methods of the invention, the first inducible promoter is an IPTG inducible promoter, optionally wherein the IPTG inducible promoter is PT7.

Suitably, in the methods of the invention, the second inducible promoter is an arabinose inducible promoter, optionally wherein the arabinose inducible promoter is araB.

Suitably, in the methods of the invention, the T4 lysozyme enzyme is encoded by gene E.

Suitably, in the methods of the invention, the E. coli is Escherichia coli B strain.

Suitably, in the methods of the invention, fermenting or culturing comprises a change in temperature first temperature to a second temperature, optionally wherein the second temperature is lower than the first temperature.

The invention further provides a recombinant protein produced by the methods of the invention.

The present invention also relates to the production of Moloney Murine Leukemia Virus Reverse Transcriptase (MMLV-RT).

Accordingly, In accordance with the present inventions there is provided a method for producing a clarified cell culture comprising a recombinant protein, wherein the method comprises:
i) inoculating a bioreactor with a host cell to provide a cell culture, wherein said host cell is an Escherichia coli transformed with an expression system comprising:
a) a nucleic acid encoding a recombinant protein operably linked to a first inducible promotor, wherein the protein is Moloney Murine Leukemia Virus Reverse Transcriptase (MMLV-RT); and
b) a nucleic acid encoding a T4 lysozyme protein in frame with a nucleic acid encoding PelB secretory signal peptide operably linked to a second inducible promotor, ii) fermenting the cell culture under conditions that:
a) allow the expression of the recombinant protein; and
b) allow permeabilization of the cell so as to form a spheroplast that secretes the recombinant protein,
wherein said step of fermenting comprises addition of a stabilizing agent to the cell culture;
iii) clarifying the fermented cell culture comprising the secreted recombinant protein, wherein said clarifying comprises:
a) addition of a flocculation agent to the cell culture, wherein the flocculation agent is polyethyleneimine (PEI);
b) a primary clarification of the flocculated cell culture using a first clarification filter having a pore size that provides a retention range of at least about 0.4 μm or more, wherein the clarification step does not comprise centrifugation.

Suitably, in the methods of the invention, the method further comprises a secondary clarification step using a second filter having a pore size that provides a retention range of at least about 0.22 μm.

Suitably, in the methods of the invention, the primary clarification step uses a first filter having a pore size that provides a retention range of at least about 0.4 to about 0.8 μm.

Suitably, in the methods of the invention, the secondary clarification step uses a second filter having a pore size that provides a retention range of at least about 0.4 to about 0.8 μm or at least about 1.0 to about 3.0 μm.

Suitably, in the methods of the invention, the primary clarification step and/or secondary clarification step is microfiltration, optionally where in the microfiltration is depth filtration.

Suitably, in the methods of the invention, the microfiltration is selected from normal flow filtration (NFF) or tangential flow filtration (TFF).

Suitably, in the methods of the invention, the clarified cell culture has a turbidity of about less than 20 NTU.

Suitably, in the methods of the invention, the method is performed in a closed system, such that a sterile flow path is maintained between the bioreactor and the first each of the first and second clarification filters.

Suitably, in the methods of the invention, the method further comprising a step of subjecting said clarified cell culture to one or more steps of purification of said recombinant protein.

Suitably, in the methods of the invention, the stabilizing agent comprises Sucrose, Na₂SO₄ and Brij 35, optionally 1M Sucrose, 0.5M Na₂SO₄ and 0.5% Brij 35.

The invention also provides a method of producing a protein comprising:
i) culturing a recombinant Escherichia coli cell under conditions that:
a) allow the expression of the protein; and
b) allow the permeabilization the cell so as to form a spheroplast that secretes the protein; and
(ii) isolating the protein from the culture without complete lysis of the recombinant cell, wherein the cell comprises an expression system comprising:
a) a nucleic acid encoding a protein operably linked to a first inducible promoter, wherein the protein is Moloney Murine Leukemia Virus Reverse Transcriptase (MMLV-RT); and
b) a nucleic acid encoding a T4 lysozyme in frame with a nucleic acid encoding PelB secretory signal peptide operably linked to a second inducible promoter.

Suitably, the methods of the invention does not include: (i) mechanical disruption of the cells; (ii) addition of an exogenous enzyme that degrades the cell wall; and (iii) transformation of the cell with an expression vectors containing the nucleic acid encoding an exogenous enzyme that degrades the cytoplasmic membrane.

Suitably, in the methods of the invention, the nucleic acid encoding a protein operably linked to a first inducible promoter is located on a first construct and the a nucleic acid encoding a T4 lysozyme in frame with a nucleic acid encoding PelB secretory signal peptide is located on a second construct.

Suitably, in the methods of the invention, the first inducible promoter and the second inducible promoter are sequentially induced.

Suitably, in the methods of the invention, the first inducible promoter and the second inducible promoter are simultaneously induced.

Suitably, in the methods of the invention, the first inducible promoter is an IPTG inducible promoter, optionally wherein the IPTG inducible promoter is Ptac or PT7.

Suitably, in the methods of the invention, the second inducible promoter is an arabinose inducible promoter, optionally wherein the arabinose inducible promoter is araB.

Suitably, in the methods of the invention, the T4 lysozyme enzyme is encoded by gene E.

Suitably, in the methods of the invention, the E. coli is Escherichia coli strain JS007 or Escherichia coli B strain.

Suitably, in the methods of the invention, fermenting or culturing comprises a change in temperature first temperature to a second temperature, optionally wherein the second temperature is lower than the first temperature.

The invention further provides a recombinant protein produced by the methods of the invention.

The present invention also relates to the production ribonuclease inhibitor (RI).

Accordingly, in accordance with the present inventions there is provided a method for producing a clarified cell culture comprising a recombinant protein, wherein the method comprises:
i) inoculating a bioreactor with a host cell to provide a cell culture, wherein said host cell is an Escherichia coli transformed with an expression system comprising:
a) a nucleic acid encoding a recombinant protein operably linked to a first inducible promotor, wherein the protein is ribonuclease inhibitor (RI); and
b) a nucleic acid encoding a T4 lysozyme protein in frame with a nucleic acid encoding PelB secretory signal peptide operably linked to a second inducible promotor,
ii) fermenting the cell culture under conditions that:
a) allow the expression of the recombinant protein; and
b) allow permeabilization of the cell so as to form a spheroplast that secretes the recombinant protein,
wherein said step of fermenting comprises addition of a stabilizing agent to the cell culture;
iii) clarifying the fermented cell culture comprising the secreted recombinant protein, wherein said clarifying comprises:
a) addition of a flocculation agent to the cell culture, wherein the flocculation agent is polyethyleneimine (PEI);
b) a primary clarification of the flocculated cell culture using a first clarification filter having a pore size that provides a retention range of at least about 0.4 μm or more, wherein the clarification step does not comprise centrifugation.

Suitably, the methods of the invention, further comprise a secondary clarification step using a second filter having a pore size that provides a retention range of at least about 0.22 μm.

Suitably, in the methods of the invention, the primary clarification step uses a first filter having a pore size that provides a retention range of at least about 0.4 to about 0.8 μm.

Suitably, in the methods of the invention, the secondary clarification step uses a second filter having a pore size that provides a retention range of at least about 0.4 to about 0.8 μm or at least about 1.0 to about 3.0 μm.

Suitably, in the methods of the invention, the primary clarification step and/or secondary clarification step is microfiltration, optionally where in the microfiltration is depth filtration.

Suitably, in the methods of the invention, the microfiltration is selected from normal flow filtration (NFF) or tangential flow filtration (TFF).

Suitably, in the methods of the invention, the clarified cell culture has a turbidity of about less than 20 NTU.

Suitably, in the methods of the invention, the method is performed in a closed system, such that a sterile flow path is maintained between the bioreactor and the first each of the first and second clarification filters.

Suitably, the methods of the invention, further comprise a step of subjecting said clarified cell culture to one or more steps of purification of said recombinant protein.

Suitably, in the methods of the invention, the stabilizing agent comprises Sucrose, Na₂SO₄ and Brij 35, optionally 1M Sucrose, 0.5M Na₂SO₄ and 0.5% Brij 35.

The invention also provides a method of producing a protein comprising:
i) culturing a recombinant Escherichia coli cell under conditions that:
a) allow the expression of the protein; and
b) allow the permeabilization the cell so as to form a spheroplast that secretes the protein; and
(ii) isolating the protein from the culture without complete lysis of the recombinant cell, wherein the cell comprises an expression system comprising:
a) a nucleic acid encoding a protein operably linked to a first inducible promoter, wherein the protein is ribonuclease inhibitor (RI); and
b) a nucleic acid encoding a T4 lysozyme in frame with a nucleic acid encoding PelB secretory signal peptide operably linked to a second inducible promoter.

Suitably, the methods of the invention does not include: (i) mechanical disruption of the cells; (ii) addition of an exogenous enzyme that degrades the cell wall; and (iii) transformation of the cell with an expression vectors containing the nucleic acid encoding an exogenous enzyme that degrades the cytoplasmic membrane.

Suitably, in the methods of the invention, the nucleic acid encoding a protein operably linked to a first inducible promoter is located on a first construct and the a nucleic acid encoding a T4 lysozyme in frame with a nucleic acid encoding PelB secretory signal peptide is located on a second construct.

Suitably, in the methods of the invention, the first inducible promoter and the second inducible promoter are sequentially induced.

Suitably, in the methods of the invention, the first inducible promoter and the second inducible promoter are simultaneously induced.

Suitably, in the methods of the invention, the first inducible promoter is an IPTG inducible promoter, optionally wherein the IPTG inducible promoter is PT7.

Suitably, in the methods of the invention, the second inducible promoter is an arabinose inducible promoter, optionally wherein the arabinose inducible promoter is araB.

Suitably, in the methods of the invention, the T4 lysozyme enzyme is encoded by gene E.

Suitably, in the methods of the invention, the E. coli is Escherichia Coli strain JS007 or Escherichia coli B strain.

Suitably, in the methods of the invention, fermenting or culturing comprises a change in temperature first temperature to a second temperature, optionally wherein the second temperature is lower than the first temperature.

The invention further provides a recombinant protein produced by the methods of the invention.

The present invention also relates to the production of 2-O-methyltransferase (OMT).

Accordingly, In accordance with the present inventions there is provided a method for producing a clarified cell culture comprising a recombinant protein, wherein the method comprises:
i) inoculating a bioreactor with a host cell to provide a cell culture, wherein said host cell is an Escherichia coli transformed with an expression system comprising:
a) a nucleic acid encoding a recombinant protein operably linked to a first inducible promotor, wherein the protein is 2-O-methyltransferase (OMT); and
b) a nucleic acid encoding a T4 lysozyme protein in frame with a nucleic acid encoding PelB secretory signal peptide operably linked to a second inducible promotor,
ii) fermenting the cell culture under conditions that:
a) allow the expression of the recombinant protein; and
b) allow permeabilization of the cell so as to form a spheroplast that secretes the recombinant protein,
wherein said step of fermenting comprises addition of a stabilizing agent to the cell culture;
iii) clarifying the fermented cell culture comprising the secreted recombinant protein, wherein said clarifying comprises:
a) addition of a flocculation agent to the cell culture, wherein the flocculation agent is polyethyleneimine (PEI);
b) a primary clarification of the flocculated cell culture using a first clarification filter having a pore size that provides a retention range of at least about 0.4 μm or more, wherein the clarification step does not comprise centrifugation.

Suitably, in the methods of the invention, the method further comprises a secondary clarification step using a second filter having a pore size that provides a retention range of at least about 0.22 μm.

Suitably, in the methods of the invention, the primary clarification step uses a first filter having a pore size that provides a retention range of at least about 0.4 to about 0.8 μm.

Suitably, in the methods of the invention, the secondary clarification step using a second filter having a pore size that provides a retention range of at least abut 0.4-0.8 or at least about 1.0 to about 3.0 μm.

Suitably, in the methods of the invention, the primary clarification step and/or secondary clarification step is microfiltration, optionally where in the microfiltration is depth filtration.

Suitably, in the methods of the invention, the microfiltration is selected from normal flow filtration (NFF) or tangential flow filtration (TFF).

Suitably, in the methods of the invention, the clarified cell culture has a turbidity of about less than 20 NTU.

Suitably, in the methods of the invention, the method is performed in a closed system, such that a sterile flow path is maintained between the bioreactor and the first each of the first and second clarification filters.

Suitably, in the methods of the invention, further comprises a step of subjecting said clarified cell culture to one or more steps of purification of said recombinant protein.

Suitably, in the methods of the invention, the stabilizing agent comprises Sucrose, Na₂SO₄ and Brij 35, optionally 1M Sucrose, 0.5M Na₂SO₄ and 0.5% Brij 35.

The invention also provides a method of producing a protein comprising:
i) culturing a recombinant Escherichia coli cell under conditions that:
a) allow the expression of the protein; and
b) allow the permeabilization the cell so as to form a spheroplast that secretes the protein; and
(ii) isolating the protein from the culture without complete lysis of the recombinant cell, wherein the cell comprises an expression system comprising:
a) a nucleic acid encoding a protein operably linked to a first inducible promoter, wherein the protein is 2-O-methyltransferase (OMT); and
b) a nucleic acid encoding a T4 lysozyme in frame with a nucleic acid encoding PelB secretory signal peptide operably linked to a second inducible promoter.

Suitably, the methods of the invention does not include: (i) mechanical disruption of the cells; (ii) addition of an exogenous enzyme that degrades the cell wall; and (iii) transformation of the cell with an expression vectors containing the nucleic acid encoding an exogenous enzyme that degrades the cytoplasmic membrane.

Suitably, in the methods of the invention, the nucleic acid encoding a protein operably linked to a first inducible promoter is located on a first construct and the a nucleic acid encoding a T4 lysozyme in frame with a nucleic acid encoding PelB secretory signal peptide is located on a second construct.

Suitably, in the methods of the invention, the first inducible promoter and the second inducible promoter are sequentially induced.

Suitably, in the methods of the invention, the first inducible promoter and the second inducible promoter are simultaneously induced.

Suitably, in the methods of the invention, the first inducible promoter is an IPTG inducible promoter, optionally wherein the IPTG inducible promoter is PT7.

Suitably, in the methods of the invention, the second inducible promoter is an arabinose inducible promoter, optionally wherein the arabinose inducible promoter is araB.

Suitably, in the methods of the invention, the T4 lysozyme enzyme is encoded by gene E.

Suitably, in the methods of the invention, the E. coli is Escherichia Coli strain JS007 or Escherichia coli B strain.

Suitably, in the methods of the invention, fermenting or culturing comprises a change in temperature first temperature to a second temperature, optionally wherein the second temperature is lower than the first temperature.

The invention further provides a recombinant protein produced by the methods of the invention.

### BRIEF DESCRIPTION OF THE DRAWINGS

Embodiments of the invention are further described hereinafter with reference to the accompanying drawings, in which:

FIG. 1 shows the structures of plasmids: (A) pET29-optVTN, (B) T4 gene gpe expression vector pACYC184-PT7-pelB-gpe-lacl

FIG. 2 shows a microscopical view of E. coli cells before and after induction of gpe gene expression.

FIG. 3 shows the transformation of two different E. coli strains with target plasmids. (A) culture growth when JS007 E. coli strain and BL21 (DE3) E. coli strain were transformed with the target plasmids. Point of expression induction with 0.1 mM IPTG is noted in the graph (OD600 1.0-1.5). After 7 h of incubation, optical density of the BL21 (DE3) culture was 12.40, whereas optical density of the JS007 culture was 6.60. (B) VTN protein expression results in JS007 strain (on the left) and BL21 (DE3) strain (on the right). Legend: (1), (6)—Page Ruler Prestained Protein Ladder (Thermo Scientific); (2), (7)—total protein fraction before the induction; (3), (8)—total protein fraction after the induction (0.1 mM IPTG, 4.5 h at 25° C.); (4), (9)—soluble protein fraction after induction; (5), (10)—insoluble protein fraction after induction. Target proteins are indicated with the arrows.

FIG. 4 shows a comparison of scale up fermentations in flask, Biostat A, Biostat C and SUF. (A) comparison of biomass and media fractions of different scale fermentations. Legend: (5), (10),—Page Ruler Prestained Protein Ladder (Thermo Scientific); (1)—biomass, biostat A before induction; (2)—biomass, flask after induction; (3)—biomass, biostat A after induction; (4)—biomass SUF, after induction; (6)—medium, biostat A before induction; (7)—medium, flask after induction; (8)—medium, biostat A after induction; (9)—medium, SUF after induction; SDS PAGE gel 4-12% Bolt. (B) Typical fermentation process profile in Biostat A.

FIG. 5 shows reproducibility of VTN fermentation process in large scale 300 L SUF. (A) fermentation profiles of three fermentations. (B) SDS PAGE analysis of supernatant fractions after fermentation process of separate fermentations. Legend: (1) Page Ruler Prestained Protein Ladder (Thermo Scientific); (2), (3), (4)—supernatant fraction after autolysis of different fermentations in 300 L SUF. 4-12% Bolt SDS gel.

FIG. 6 shows structures of plasmids: (A) target gene expression vector pET21b-ppa-Kn, (B) T4 gene gpe expression vector pAra-pelB-gpe.

FIG. 7 shows a microscopical view of E. coli cells before and after induction of gpe gene expression.

FIG. 8 shows a 12% SDS-PAGE of E. coli BL21(DE3) [pAra-pelB-gpe]/pET21b-ppa-Kn] IPP expression with lysis comparison in 5 L (Biostat A) and flask scales: (1) -PageRuler™ Prestained Protein Ladder (Thermo Fisher), (2)—Biomass sample before induction in Biostat A (70 pg, normalized by OD), (3)—Biomass sample after induction in Biostat A (70 pg, normalized by OD), (4)—Biomass sample before induction in flask (70 pg, normalized by OD), (5)—Biomass sample after induction in flask (70 pg, normalized by OD), (6)—PageRuler™ Prestained Protein Ladder (Thermo Fisher), (7)—Supernatant sample before induction in Biostat A (5.25 pl), (8)—Supernatant sample after induction in Biostat A (5.25 pl), (9)—Supernatant sample before induction in flask (5.25 pl), (10) -Supernatant sample after induction in flask (5.25 pl), (11)—PageRuler™ Prestained Protein Ladder (Thermo Fisher).

FIG. 9 shows a comparison of IPP expression level in E. coli BL21(DE3) [pAra-pelB-gpe]/[pET21b-ppa-Kn]media using basal T4 lysozyme production feature in 5 L Biostat A and flask after 3 and 20 hours: (1) PageRuler™ Prestained Protein Ladder (Thermo Fisher), (2) media sample before the induction of IPP in flask (5.25 pl), (3) media sample 3 hours post-induction of IPP in flask (5.25 μl), (4)—media sample 20 hours post-induction of IPP in flask (5.25 μl), (5) media sample before the induction of IPP in Biostat A (5.25 μl), (6) media sample 3 hours post-induction of IPP Biostat A (5.25 μl), (7) media sample 20 hours post-induction of IPP Biostat A (5.25 μl).

FIG. 10 shows the IPP expression process in BiostatA stirred bioreactor.

FIG. 11 shows the IPP expression process in 300 L SUF bioreactor.

FIG. 12 shows structures of plasmids: (A) target gene expression vector pMuLV-RT-Km, (B) T4 gene gpe expression vector pAra-pelB-gpe.

FIG. 13 shows a microscopical view of E. coli cells before and after induction of gpe gene expression.

FIG. 14 shows the optical density growth curve of E. coli JS007 pMuLV-RT-Km, pAra-pelB-gpe culture.

FIG. 15 shows a 12% SDS-PAGE of E. coli JS007 pMuLV-RT-Km, pAra-pelB-gpe culture biomass and medium samples. (1)—PageRuler™ Prestained Protein Ladder (Thermo Fisher), (2)—culture medium sample before induction of target; (3)—culture medium sample after 5 hours of target gene induction 0.1 mM IPTG, (4)—culture medium sample after 2 hours induction of lysis gene by 0.5 g/l L-arabinose and 7 hours induction of target gene 0.1 mM IPTG, (5)—PageRuler™ Prestained Protein Ladder Plus (Thermo Fisher), (6)—biomass sample before induction of target; (7)—biomass sample after 5 hours of target gene induction 0.1 mM IPTG, (8)—biomass sample after 2 hours induction of lysis gene by 0.5 g/l L-arabinose and 7 hours induction of target gene 0.1 mM IPTG.

FIG. 16 shows MuLV-RT expression in Biostat A.

FIG. 17 shows shows structures of plasmids: (A) target gene expression vector pET29-optRNAseOUT, (B) T4 gene gpe expression vector pAra-pelB-gpe.

FIG. 18 shows a microscopical view of E. coli cells before and after induction of gpe gene expression.

FIG. 19 shows an SDS PAGE analysis of Ribonuclease Inhibitor protein induction. (1) PageRuler™ Prestained Protein Ladder, 10 to 250 kDa (Thermo Fisher); (2) culture medium; (3) E. coli JS008 pET29-optRNAseOUT, pAra-pelB-gpe culture media without target gene induction; (4) E. coli JS008 pET29-optRNAseOUT, pAra-pelB-gpe culture media after target gene induction.

FIG. 20 shows ribonuclease inhibitor (RI) expression in Biostat A.

FIG. 21 shows RI culture growth dynamics comparison in Biostat A bioreactor systems (inoculums from RCB and from fresh transformation).

FIG. 22 shows an SDS PAGE analysis of Ribonuclease Inhibitor protein and lysozyme induction. (1) PageRuler™ Prestained Protein Ladder, 10 to 250 kDa (Thermo Fisher); (2) E. coli JS008 pET29-optRNAseOUT, pAra-pelB-gpe RCB culture media before target gene induction; (3) E. coli JS008 pET29-optRNAseOUT, pAra-pelB-gpe RCB culture media 1.5 h after target gene induction; (4) E. coli JS008 pET29-optRNAseOUT, pAra-pelB-gpe RCB culture media 3 h after target gene induction; (5) E. coli JS008 pET29-optRNAseOUT, pAra-pelB-gpe RCB supernatant after arabinose induction; (6) 2× diluted E. coli JS008 pET29-optRNAseOUT, pAra-pelB-gpe RCB supernatant after osmotic shock; (7) 4× diluted E. coli JS008 pET29-optRNAseOUT, pAra-pelB-gpe RCB supernatant after osmotic shock; (8) 8× diluted E. coli JS008 pET29-optRNAseOUT, pAra-pelB-gpe RCB supernatant after osmotic shock; (9) 2× diluted E. coli JS008 pET29-optRNAseOUT, pAra-pelB-gpe RCB supernatant after osmotic shock performed in manufacturing unit; (10) 4× diluted E. coli JS008 pET29-optRNAseOUT, pAra-pelB-gpe RCB supernatant after osmotic shock in manufacturing unit; (11) 8× diluted E. coli JS008 pET29-optRNAseOUT, pAra-pelB-gpe RCB supernatant after osmotic shock in manufacturing unit; (12) 2× diluted E. coli JS008 pET29-optRNAseOUT, pAra-pelB-gpe RCB supernatant after osmotic shock (after disintegration with ultrasound); (13) PageRuler™ Prestained Protein Ladder, 10 to 250 kDa (Thermo Fisher); (14) E. coli JS008 pET29-optRNAseOUT, pAra-pelB-gpe fresh transformant culture media before target gene induction; (15) E. coli JS008 pET29-optRNAseOUT, pAra-pelB-gpe fresh transformant culture media 1.5 h after target gene induction; (16) E. coli JS008 pET29-optRNAseOUT, pAra-pelB-gpe fresh transformant culture media 3 h after target gene induction; (17) E. coli JS008 pET29-optRNAseOUT, pAra-pelB-gpe fresh transformant supernatant after arabinose induction; (18) 2× diluted E. coli JS008 pET29-optRNAseOUT, pAra-pelB-gpe fresh transformant supernatant after osmotic shock; (19) 4× diluted E. coli JS008 pET29-optRNAseOUT, pAra-pelB-gpe fresh transformant supernatant after osmotic shock; (20) 8× diluted E. coli JS008 pET29-optRNAseOUT, pAra-pelB-gpe fresh transformant supernatant after osmotic shock; (21) 2× diluted E. coli JS008 pET29-optRNAseOUT, pAra-pelB-gpe fresh transformant supernatant after osmotic shock performed in manufacturing unit; (22) 4× diluted E. coli JS008 pET29-optRNAseOUT, pAra-pelB-gpe fresh transformant supernatant after osmotic shock in manufacturing unit; (23) 8× diluted E. coli JS008 pET29-optRNAseOUT, pAra-pelB-gpe fresh transformant supernatant after osmotic shock in manufacturing unit; (24) 2× diluted E. coli JS008 pET29-optRNAseOUT, pAra-pelB-gpe fresh transformant supernatant after osmotic shock (after disintegration with ultrasound).

FIG. 23 shows structures of plasmids: (A) target gene expression vector pLATE31-VP39delta32-KnR, (B) T4 gene gpe expression vector pACYC184-PT7-pelB-gpe-lacl

FIG. 24 shows a microscopical view of E. coli cells before and after induction of gpe gene expression.

FIG. 25 shows an SDS PAGE gel of JS007/pLATE31-VP39delta32-KnR/pACYC-PT7-pelB-gpe-lacl culture cells extract and media samples after 2-O-methyltransferase induction. (1, 7 and 13) PageRuler™ Plus Prestained Protein Ladder, 10 to 250 kDa (Thermo Fisher). (2) cells sample after 1 hour protein synthesis induction; (3) cells sample after 2 hours protein synthesis induction; (4) cells sample after 3 hours protein synthesis induction; (5) cells sample after 5 hours protein synthesis induction; (6) cells sample after 17 hours protein synthesis induction; (8) media sample after 1 hour protein synthesis induction; (9) media sample after 2 hours protein synthesis induction; (10) media sample after 3 hours protein synthesis induction; (11) media sample after 5 hours protein synthesis induction; (12) media sample after 17 hours protein synthesis induction; the 2-O-methyltransferase released in media is indicated by arrow.

FIG. 26 shows OMT expression in Biostat.

FIG. 27 shows OMT culture growth dynamics comparison in Biostat A bioreactor systems (4 h and overnight (OV) feeding time).

FIG. 28 shows OMT activity dynamics comparison in Biostat A bioreactor systems with and without autolysis (4 h and overnight (OV) feeding time).

FIG. 29 shows structures of plasmids: (A) cas9 recombinant plasmid scheme—target gene expression vector pET21-Cas9V2; (B) T4 gene gpe expression vector pACYC184-PT7-pelB-gpe-lacl.

FIG. 30 shows a microscopical view of E. coli cells before and after induction of gpe gene expression.

FIG. 31 shows an SDS PAGE gel analysis of optimizing medium, IPTG induction concentration and time for Cas9 protein expression in BL21/pET21-Cas9V2/pACYC-PT7-pelB-gpe-lacl culture.), (2)—total protein fraction before the induction; (3) media sample (supernatant) induced with 0.1 mM IPTG and cultivated for 4 h (4) media sample induced with 0.1 mM IPTG and cultivated for 22 h; (5) media sample induced with 0.5 mM IPTG and cultivated for 4 h (6) sample induced with 0.5 mM IPTG and cultivated for 22 h; (1, 7) Page Ruler Prestained Protein Ladder (Thermo Scientific).

FIG. 32 shows an SDS PAGE gel showing autolysis and Cas-9 protein expression confirmation experiments in separate bioreactors (Biostat A). Cas-9 protein expression was performed in fermenters F2-F5. Cells were induced at different induction points (OD: 2,3; 2,6; 2,9; 2,7). Samples before and after induction were taken and soluble and insoluble fractions separated by centrifugation. Soluble fraction (supernatant) analysis gel (A) (1); (10) Page Ruler Prestained Protein Ladder (2) F2 before induction (3) F3 before induction (4) F4 before induction (5) F5 before induction (6) F2 after induction (7) F3 after induction (8) F4 after induction (9) F5 after induction. Analysis of insoluble fraction (cell debris), gel (B) (1); (10) Page Ruler Prestained Protein Ladder (2) F2 before induction (3) F3 before induction (4) F4 before induction (5) F5 before induction (6) F2 after induction (7) F3 after induction (8) F4 after induction (9) F5 after induction. Given results does not show bands of target protein in samples (small lanes that corresponds the size of Cas9 protein −160 kDa might be the proteins of E. coli cell because the same lanes are seen in samples before induction). Results of analysis of soluble and insoluble fraction indicates that most of non-target proteins can be removed during sedimentation and clarification process.

FIG. 33 shows a graph of a typical Cas9 fermentation in a Biostat A fermenter. Temperature is reduced from 37° C. to 24° C. immediately after induction. DO is maintained at 30%, then reduced to 3% 30 min. after induction to limit the potential oxidation of the secreted product. pH is maintained at 7.00±0.05 through the fermentation.

FIG. 34 shows nuclease Cas9 cell culture growth dynamics comparison in Biostat A bioreactor systems (inoculums from RCB—research cell bank), single use fermenter 30 L (SUF 30 L), and master cell bank (MCB). Induction points are marked by arrows.

FIG. 35 shows graphs of two independent fermentations in 300 L SUF to confirm robustness of Cas-9 protein expression and cell lysis. Both processes are highly similar based on the culture growth, oxygen consumption rate (reflected by the stirring speed required to maintain the dissolved oxygen setpoint) and the moment of the glycerol depletion (reflected by the rapid decrease in stirring speed and air flow after a dissolved oxygen concentration peak).

FIG. 36 shows a schematic of the clarification process used to obtain various recombinant proteins such as VTN (vitronectin) and Cas9.

FIG. 37 shows a schematic of the clarification process used to obtain various recombinant proteins such as IPP (Inorganic Pyrophosphatase).

FIG. 38 shows a schematic of the clarification process used to obtain various recombinant proteins such as MMLV-RT (reverse transcriptase), RI (ribonuclease Inhibitor), OMT (2-O-methyltransferase), T7 RNA Polymerase, and bFGF.

FIG. 39 shows a schematic of the clarification process used to obtain various recombinant proteins such as AmpliTaq DNA polymerase.

### DETAILED DESCRIPTION

The reader's attention is directed to all papers and documents which are filed concurrently with or previous to this specification in connection with this application and which are open to public inspection with this specification, and the contents of all such papers and documents are incorporated herein by reference.

Accordingly, the invention provides a method for producing a clarified cell culture comprising a recombinant protein, wherein the method comprises:
i) inoculating a bioreactor with a host cell to provide a cell culture, wherein said host cell is an Escherichia coli transformed with an expression system comprising:
   a) a nucleic acid encoding a recombinant protein operably linked to a first inducible promotor; and
   b) a nucleic acid encoding a T4 lysozyme protein in frame with a nucleic acid encoding PelB secretory signal peptide,
ii) fermenting the cell culture under conditions that:
a) allow the expression of the recombinant protein; and
   b) allow the permeabilization the cell so as to form a spheroplast that secretes the recombinant protein;
iii) clarifying the fermented cell culture comprising the secreted recombinant protein, wherein said clarifying comprises a primary clarification step using a first clarification filter having a pore size that provides a retention range of at least about 0.4 μm or more, wherein the clarification step does not comprise centrifugation.

Accordingly, the invention further provides a method for producing a clarified cell culture comprising a recombinant protein, wherein the method comprises:
i) inoculating a bioreactor with a host cell to provide a cell culture, wherein said host cell is an Escherichia coli transformed with an expression system comprising:
a) a nucleic acid encoding a recombinant protein operably linked to a first inducible promotor, wherein the protein is victronectin (VTN); and
b) a nucleic acid encoding a T4 lysozyme protein in frame with a nucleic acid encoding PelB secretory signal peptide operably linked to a second inducible promotor,
ii) fermenting the cell culture under conditions that:
a) allow the expression of the recombinant protein; and
b) allow permeabilization of the cell so as to form a spheroplast that secretes the recombinant protein;
iii) clarifying the fermented cell culture comprising the secreted recombinant protein, wherein said clarifying comprises
a) a nucleic acid inactivation step prior to the primary clarification step comprising addition of a nucleic acid inactivation agent to the cell culture, optionally wherein the nucleic acid inactivation agent is benzonase;
b) and a primary clarification step using a first clarification filter having a pore size that provides a retention range of at least about 0.4 μm or more, wherein the clarification step does not comprise centrifugation. Accordingly, the invention further provides a method for producing a clarified cell culture comprising a recombinant protein, wherein the method comprises:
i) inoculating a bioreactor with a host cell to provide a cell culture, wherein said host cell is an Escherichia coli transformed with an expression system comprising:
a) a nucleic acid encoding a recombinant protein operably linked to a first inducible promotor, wherein the protein is Inorganic Pyrophosphatase (IPP); and
b) a nucleic acid encoding a T4 lysozyme protein in frame with a nucleic acid encoding PelB secretory signal peptide operably linked to a second inducible promotor,
ii) fermenting the cell culture under conditions that:
a) allow the expression of the recombinant protein; and
b) allow permeabilization of the cell so as to form a spheroplast that secretes the recombinant protein;
iii) clarifying the fermented cell culture comprising the secreted recombinant protein, wherein said clarifying comprises a primary clarification step using a first clarification filter having a pore size that provides a retention range of at least about 0.4 μm or more, wherein the clarification step does not comprise centrifugation.

Accordingly, the invention further provides a method for producing a clarified cell culture comprising a recombinant protein, wherein the method comprises:
i) inoculating a bioreactor with a host cell to provide a cell culture, wherein said host cell is an Escherichia coli transformed with an expression system comprising:
a) a nucleic acid encoding a recombinant protein operably linked to a first inducible promotor, wherein the protein is Moloney Murine Leukemia Virus Reverse Transcriptase (MMLV-RT); and
b) a nucleic acid encoding a T4 lysozyme protein in frame with a nucleic acid encoding PelB secretory signal peptide operably linked to a second inducible promotor,
ii) fermenting the cell culture under conditions that:
   a) allow the expression of the recombinant protein; and
   b) allow permeabilization of the cell so as to form a spheroplast that secretes the recombinant protein,
wherein said step of fermenting comprises addition of a stabilizing agent to the cell culture;
iii) clarifying the fermented cell culture comprising the secreted recombinant protein, wherein said clarifying comprises:
   a) addition of a flocculation agent to the cell culture, wherein the flocculation agent is polyethyleneimine (PEI);
   b) a primary clarification of the flocculated cell culture using a first clarification filter having a pore size that provides a retention range of at least about 0.4 μm or more, wherein the clarification step does not comprise centrifugation.

Accordingly, the invention further provides a method for producing a clarified cell culture comprising a recombinant protein, wherein the method comprises:
i) inoculating a bioreactor with a host cell to provide a cell culture, wherein said host cell is an Escherichia coli transformed with an expression system comprising:
   a) a nucleic acid encoding a recombinant protein operably linked to a first inducible promotor, wherein the protein is ribonuclease inhibitor (RI); and
   b) a nucleic acid encoding a T4 lysozyme protein in frame with a nucleic acid encoding PelB secretory signal peptide operably linked to a second inducible promotor,
ii) fermenting the cell culture under conditions that:
   a) allow the expression of the recombinant protein; and
   b) allow permeabilization of the cell so as to form a spheroplast that secretes the recombinant protein,
wherein said step of fermenting comprises addition of a stabilizing agent to the cell culture;
iii) clarifying the fermented cell culture comprising the secreted recombinant protein, wherein said clarifying comprises:
   a) addition of a flocculation agent to the cell culture, wherein the flocculation agent is polyethyleneimine (PEI);
   b) a primary clarification of the flocculated cell culture using a first clarification filter having a pore size that provides a retention range of at least about 0.4 μm or more, wherein the clarification step does not comprise centrifugation.

Accordingly, the invention further provides a method for producing a clarified cell culture comprising a recombinant protein, wherein the method comprises:
i) inoculating a bioreactor with a host cell to provide a cell culture, wherein said host cell is an Escherichia coli transformed with an expression system comprising:
   a) a nucleic acid encoding a recombinant protein operably linked to a first inducible promotor, wherein the protein is 2-O-methyltransferase (OMT); and
   b) a nucleic acid encoding a T4 lysozyme protein in frame with a nucleic acid encoding PelB secretory signal peptide operably linked to a second inducible promotor,
ii) fermenting the cell culture under conditions that:
   a) allow the expression of the recombinant protein; and
   b) allow permeabilization of the cell so as to form a spheroplast that secretes the recombinant protein,
wherein said step of fermenting comprises addition of a stabilizing agent to the cell culture;
iii) clarifying the fermented cell culture comprising the secreted recombinant protein, wherein said clarifying comprises:
   c) addition of a flocculation agent to the cell culture, wherein the flocculation agent is polyethyleneimine (PEI);
   d) a primary clarification of the flocculated cell culture using a first clarification filter having a pore size that provides a retention range of at least about 0.4 μm or more, wherein the clarification step does not comprise centrifugation.

Advantageously, the inventors have found that recombinant E. coli spheroplasts secrete recombinant protein prior to cell lysis. The inventors have demonstrated that coordinated expression of nucleic acid encoding a T4 lysozyme linked to a pectate lyase B (PelB) secretion signal sequence together with expression of a nucleic acid encoding the recombinant protein provides highly effective method for expressing high yields of recombinant protein without the requirement to harvest the recombinant cells from the fermentation culture and subsequently expose the cells to mechanical or enzymatical lysis so as to release the recombinant protein. When the T4 phage lysozyme gene is cloned behind a tightly-controlled promoter, such as araB, periplasmic accumulation of the T4 phage lysozyme may be induced by the addition of an inducer (such as arabinose) at an appropriate time in the fermentation process. Expression of the T4 phage lysozyme with a PelB signal sequence, facilitates the translocation T4 lysozyme to the periplasmic space where the enzyme degrades the peptidoglycan layer resulting in a morphological change in the cell that results in formation of a spheroplast.

The recombinant protein gene e.g., vitronectin gene, inorganic pyrophosphatase gene, MMuLV-RT gene, ribonuclease inhibitor gene, or 2-O-methyltransferase gene, is cloned under the control of an inducible promotor, such as PT7, lacUV5 or Ptac, expression of the polymerase/protein may be induced by addition of an inducer such as IPTG, at an appropriate time in the fermentation process. By placing the nucleic acid expression of the recombinant peptide (e.g., vitronectin, inorganic pyrophosphatase, MMuLV-RT, ribonuclease inhibitor or 2-O-methyltransferase) and the T4 phage lysozyme under separate promoter control, their expression can independently regulate the production of each protein during fermentation. The inventors have surprisingly demonstrated that the T4 lysozyme induced spheroplasts to secrete Taq or T7 polymerase and/or various other recombinant proteins e.g., vitronectin, inorganic pyrophosphatase, MMuLV-RT, ribonuclease inhibitor and 2-O-methyltransferase, into the culture supernatant without the need for a secretion signal sequence. The inventors surprisingly observed that recombinant expression of recombinant protein, e.g., vitronectin, inorganic pyrophosphatase, MMuLV-RT, ribonuclease inhibitor or 2-O-methyltransferase, has no detrimental impact on the host cell. In fact, the inventors have demonstrated that the induced spheroplasts were able to de novo express the recombinant protein, e.g., vitronectin, inorganic pyrophosphatase, MMuLV-RT, ribonuclease inhibitor or 2-O-methyltransferase for at least 4 hours (or at least 2 hours or at least 5 hours in some cases) after induction of T4 lysozyme.

Moreover, the inventors have unexpectedly demonstrated that the resulting fermented cell culture can be successfully clarified using a clarification filter having an unusually large pore size that provides a retention range of at least about 0.4 μm. This is wholly unexpected in as the established primary clarification approaches recommended for E. coli rely on tangential flow filtration using a filter that has a pore size of <0.2 μm together with centrifugation to remove cellular debris.

In addition, the inventors have advantageously demonstrated that fermented cell cultures of spheroplast can be efficiently clarified without the requirement for centrifugation. The removal of the requirement for a centrifugation step allows the fermentation and clarification to be performed in a closed system, thereby removing the risk of external contamination

Thus the combination of E. coli transformed with an expression system that allows permeabilization of the cell, in combination with a clarification process that does not require centrifugation, not only reduces the risk of contamination during protein production but additionally eliminates a number of subsequent downstream processing steps from the production including: cell harvesting, mechanical and or enzymatic cell lysis, cell debris separation, nucleic precipitation followed by separation using filtration or centrifugation. Thus the claimed methods provide high protein yields at high cell density and increased scale. Additionally, the elimination of the need to lyse the host cells and associated centrifugation steps, reduces large-scale processing time than with conventional lysis based methods.

### Expression System

The method of the invention uses an expression system. The system comprises i) a nucleic acid encoding a recombinant protein operably linked to a first inducible promotor; and ii) a nucleic acid encoding a T4 lysozyme protein in frame with a nucleic acid encoding PelB secretory signal peptide operably linked to a second inducible promotor, wherein the protein may be, for example, vitronectin, inorganic pyrophosphatase, MMuLV-RT, ribonuclease inhibitor or 2-O-methyltransferase.

As used herein “vitronectin” or “VTN” refers to a glycoprotein of 75 kD that is reported to bind to various biological ligands and play roles in tissue remodeling. Vitronectin may be of human origin and may have the sequence defined by NCBI sequence ID: AAH05046.1. Accordingly, vitronectin may comprise the sequence of SEQ ID NO: 2.

As used herein “inorganic pyrophosphatase” or “IPP” refers to an enzyme catalyzes the hydrolysis of inorganic pyrophosphate to form orthophosphate. Inorganic pyrophosphatase may pertain to the following: GeneBank: X13253.1, 99% identity, protein sequence: NCBI Reference Sequence: CAA31629.1, protein name: Ribonuclease Inhibitor. Accordingly, inorganic pyrophosphatase may comprise the sequence of SEQ ID NO: 3.

As used herein “MMuLV-RT”, “Moloney Murine Leukemia Virus Reverse Transcriptase”, or “MuLV-RT” refers to a DNA polymerase that synthesizes a complementary DNA strand from single-stranded RNA, DNA, or an RNA:DNA hybrid. MMuLV-RT protein may comprise the sequence of SEQ ID NO: 4.

As used herein “ribonuclease inhibitor” or “RI” refers to an enzyme that inhibit the activity of RNases. The ribonuclease inhibitor may be derived from Sus scrofa gene RNH1. The ribonuclease inhibitor may be optimised for expression in E. coli. The ribonuclease inhibitor may pertain to the following: NCBI Sequence ID: 445517, protein name: ribonuclease/angiogenin inhibitor 1 [Sus scrofa (pig)], protein sequence ID: XP_020938200.1. Accordingly, the ribonuclease inhibitor may comprise the sequence of SEQ ID NO:5.

As used herein “2-O-methyltransferase” or “OMT” refers to an enzyme that adds a methyl group at the 2′-O position of the first nucleotide adjacent to the cap structure at the 5′ end of the RNA. The 2-O-methyltransferase may be derived from Vaccinia virus gene vp39. The 2-O-methyltransferase may be optimised for expression in E. coli. The 2-O-methyltransferase may pertain to the following: NCBI Sequence ID: AGJ91263.1, protein name: multifunctional Poly-A polymerase-small subunit VP39 [Vaccinia virus]. Accordingly, the 2-O-methyltransferase may comprise the sequence of SEQ ID NO:6.

As used herein, “T4-lysozyme”, “E protein”, or “gpe” refers to a cytoplasmic muramidase that facilitates lysis of T4 phage-infected bacterial cells, thereby releasing replicated phage particles, (Tsugita and Inouye, J. Mol. Biol., 37: 201-12 (1968); Tsugita and Inouye, J. Biol. Chem., 243: 391-97 (1968), Uniprot accession: D9IEF7) or a mutant, derivative or fragment thereof having DNA polymerase activity It is encoded by gene e of T4 bacteriophage and hydrolyzes bonds between N-acetylglucosamine and N-acetylmuramic acid residues in the rigid peptidoglycan layer of the E. coli cell envelope. The enzyme is a single polypeptide chain of a molecular weight of 18.3 kDa. It is approximately 250-fold more active than HEW-lysozyme against bacterial peptidoglycan (Matthews et al., J. Mol. Biol., 147: 545-558 (1981)). The optimal pH for T4-lysozyme enzyme activity is 7.3, versus 9 for HEW-lysozyme. (The Worthington Manual; pp 219-221). The T4 enzyme may have the sequence defined by NCBI Reference Sequence: NP_049736.1. The T4 enzyme may comprise the sequence of SEQ ID NO: 1.

As used herein the term “recombinant” is intended to refer to proteins that are designed, engineered, prepared, expressed, created, manufactured, and/or or isolated by recombinant means, such as polypeptides expressed using a recombinant expression vector transfected into a host cell. The terms “recombinant protein” and “recombinant polypeptide” are considered as synonyms and used interchangeably. The recombinant protein may be any protein. In some embodiments the recombinant protein is a glycoprotein, a phosphatase, a polymerase, a nuclease, a transferase, a ligase, a glycosylase or a growth factor. In some embodiments the recombinant protein is selected from Vitronectin (VTN), pyrophosphatase (IPP), Moloney murine leukemia virus reverse transcriptase gene (MMLV RT), ribonuclease, Vaccinia virus 2-O-methyltransferase (OMT), Cas9, Eam11041 restriction endonuclease, Taq DNA polymerase, Klenow fragment DNA polymerase, phi29 DNA polymerase, Basic fibroblast growth factor (bFGF), T7 RNA Polymerase, RNAse inhibitor, Uracil-DNA Glycosylase (UDG), Shrimp Alkaline Phosphatase (SAP), RNase Inhibitor, reverse transcriptase (RT), Alkaline Phosphatase (AP), T4 RNA ligase, Uracil-DNA glycosylase, growth arrest and DNA damage-inducible protein GADD34. In some embodiments the recombinant protein is selected from Vitronectin (VTN), pyrophosphatase (IPP), Moloney murine leukemia virus reverse transcriptase (MuLV RT), ribonuclease, Vaccinia virus capping enzyme, 2-O-methyltransferase (OMT), Cas enzymes, such as Cas9, a type IIS restriction endonuclease, for example a type IIS restriction endonuclease from Enterobacter Amnigenus, such as Eam11041 restriction endonuclease having a cut site CTCTTC(1/4)\hat{ }, Taq DNA polymerase, Basic fibroblast growth factor (bFGF), T7 RNA Polymerase, or any inactive or inert protein fragment that may be used in the enzyme preparations as stabilizer, or as protein size standard for use in gel electrophoresis experiments. In some embodiments, the recombinant protein may be a heterologous protein. The term “heterologous” when used with reference to portions of a protein, indicates that the protein comprises two or more domains that are not found in the same relationship to each other in nature. In some embodiments, the recombinant protein contains two or more domains from unrelated proteins. In some embodiments, the recombinant protein comprise mutations (substitutions, insertions and/or deletions) compared to a known wild-type amino acid sequence. In some embodiments, the recombinant protein is a functional variant derived from a known wild-type polypeptide. For example the variant may be a functional variant of S. pyogenes Cas9 protein found in the SwissProt database under accession number Q99ZW2. In some embodiments, the recombinant protein is a functional variant derived from the polypeptide of SEQ ID NO: 1, 2, 3, 4, 5, or 6. For example the variant may be a functional variant of vitronectin, inorganic pyrophosphatase, MMuLV-RT, ribonuclease inhibitor or 2-O-methyltransferase. In some embodiments the polypeptide sequence of the functional variant is least 70%, 75%, 80%, 82%, 84%, 85%, 86%, 87%, 88%, 89%, 90%, 91%, 92%, 93%, 94%, 95%, 96%, 97%, 98%, 99%, identical to known wild-type polypeptide, such as to any of the sequences of SEQ ID NO: 1 to 6. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5. Alternatively, the percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

Expression systems for use in the method of the invention can be formed using methods that are well known in the art. In general, a cDNA or genomic DNA encoding the recombinant protein such as vitronectin, inorganic pyrophosphatase, MMuLV-RT, ribonuclease inhibitor, 2-O-methyltransferase or T4 lysozyme is suitably inserted into a replicable vector for expression in the E. coli under the control of a suitable promoter for E. coli. Suitable vectors are well known in the art, and the choice of the appropriate vector will be influenced by the size of the nucleic acid to be inserted into the vector and the host cell to be transformed with the vector. Each vector contains various components. For example, vectors for bacterial transformation will include a signal sequence for the recombinant protein and will also include a promoter for the recombinant protein. They preferably include an origin of replication and one or more marker genes.

Preferably, vectors are derived from species compatible with the host cell are used in connection with bacterial hosts, i.e. a vector compatible with E. coli. Preferably, the vector contains a replication site, and markers that provide for phenotypic selection of transformed cells. Expression vectors for use in the invention preferably contain a nucleic acid sequence that enables the vector to replicate in one or more selected host cells. Such sequences are well known for a variety of bacteria.

Expression vectors for use in the invention preferably contain a selection gene or selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors for use in the invention contain an inducible promoter that is recognized by the host bacterial organism and is operably linked to the nucleic acid encoding the polypeptide of interest, i.e. the recombinant protein e.g., vitronectin, inorganic pyrophosphatase, MMuLV-RT, ribonuclease inhibitor, 2-O-methyltransferase or T4 lysozyme.

Inducible promoters suitable for use with bacterial hosts such as E. coli include the β-lactamase and lactose promoter systems (Chang et al., Nature, 275: 615 (1978); Goeddel et al., Nature, 281: 544 (1979)), the arabinose promoter system, including the araBAD promoter (Guzman et al., J. Bacteriol., 174: 7716-7728 (1992); Guzman et al., J. Bacteriol., 177: 4121-4130 (1995); Siegele and Hu, Proc. Natl. Acad. Sci. USA, 94: 8168-8172 (1997)), the rhamnose promoter (Haldimann et al., J. Bacteriol., 180: 1277-1286 (1998)), the alkaline phosphatase promoter, a tryptophan (trp) promoter system (Goeddel, Nucleic Acids Res., 8: 4057 (1980) and EP 36,776), the PLtetO-1 and Plac/ara-1 promoters (Lutz and Bujard, Nucleic Acids Res., 25: 1203-1210 (1997)), and hybrid promoters such as the tac promoter (deBoer et al., Proc. Natl. Acad. Sci. USA, 80: 21-25 (1983)).

As used herein, “inducible promoter” refers to a promoter that directs transcription at an increased or decreased rate upon binding of a transcription factor.

As used herein a “transcription factor” refers to any factors that can bind to a regulatory or control region of a promoter and thereby effect transcription. The synthesis or the promoter-binding ability of a transcription factor within the host cell can be controlled by exposing the host to an “inducer” or removing a “repressor” from the host cell medium. Accordingly, to regulate expression of an inducible promoter, an inducer is added or a repressor removed from the growth medium of the host cell.

As used herein, the phrase “induce expression” means to increase the amount of transcription from specific genes by exposure of the cells containing such genes to an effector or inducer.

As used herein an “inducer” is a chemical or physical agent which, when given to a population of cells, will increase the amount of transcription from specific genes. These can be small molecules whose effects are specific to particular operons or groups of genes, and can include sugars, alcohol, metal ions, hormones, heat, cold, and the like. For example, isopropylthio-p-galactoside (IPTG) and lactose are inducers of the lac UV5 promoter, and L-arabinose is a suitable inducer of the arabinose (araB) promoter.

As used herein an “inducible promoter” is a promoter that direct transcription at an increased or decreased rate upon binding of a transcription factor.

As used herein “under control” refers to a nucleic acid sequence operably linked into a functional relationship with another nucleic acid sequence. For example a promoter or enhancer is operably linked to a coding sequence if it affects the transcription of the sequence. This requires that the DNA sequences being linked are contiguous and, in the case of a secretory peptides in reading frame.

Suitably, E. coli is transformed using pBR322, a plasmid derived from an E. coli species. See, e.g., Bolivar et al., Gene, 2: 95 (1977). pBR322 contains genes conferring ampicillin and tetracycline resistance for identifying transformed cells. The pBR322 plasmid, or other microbial plasmid or phage, also generally contains, or is modified to contain, promoters that can be used by the bacterial organism for expression of the selectable marker genes. The expression system of the invention comprises a nucleic acid encoding a recombinant protein operably linked to a first inducible promotor.

The expression system of the invention also comprises a nucleic acid encoding a T4 lysozyme protein in frame with a nucleic acid encoding PelB secretory signal peptide operably linked to a second inducible promotor.

As used herein, “secretory signal peptide” or “signal peptide” refers to a peptide that can be used to secrete a recombinant protein into the periplasm of a host bacteria. The signal for the heterologous polypeptide may be endogenous to the bacteria, or they may be exogenous, including signals native to the polypeptide being produced in the host bacteria. In general, the signal sequence may be a component of the vector, or it may be a part of the polypeptide DNA that is inserted into the vector. Preferably, the vector contains a nucleic acid encoding a signal sequence at the N-terminus of the T4 lysozyme protein. Preferably, the secretory signal peptide is pectate lyase B (PelB). In an embodiment, the PelB sequence may be from Erwinia spp. Accordingly, disclosed herein is gpe with a pelb signal sequence that comprises the sequence of SEQ ID NO:1. The secretion signal sequence may directly linked to the nucleotide sequence encoding the recombinant protein, e.g., vitronectin, inorganic pyrophosphatase, MMuLV-RT, ribonuclease inhibitor, 2-O-methyltransferase. Alternatively, there may be a spacer between the secretory signal peptide and the nucleotide sequence encoding the recombinant protein, e.g., vitronectin, inorganic pyrophosphatase, MMuLV-RT, ribonuclease inhibitor, 2-O-methyltransferase. The spacer may comprise more or less than 9 nucleotides such as, for example, between 5 and 20 nucleotides.

Construction of vectors containing one or more of the above described components uses ligation techniques well known in the art. DNA fragments are cleaved, and re-ligated in the form required to generate suitable vectors.

Confirmation that constructed plasmids and vectors contain the correct sequences can be achieved by transforming E. coli K12 strain 294 (ATCC 31,446) or other appropriate strains, and selecting transformants by ampicillin or tetracycline resistance.

In embodiments the expression system comprises a first vector that expresses the recombinant protein under the control of a first inducible promotor. In embodiments the first inducible promotor is a T7 promotor or tac promotor. In embodiments the first vector is a pET vector, such as pET29, pET21, pET21b, and the inducible promoter is a T7 promotor. In one embodiment the vector is a pLATE vector, such as pLATE11, 31, 51 or 52 and the inducible promoter is a T7 promotor.

In embodiments the expression system comprises a second vector that expresses T4 page lysozyme having an N-terminal PelB signal sequence under the control of a second inducible promotor. In embodiments, the second inducible promotor is a T7 promotor or araB promotor. In embodiments the second vector is a pACYC184 vector. In one embodiment the vector is pAra-pelB-gpe. In one embodiment the vector is pACYC184-PT7-pelB-gpe-lacl.

In one embodiment the vector expresses vitronectin under the control of the IPTG inducible pT7 promoter or the IPTG inducible lacUV5 promoter. Preferably the vector is a pET29 vector. In one embodiment the vector is a pBR322 vector. In one embodiment the vector is a pET29—optVTN as disclosed herein.

In one embodiment the vector expresses T4 phage lysozyme having an N-terminal PelB signal sequence under the control of T7 polymerase promoter. In one embodiment the vector is pLATE11 (Cat. K1241, Thermo Fisher). In one embodiment, the vector is pACYC184-PT7-pelB-gpe-lacl as disclosed herein. In one embodiment the T7 polymerase promoter has two flanking lac operator sequences to ensure tight control of gene expression, and may further have one or more of (i) a Ptet promoter to reduce basal expression from the T7 promoter, (ii) a T7 terminator which terminates transcription from the T7 promoter and (iii) a lac repressor which ensures tight control of basal expression from the T7 promoter. In one embodiment the T7 promoter may be subcloned into a pACYC184 vector that has a p15A origin of replication and is compatible with plasmids harboring ColE1 ori.

In one embodiment the vector expresses T4 page lysozyme having an N-terminal PelB signal sequence under the control of the araB promoter. In one embodiment the vector is a pBR322 vector. In one embodiment the vector is a pET29 vector. In one embodiment the vector is pLysS-lacl-PcsP-pelB-gpe, where lacl-PcsP is scold includible promoter csp in tandem with IPTG inducible operon lacl. In one embodiment the vector is pACYC184-lacl-Pcsp-PelB-gpe.

In one embodiment the vector expresses inorganic pyrophosphatase under the control of the IPTG inducible pT7 promoter or the IPTG inducible lacUV5 promoter. Preferably the vector is a pET21b vector. In one embodiment the vector is a pBR322 vector. In one embodiment the vector is a pET21b-ppa-Kn as disclosed herein.

In one embodiment the vector expresses T4 page lysozyme having an N-terminal PelB signal sequence under the control of the araB promoter. For example, the T4 bacteriophage lysozyme gene gpe with signal peptide of pelB may be cloned into pAra vector (Thermo Fisher) under the araB promoter (plasmid pAra-pelB-gpe). The plasmid pAra-pelB-gpe carries an origin of replication derived from pACYC and a chloramphenicol-resistance gene gene which allows the use of E. coli expression systems containing ColE1-type plasmids. Accordingly, in one embodiment the vector is pAra-pelB-gpe. In one embodiment the vector is a pAra vector. In one embodiment the vector is a pBR322 vector. In one embodiment the vector is a pET29 vector. In one embodiment the vector is pLysS-lacl-PcsP-pelB-gpe, where lacl-PcsP is scold includible promoter csp in tandem with IPTG inducible operon lacl. In one embodiment the vector is pACYC184-lacl-Pcsp-PelB-gpe.

In one embodiment the vector expresses MMuLV-RT under the control of the IPTG inducible Ptac promoter or the IPTG inducible lacUV5 promoter. Preferably the vector is a pBR322 vector. In one embodiment the vector is a pET29 vector. In one embodiment the vector is a pMuLV-RT-Km as disclosed herein.

In one embodiment the vector expresses T4 page lysozyme having an N-terminal PelB signal sequence under the control of the araB promoter. For example, the T4 bacteriophage lysozyme gene gpe with signal peptide of pelB was cloned in to pAra vector (Thermo Fisher) under araB promoter (plasmid pAra-pelB-gpe). The plasmid pAra-pelB-gpe carries an origin of replication derived from pACYC and a chloramphenicol-resistance gene gene which allows the use of E. coli expression systems containing ColE1-type plasmids. Accordingly, in one embodiment the vector is pAra-pelB-gpe. In one embodiment the vector is a pAra. In one embodiment the vector is a pBR322 vector. In one embodiment the vector is a pET29 vector. In one embodiment the vector is pLysS-lacl-PcsP-pelB-gpe, where lacl-PcsP is scold includible promoter csp in tandem with IPTG inducible operon lacl. In one embodiment the vector is pACYC184-lacl-Pcsp-PelB-gpe.

In one embodiment the vector expresses ribonuclease inhibitor under the control of the IPTG inducible pT7 promoter or the IPTG inducible lacUV5 promoter. Preferably the vector is a pET vector. In one embodiment the vector is a pET29 vector. In one embodiment the vector is a pET29-optRNAseOUT as disclosed herein.

In one embodiment the vector expresses T4 page lysozyme having an N-terminal PelB signal sequence under the control of the araB promoter. For example, the T4 bacteriophage lysozyme gene gpe with signal peptide of pelB may be cloned into pAra vector (Thermo Fisher) under the araB promoter (plasmid pAra-pelB-gpe). The plasmid pAra-pelB-gpe carries an origin of replication derived from pACYC and a chloramphenicol-resistance gene which allows the use of E. coli expression systems containing ColE1-type plasmids. Accordingly, in one embodiment the vector is pAra-pelB-gpe. In one embodiment the vector is a pAra vector. In one embodiment the vector is a pBR322 vector. In one embodiment the vector is a pET29 vector. In one embodiment the vector is pLysS-lacl-PcsP-pelB-gpe, where lacl-PcsP is scold includible promoter csp in tandem with IPTG inducible operon lacl. In one embodiment the vector is pACYC184-lacl-Pcsp-PelB-gpe.

In one embodiment the vector expresses 2-O-methyltransferase under the control of the IPTG inducible pT7 promoter or the IPTG inducible lacUV5 promoter. Preferably the vector is a pLATE31 vector. In one embodiment the vector is a pBR322 vector. In one embodiment the vector is a pLATE31-VP39delta32-KnR as disclosed herein.

In one embodiment the vector expresses T4 page lysozyme having an N-terminal PelB signal sequence under the control of the araB promoter. For example, the T4 bacteriophage lysozyme gene gpe with signal peptide of pelB may be cloned into tight controlled pLATE11 vector (Cat. K1241, Thermo Fisher) to reduce the expression of lysis genes in uninduced cells—the fragment containing the transcription terminator rrnBT1-T2 prevents basal gene expression from vector derived promoter-like elements, the T7 RNA polymerase promoter, which drives transcription of the cloned gpe gene, and two flanking lac operator sequences ensure tight control of gene expression, the Ptet promoter reduces basal expression from the T7 promoter, the T7 terminator terminates transcription from the T7 promoter and the lac repressor which ensures tight control of basal expression from the T7 promoter may be subcloned into the pACYC184 vector that has a p15A origin of replication and is compatible with plasmids harboring ColE1 on (plasmid pACYC184-PT7-pelB-gpe-lacl). Accordingly, in one embodiment, the vector is pACYC184-PT7-pelB-gpe-lacl. In one embodiment the vector is pLATE11. In one embodiment the vector is a pBR322 vector. In one embodiment the vector is a pET29 vector. In one embodiment the vector is pLysS-lacl-PcsP-pelB-gpe, where lacl-PcsP is scold includible promoter csp in tandem with IPTG inducible operon lacl. In one embodiment the vector is pACYC184-lacl-Pcsp-PelB-gpe.

### Host Cells

The method of the invention uses a host cell, i.e. E. coli transformed or transfected with the above described expression systems.

As used herein “transformed” or “transformation” refers to introducing DNA into an organism so that the DNA is replicable, either as an extrachromosomal element or as chromosomal integration. Depending on the host cell used, transformation is done using standard techniques appropriate to such cells.

Recombinant cells for use in the methods of the invention may be prepared by recombinant DNA techniques that are familiar to one or ordinary skill in the art (see e.g., Kotewicz, M. L., et al., Nucl. Acids Res. 16:265 (1988); Soltis, D. A., and Skalka, A. M., Proc. Natl. Acad. Sci. USA 85:3372-3376 (1988)). Such recombinant cells may be grown according to standard microbiological techniques, using culture media and incubation conditions suitable for growing active cultures of the particular species that are well-known to one of ordinary skill in the art (see, e.g., Brock, T. D., and Freeze, H., J. Bacteriol. 98(1):289-297 (1969); Oshima, T., and Imahori, K., Int. J. Syst. Bacteriol. 24(1):102-112 (1974)).

Transformation means introducing DNA into an organism so that the DNA is replicable, either as an extrachromosomal element or as chromosomal integration. Depending on the host cell used, transformation is done using standard techniques appropriate to such cells. The calcium treatment employing calcium chloride, as described in Sambrook et al., Molecular Cloning: A Laboratory Manual (New York: Cold Spring Harbor Laboratory Press, 1989), is generally used for bacterial cells that contain substantial cell-wall barriers. Another method for transformation employs polyethylene glycol/DMSO, as described in Chung and Miller, Nucleic Acids Res., 16: 3580 (1988). Yet another method is the use of the technique termed electroporation.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers.

As used herein “Escherichia coli” or “E. coli” refers to a Gram-negative, facultative anaerobic, rod-shaped, coliform bacterium of the genus Escherichia. E. coli host strains for recombinant DNA product fermentations are well known in the art and include E. coli B such as BL21 (DE3), or its derivatives, such as E. coli W3110 (ATCC 27,325), E. coli 294 (ATCC 31,446), and E. coli X1776 (ATCC 31,537). In the methods of the invention E. coli B strain, JS007 strain or JS008 strain may be a preferred host.

In the method of the invention, the E. coli may be transformed with one or two expression vectors containing the nucleic acid encoding a recombinant protein and the nucleic acid encoding the T4 lysozyme protein. In one embodiment protein, the bacterial cells are transformed with two vectors respectively containing the nucleic acid encoding the recombinant protein and the nucleic acid encoding T4 lysozyme protein. In an alternative embodiment, the nucleic acid encoding the nucleic acid encoding the recombinant protein and the nucleic acid encoding T4 lysozyme protein are contained on one vector with which the bacterial cells are transformed.

E. coli host cells are transformed with the above-described expression vector(s) of this invention and cultured in conventional nutrient media modified as appropriate for inducing the various promoters if induction is carried out.

In one embodiment the host cell is E. coli, preferably E. coli B strain, transformed with the vector expressing vitronectin under the control of the IPTG inducible PT7 or IPTG inducible lacUV5 promoter. The vector may be any suitable vector such as a pET29 or a pBR322 vector.

In one embodiment the host cell is E. coli, preferably E. coli B strain such as BL21(DE3), transformed with the vector expressing T4 page lysozyme having an N-terminal PelB signal sequence under the control of the PT7 or araB promoter. The vector may be any suitable vector such as a pBR322 vector.

In one embodiment the host cell is E. coli, preferably E. coli B strain, transformed with the vector expressing e.g., vitronectin or IPP under the control of the IPTG inducible promoter such as a PT7 or lacUV5 promoter and T4 phage lysozyme having an N-terminal PelB signal sequence under the control of a promoter such as a pT7 promoter or the araB promoter. The vector may be any suitable vector disclosed herein, such as a pBR322 vector.

In one embodiment the host cell is E. coli, preferably E. coli B strain or JS007 strain, transformed with the vector expressing MMuLV-RT under the control of the IPTG inducible PTac or IPTG inducible lacUV5 promoter. The vector may be any suitable vector such as a pBR322 vector.

In one embodiment the host cell is E. coli, preferably E. coli B strain such as BL21(DE3) or JS007 strain, transformed with the vector expressing T4 page lysozyme having an N-terminal PelB signal sequence under the control of the araB promoter. The vector may be any suitable vector such as a pBR322 vector.

In one embodiment the host cell is E. coli, preferably E. coli B strain or JS007 strain, transformed with the vector expressing MMuLV-RT under the control of the IPTG inducible promoter such as a PT7 or lacUV5 promoter and T4 phage lysozyme having an N-terminal PelB signal sequence under the control of the araB promoter. The vector may be any suitable vector disclosed herein, such as a pBR322 vector.

In one embodiment the host cell is E. coli, preferably E. coli B strain or E. coli JS008, transformed with the vector expressing ribonuclease inhibitor under the control of the IPTG inducible PT7 or IPTG inducible lacUV5 promoter. The vector may be any suitable vector such as a pET29 vector.

In one embodiment the host cell is E. coli, preferably E. coli B strain such as BL21(DE3) or preferably E. coli JS008, transformed with the vector expressing T4 page lysozyme having an N-terminal PelB signal sequence under the control of the araB promoter. The vector may be any suitable vector such as a pAra or a pBR322 vector.

In one embodiment the host cell is E. coli, preferably E. coli B strain or preferably E. coli JS008, transformed with the vector expressing ribonuclease inhibitor under the control of the IPTG inducible promoter such as a PT7 or lacUV5 promoter and T4 phage lysozyme having an N-terminal PelB signal sequence under the control of the araB promoter. The vector may be any suitable vector disclosed herein, such as a pET29 vector pAra vector or a pBR322 vector.

In one embodiment the host cell is E. coli, preferably E. coli B strain or E. coli JS007, transformed with the vector expressing 2-O-methyltransferase under the control of the IPTG inducible PT7 or IPTG inducible lacUV5 promoter. The vector may be any suitable vector such as a pLATE31 or a pBR322 vector.

In one embodiment the host cell is E. coli, preferably E. coli B strain such as BL21(DE3) or preferably E. coli JS007, transformed with the vector expressing T4 page lysozyme having an N-terminal PelB signal sequence under the control of the araB promoter. The vector may be any suitable vector such as a pLATE11 or a pBR322 vector.

In one embodiment the host cell is E. coli, preferably E. coli B strain or preferably E. coli JS007, transformed with the vector expressing 2-O-methyltransferase under the control of the IPTG inducible promoter such as a PT7 or lacUV5 promoter and T4 phage lysozyme having an N-terminal PelB signal sequence under the control of the araB promoter. The vector may be any suitable vector disclosed herein, such as a pACYC184 vector or a pBR322 vector.

### Cell Culture

The transformed host cells described above are fermented so as to express the nucleic acid encoding the recombinant protein (e.g., vitronectin, inorganic pyrophosphatase, MMuLV-RT, ribonuclease inhibitor, or 2-O-methyltransferase) and the nucleic acid encoding the T4-lysozyme.

As used herein, the terms “fermenting” and “culturing” are used interchangeably to refer to bulk growing cells in a growth medium. This includes an exponential phase, characterized by cell doubling, therefore bulk growth and a stationary phase, where the growth rate and death rate of cells is equal often due to a growth-limiting factor such as the depletion of an essential nutrient, and/or the formation of an inhibitory product such as an organic acid.

In the process of the invention expression each of the recombinant protein and T4-lysozyme of is controlled by induction of the associated promoters. Induction of the promotors for each of polymerase (or various proteins such as vitronectin, inorganic pyrophosphatase, MMuLV-RT, ribonuclease inhibitor, or 2-O-methyltransferase) and T4-lysozyme can be performed sequentially or simultaneously. The induction of expression of the nucleic acid encoding the recombinant protein and T4-lysozyme is preferably carried out by adding an inducer to the culture. Typically, the inducer is added after the cells are cultured until a certain optical density has been reached, e.g. an OD600 of 0.5, 1.0, 1.5, 2.0, 2.5, 3.0, 3.5, 4.0, 4.5, 5.0 or more. In one embodiment, the inducer for the T4-lysozyme is added simultaneously with the inducer for the recombinant protein. In one embodiment, the inducer for T4-lysozyme is added sequentially following induction of the recombinant protein e.g., as vitronectin, inorganic pyrophosphatase, MMuLV-RT, ribonuclease inhibitor, or 2-O-methyltransferase.

In one embodiment induction of the of the promotors for each of the recombinant protein and the T4-lysozyme is simultaneous. In this embodiment, the promoters for the T4-lysozyme and the polymerase encoding nucleic acid may be identical. While the promoters may be any suitable promoters for this purpose, preferably, the promoters for the recombinant protein and polymerase are IPTG inducible promoters, for example T7 promotors.

In one embodiment induction of the of the promotors for each of the recombinant protein and the T4-lysozyme is sequential and induction of the promotor for the T4-lysozyme commences about 1, 2, 3, 4, 5, 6, 7, 8, 9, 10 or more hours after induction of the promotor for the recombinant protein. In this embodiment, the promoters the T4-lysozyme encoding nucleic acid and the polymerase encoding nucleic acid must be different, such that the nucleic acid-encoded recombinant protein expression is induced before expression of nucleic acid-encoded T-4 lysozyme or at a much higher level, when the promoters are inducible. While the promoters may be any suitable promoters for this purpose, preferably, the promoters for the recombinant protein and polymerase are, respectively, arabinose promoter and IPTG inducible promoter.

When the inducer for the T4-lysozyme is added sequentially, the inducer is typically added after a desired amount of recombinant protein has accumulated (for example as determined by the optical density reaching a target amount observed in the past to correlate with the desired polypeptide accumulation). Typically, the induction of the T4 lysozyme takes place at a point in time post-inoculation about 75-90%, preferably about 80-90%, of the total fermentation process time. For example, induction of the T4-lysozyme promoter may take place at from about 30 hours, preferably 32 hours, up to about 36 hours post-inoculation of a 40-hour fermentation process. For example, induction of the T4-lysozyme promoter may take place at from about 1, 2, 3, 4, 5, 6, 7, 8 9, 10 or more hours post-induction of the recombinant protein (e.g., vitronectin, inorganic pyrophosphatase, MMuLV-RT, ribonuclease inhibitor, 2-O-methyltransferase) expression.

Induction of the T4-lysozyme permeabilizes the cell so as to form a spheroplast that secretes the recombinant protein (e.g., vitronectin, inorganic pyrophosphatase, MMuLV-RT, ribonuclease inhibitor, 2-O-methyltransferase). As used herein “permeabilizing” refers to making openings in the cell wall without totally removing it. A “spheroplast” is a cell in which the cell wall of a Gram-negative bacterium is partially lost and becomes spherical. Spheroplastization can be performed by treating Gram-negative bacteria with lysozyme, penicillin, or the like. Since the cell membrane of spheroplastized Gram-negative bacteria is partially lost, information on the interaction between the target protein and the polypeptide (electrophysiological change) can be analysed by an electrophysiological method. In addition, the same polypeptide is present in high concentration in the spheroplast of one cell, which is a favourable condition for measuring the activity of the polypeptide on the target protein. Typically, spheroplasts that retain substantially all the nucleic acids within the spheroplast while allowing intracellular proteins (including recombinant protein) to move across the spheroplast membrane.

Typically, the culturing step takes place under conditions of high cell density, that is, generally at a cell density of about 15 to 150 g dry weight/litre, preferably at least about 40, more preferably about 40-150, most preferably about 40 to 100. In addition, the culturing can be accomplished using any scale, even very large scales of 100,000 litres. Preferably, the scale is about 100 litres or greater, more preferably at least about 500 litres, and most preferably from about 500 litres to 100,000 litres.

E. coli cells used to produce the polypeptide of interest described in this invention are cultured in suitable media in which the promoters can be induced as described generally, e.g., in Sambrook et al., supra.

The terms “medium”, “cell culture medium” and “culture medium” and “fermentation medium” are interchangeably used herein and refer to a solution containing nutrients which are required for growing bacterial, i.e. E. coli cells. Typically, a cell culture medium provides essential and non-essential amino acids, vitamins, energy sources, lipids, and trace elements required by the cell for minimal growth and/or survival. Preferably, the medium is chemically defined in that all its components and their concentration are known. If the cells are cultured in fed-batch mode as described below, the term “culture medium” refers to both the basal medium and the feed medium, unless stated otherwise. The pH of the medium may be any pH from about 5-9, depending mainly on the host organism.

In order to accumulate the recombinant gene product, i.e. the recombinant protein such as vitronectin, inorganic pyrophosphatase, MMuLV-RT, ribonuclease inhibitor, or 2-O-methyltransferase, the host cell is cultured under conditions sufficient for accumulation of the gene product. Such conditions include, e.g., temperature, nutrient, and cell-density conditions that permit protein expression and accumulation by the cell. Moreover, such conditions are those under which the cell can perform basic cellular functions of transcription, translation, and passage of proteins from one cellular compartment to another for the secreted proteins, as are known to those skilled in the art.

Different strategies are available for cell culture, including batch culture, perfusion culture, continuous culture and fed-batch culture.

In one embodiment of the methods of the present invention the temperature is changed during the culture process from a first temperature to a second temperature, i.e. the temperature is actively downregulated. Preferably, the second temperature is lower than the first temperature. The first temperature may be 37° C.±0.2° C. The second temperature may be in the range of from 25° C. to 36° C., preferably it is in the range of 28° C. to 36° C., more preferably it is in the range of 28° C. to 32° C., even more preferably it is in the range of 29° C. to 31° C. The second temperature may be 29° C., 30° C. or 31° C. In one embodiment the first temperature is 37° C. and the second temperature is 30° C.

Gene expression may be measured in a sample directly, for example, by conventional northern blotting to quantitate the transcription of mRNA (Thomas, Proc. Natl. Acad. Sci. USA, 77: 5201-5205 (1980)).

In some embodiments, the bacteria are cultured in a bioreactor. As described herein the methods of the invention have been shown to be particularly applicable to large scale production, e.g., in a bioreactor or vessels over 1 L in volume. In some embodiments, the bioreactor is at least about 1 L in volume, at least about 5 L in volume, at least about 10 L in volume, at least about 15 L in volume, at least about 20 L in volume, at least about 30 L in volume, at least about 40 L in volume, at least about 50 L in volume, at least about 100 L in volume, at least about 200 L in volume, at least about 250 L in volume, at least about 500 L in volume, at least about 750 L in volume, at least about 1000 L in volume, at least about 1500 L in volume, at least about 2000 L in volume, at least about 2500 L in volume, at least about 3000 L in volume, at least about 3500 L in volume, at least about 4000 L in volume, at least about 5000 L in volume, at least about 7500 L in volume, at least about 10,000 L in volume, at least about 15,000 L in volume, at least about 20,000 L in volume, at least about 30,000 L in volume, at least about 50,000 L in volume, at least about 100,000 L in volume, at least about 150,000 L in volume, at least about 200,000 L in volume, at least about 250,000 L in volume, at least about 300,000 L in volume, at least about 350,000 L in volume, at least about 400,000 L in volume, at least about 450,000 L in volume or at least about 500,000 L in volume.

Since the recombinant protein (e.g., vitronectin, inorganic pyrophosphatase, MMuLV-RT, ribonuclease inhibitor, or 2-O-methyltransferase) is advantageously secreted into the cell culture supernatant during the cultivation process, the recombinant protein can be isolated at the end of the cultivation process by separating cell culture supernatant comprising the recombinant protein from the cells. The method advantageously removes the requirement to lyse the transformed cells in order to isolate the recombinant polymerase/protein. The presence of cell debris and release of nucleic acids and other molecules that could affect the quality of the recombinant protein product is thereby minimised

Harvest represents the end of fermentation/culture. Harvest may be at any time point during fermentation that is considered sufficient to end the fermentation process and recover the recombinant protein being expressed. Harvest may occur between 10 and 60 hours post induction of conditions to allow the expression of the recombinant protein. For example, harvest may occur between 15 and 40 hours post induction. At harvest, the fermented culture media will comprise cells that have undergone autolysis and membrane permeabilization. For example, about 50% or more of the cells in the harvest may have undergone autolysis. Alternatively, about 50%, 55%, 60%, 65%, 70%, 80% or 85% of the cells in the harvest have autolysed.

As used herein “cell lysis” and “lysis” refers to release of DNA from a cell due to the total disruption of the cell membrane. If DNA is lost from the cell, the cell can be classed as no longer viable, i.e. dead. In contrast, “membrane permeabilization” and “permeabilization” refer to disruption of the integrity of the cell membrane, that results of leaking of the recombinant product into the cell culture supernatant, without complete lysis of the cell membrane. The method of the invention allows for clarification of recombinant protein to be performed without total disruption of the cells. Specifically, the method of the invention provides for clarification of recombinant protein to be performed without mechanical disruption of the cell; without the addition of an exogenous enzyme that degrades the cell wall; without transformation of the cell with an expression vectors containing the nucleic acid encoding an exogenous enzyme that degrades the cytoplasmic membrane, e.g. a T4 holin protein. Accordingly, in embodiments the method of the invention does not include any of the following: (i) mechanical disruption of the cells; (ii) addition of an exogenous enzyme that degrades the cell wall; and (iii) transformation of the cell with an expression vectors containing the nucleic acid encoding an exogenous enzyme that degrades the cytoplasmic membrane, e.g. a T4 holin protein.

Techniques are known in the art to monitor both recombinant product leaking and cell lysis. The general approach for both is the detection of marker molecules, e.g. extracellular product for leakiness or DNA for lysis. Techniques for measuring leakiness include Photometry (Dietzsch, J. Biotechnol., 163, pp. 362-370 (2013); Wurm, Eng. Life Sci., 17 pp. 215-222 (2017)), and High Performance Liquid Chromatography (Amand, Biotechnol. Prog., 30, pp. 249-255 (2014); Kaiser Eng. Life Sci., 8 pp. 132-138 (2008);). Techniques for measuring lysis include Colorimetric assays in manually operated photometers (at-line) or paired with liquid handling systems (at/on-line) (Rajamanickam, Anal. Bioanal. Chem., 409, pp. 667-671 (2017)) and Infrared-/Raman-Spectroscopy (Abu-Absi, Pharm. Bioprocess., 2, pp. 267-284 (2014); Buckley, Appl. Spectrosc., 71, pp. 1085-1116 (2017); ClaBen, Anal. Bioanal. Chem., 409 pp. 651-666 (2017); Sales, Biotechnol. Prog., 33 pp. 285-298 (2017)). Autolysis may be indirectly determined by DNA concentration in a clarified harvest, or by capacitance.

Secreted recombinant proteins are susceptible to instability in the cell culture, for example aggregate and misfolding after secretion/cell lysis. In certain embodiments, a stabilizing agent is added to the cell culture. The stabilizing agent may be added before, during or after fermentation. Examples of suitable stabilizing agents include potassium chloride, DTT, sodium chloride, trehalose, sucrose, glycine betaine, mannitol, potassium citrate, CuC₁₂, proline, xylitol, NDSB 201, CTAB, K₂PO₄ Na₂SO₄, and Brij 35. In certain embodiments, the stabilizing additive can include amino acids such as arginine.

In certain embodiments a nucleic acid inactivation agent (such as benzonase) may be used to remove the free nucleic acid (DNA and/or RNA) from the cell culture. The nucleic acid inactivation agent may be added before, during or after fermentation. Preferably addition of the nucleic acid inactivation agent is not followed by centrifugation prior to clarification of the cell culture. Thus, any method of the invention may comprise the steps of (i) addition of a nucleic acid (e.g. DNA) inactivation agent (such as benzonase) to the cell culture in the absence of a subsequent centrifugation step.

### Clarification

In accordance with the methods of the invention, the autolysed and or permeabilized cells are separated from the culture media prior to recovery and purification of the product from the extracellular medium by clarification.

As used herein the terms “clarify”, “clarification”, “clarification step” refers to a process, containing one or more steps, used to remove solid impurities from the cell culture in order to obtain a liquid crude recombinant protein containing filtrate.

The efficiency of the clarification step is crucial to facilitate the subsequent downstream processing steps of purification of the biomolecule of interest. Parameters that may impact the clarification step include the total cell concentration, the cell viability, the initial turbidity of the cell culture to clarify, the concentration of biomolecule produced by the cultured cells and the size of the cell debris. As used herein, the term “Total cell concentration” (TCC) refers to the number of cells in a given volume of culture. As used herein the term “Viable cell concentration” (VCC) refers to the number of live cells in a given volume of culture, as determined by standard viability assays known in the art. As used herein the term “viability” refers to the percentage of living cells. Generally, the greater the TCC the greater the biomass to be removed from the cell culture and the greater the impact on the clarification. As used herein, the term “turbidity” refers to the cloudiness or haziness of a liquid caused by large numbers of individual particles. In particular, the turbidity indicates the amount of material and small particles inside a liquid capable of light diffusion. Generally, the turbidity of a cell culture correlates with the presence of cells, cell debris, nucleic acids and host cell proteins (HCP) in the culture, in addition to the recombinant protein.

A clarification step reduces of the initial turbidity of the fermented cell culture to a lower turbidity of the clarified cell culture to obtain a clarified cell culture with the highest concentration of the biomolecule of interest and smaller presence of other cell culture material.

In one embodiment the initial turbidity of the fermented cell culture is greater than about 10,000 NTU, about 20,000 NTU, about 30,000 NTU. In some embodiments the initial turbidity of the fermented cell culture is between about 10,000 NTU and about 150, 000 NTU, more specifically comprised between about 30,000 NTU and about 100,000 NTU.

In one embodiment the turbidity of the clarified cell culture is equal to or less than about 40, 30, 25, or 20 NTU. Preferably the turbidity of the clarified cell culture is equal to or less than about 20 NTU

For a clarification method to be efficient, it is also important that the throughput is maximized. As used herein the terms “throughput” or “loading capacity” or “capacity” are interchangeable and indicate the volume clarified by a clarification operational unit, for instance the volume filtered through a filter, more particularly, the volume normalized by filter's area (L/m²).

The cell culture clarification of the invention comprises a primary clarification step. As used herein the terms “primary clarification” refers to the removal of large particles such as whole cells and cell debris. In embodiments the primary clarification may be followed by a secondary clarification step. As used herein the term “secondary clarification” typically refers to the removal of smaller particle, e.g. particles smaller than whole cells. Each of the primary and secondary clarification steps employs as the clarification operational unit a filter.

In a preferred embodiment the primary clarification step and/or secondary clarification step is microfiltration. Generally, microfiltration is used to separate particles having a size of 0.1-10 μm from a solution. It is generally used to separate a polymer having a molecular weight of 1×10⁵ g/mol. In addition, microfiltration may be used to remove sediments, protozoan animals, large bacteria, etc. In the present invention, microfiltration may be easily used to remove whole cells and cell debris. Generally, the microfiltration process is performed using a pressure pump or a vacuum pump at a velocity of 0.1-5 m/s, preferably 1-3 m/s, and a pressure of 50-600 kPa, preferably 100-400 kPa.

In a more preferred embodiment, the primary clarification step and/or secondary clarification step is depth filtration. As used herein, the term “depth filtration” refers to a technology that exploits filters with a certain porosity to retain particles of a medium throughout the filter, rather than just on the filter surface. Depth filters are typically composed of cellulose fibers or synthetic polymeric fibers like polyacrylic or polystyrene. These fibers form a three-dimensional network with a certain porosity. A depth filter can be suitable for both primary and/or secondary recovery, or for primary or secondary recovery only. Filters suitable for primary and/or secondary recovery are also known as “single filters”. Single filters can be applied alone to carried out both the primary and the secondary recovery, or they can be used as filters for primary recovery and coupled with the subsequent use of a filter for secondary recovery. In certain embodiments that depth filter is positively charged. The interactions between the positively charged filter and colloidal particles in the fermented cell culture advantageously allows for the removal of such small contaminants whereas bigger particles are trapped by the porosity of the filter matrix.

The filter may be composed of a variety of materials well known in the art. Such materials may include, but are not limited to, polymeric materials such as nylon, cellulose materials (e.g., cellulose acetate), polyvinylidene fluoride (PVDF), polyethersulfone, polyamide, polysulfone, polypropylene, and polyethylene terephthalate. In certain embodiments, filters useful for clarification of cell lysates may include, but are not limited to Stax™ depth filter systems (Pall Life Sciences) ULTIPLEAT PROFILE™ filters (Pall Corporation, Port Washington, NY), SUPOR™ membrane filters (Pall Corporation, Port Washington, NY). Commercially available depth filters include Stax™ depth filter systems (Pall Life Sciences) Millistak+ Pod depth filter system, XOHC media (Millipore Corporation), Zeta Plus™ Depth Filter (3M Purification Inc.), etc.

In embodiments, the primary clarification is performed by a first filter, having a pore size that provides a retention range of at least about 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0 μm (micrometre) or more. In another embodiment the first filter has a pore size that provides a retention range of from about 0.4 μm to about 20.0 μm. In another embodiment the first filter has a pore size that provides a retention range of from about 0.4 μm to about 9.0 μm. In another embodiment the first filter has a pore size that provides a retention range of from about 0.4 μm to about 3.0 μm. In another embodiment the first filter has a pore size that provides a retention range of from about 0.4 μm to about 0.8 μm. In another embodiment the first filter has a pore size that provides a retention range of from about 1.0 μm to about 3.0 μm. In another embodiment the first filter has a pore size that provides a retention range of from about 1.0 μm to about 20.0 μm. In another embodiment the first filter has a pore size that provides a retention range of from about 1.0 μm to about 9.0 μm. In another embodiment the first filter has a pore size that provides a retention range of from about 4.0 μm to about 20.0 μm. In another embodiment the first filter has a pore size that provides a retention range of from about 4.0 μm to about 9.0 μm. In a particular embodiment the first clarification step is carried out by a first depth filtration.

Efficiency of clarification can be improved by maximizing filter throughput. As used herein the terms “throughput” or “loading capacity” or “capacity” are interchangeable and indicate the volume clarified by a clarification operational unit, for instance the volume filtered through a filter, more particularly, the volume normalized by filter's area (L/m²).

In one embodiment, the primary clarification step has a maximum throughput of at least about 50, 60, 70, 80, 90, 100, 150,200, 250, 300, 350, 400, 450, 500, 550, 600, 650, m 700, 750, 800, 850, 900, 950, 1000 L/m².

In embodiments, the secondary clarification is performed by a second filter, e.g. a depth filter, having a pore size that provides a retention range of at least about 0.2, 0.3. 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0 μM or more. In another embodiment the second filter has a pore size that provides a retention range of from about 0.4 μm to about 3.0 μm. In another embodiment the second filter has a pore size that provides a retention range of from about 0.4 μm to about 0.8 μm. In another embodiment the second filter has a pore size that provides a retention range of from about 1.0 μm to about 3.0 μm. In a particular embodiment the second clarification step is carried out by a second depth filtration.

In embodiments the primary clarification step and the secondary clarification step use a filter having the same pore size. In embodiments both of the first and second filters have a pore size that provides a retention range of from about 0.4 μm to about 0.8 μm. both of the first and second filters have a pore size that provides a retention range of from about 1.0 μm to about 3.0 μm.

In one embodiment, the secondary clarification step has a maximum throughput at least about 50, 60, 70, 80, 90, 100, 150, 200, 250, 300, 350, 400, 450, 500, 550, 600, 650, m 700, 750, 800, 850, 900, 950, 1000 L/m².

In one embodiment of the present invention, the primary clarification is preceded by a cell culture pretreatment step to enhance clarification efficiency such as flocculation.

As used herein the term “flocculation” refers to the aggregation, precipitation and/or agglomeration of insoluble particles, such as cell material including cells, cell debris, host cell proteins, DNA and other components, caused by the addition of a suitable flocculating agent to a fermented cell culture. By increasing the particle size of the insoluble components present in the fermented cell culture, the efficiency of separations, such as by clarification by filtration, is improved. Flocculation can be initiated by methods known in the art, including the reduction of the cell culture pH or the addition of a flocculating agent. Non limiting examples of flocculating agents include: calcium phosphate, caprylic acid, divalent cations or positively charged polymers like polyamine, polyethyleneimine (PEI), chitosan or polydiallyldimethylammonium chloride (e.g. pDADMAC), which induce the particles aggregation due to their interaction with the negatively charged surface of cells and cell debris. In one embodiment the flocculating agent is a positively charged polymer, preferably polyethyleneimine (PEI). In embodiments the flocculating agent is PEI and is added to the fermented cell culture at a concentration of from about 0.10% (v/v) to about 0.50% (v/v). In embodiments the flocculating agent is PEI and is added to the fermented cell culture at a concentration of from about 0.15% (v/v) to about 0.25% (v/v). In embodiments the flocculating agent is added to the fermented cell culture at a concentration of about 0.10% (v/v), 0.15% (v/v), 0.20% (v/v), 0.25% (v/v) or 0.30% (v/v). In embodiments the flocculating agent is PEI and added to the fermented cell culture at a concentration of about 0.15% (v/v) or about 0.25% (v/v).

In one embodiment the flocculating agent is directly added to the fermented cell culture fluid at the end of fermentation, and the flocculated material including the cells is removed from the cell culture fluid by filtration. In an embodiment, the flocculating agent is added prior to harvest.

In preferred embodiments the fermentation and clarification are performed as a closed method. As used herein the term “closed method” refers to a method or process that is performed in a closed sterile systems under controlled environment conditions without any risk of external contamination from the operator or laboratory environment. The use of a closed system is particularly important when developing GMP grade products. As such, in preferred embodiments that methods of the invention are closed methods that do not comprise centrifugation, as centrifugation cannot be performed in a closed system and requires transfer of the intermediate product (lysed cells) into centrifugation vials. The use of post-fermentation clarification can be performed by passing the fermented culture from the bioreactor as a flow in a closed tubing systems to the clarification filters, without any contact with the operator.

### Downstream Processing of the Clarified Protein

In embodiments the recombinant protein may be recovered directly from the clarified culture medium. Recovery of the recombinant protein may be followed by purification to ensure adequate purity of the recombinant protein. A variety of protein purification techniques are well-known to one of ordinary skill in the art. Suitable techniques for purification include, but are not limited, ammonium sulphate or ethanol precipitation, acid extraction, preparative gel electrophoresis, immunoadsorption, anion or cation exchange chromatography, phosphocellulose chromatography, hydrophobic interaction chromatography, affinity chromatography, immunoaffinity chromatography, size exclusion chromatography, liquid chromatography (LC), high performance LC (HPLC), fast performance LC (FPLC), hydroxyapatite chromatography, lectin chromatography, and immobilized metal affinity chromatography (IMAC). Most preferably, the recombinant proteins are purified by a combination of liquid chromatographic techniques including ion exchange, affinity and size exclusion.

The methods of the invention thus provide for substantially pure recombinant proteins. Substantially pure as used herein refers to a preparation or sample which is substantially free of contaminating components, proteins etc.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

### Example 1—Expressing Recombinant Vitronectin

### Engineering of Vitronectin Gene (Further—VTN)

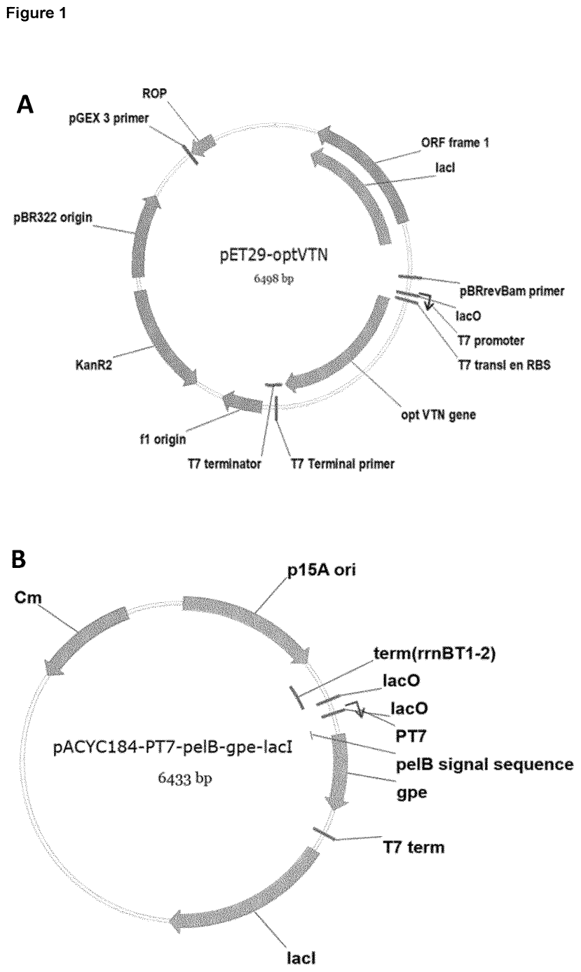

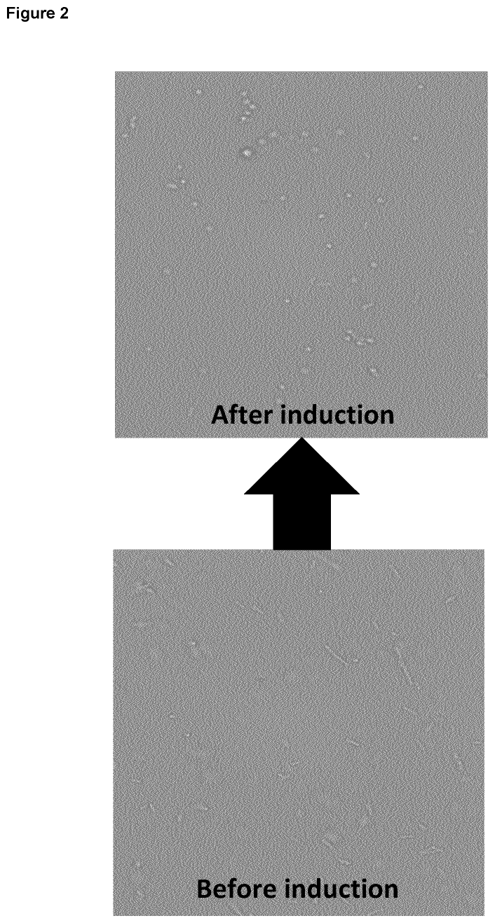

The target vitronectin gene (sequence disclosed herein as SEQ ID NO:2) was cloned into a pET vector following an inducible promoter PT7 (FIG. 1A). NCBI sequence ID: AAH05046.1, protein name: Vitronectin, origin of target gene: Homo sapiens.

### Engineering of T4 Bacteriophage Lysozyme Gene, Gpe.

T4 bacteriophage lysozyme gene gpe was cloned in to tight controlled pLATE11 vector (Cat. K1241, Thermo Fisher) to reduce the expression of lysis genes in uninduced cells. In additional, the signal peptide of pelB to translocate the produced protein to the periplasmic space was fused at the N-terminus of Gpe. The fragment containing the transcription terminator rrnBT1-T2 prevented basal gene expression from vector derived promoter-like elements, the T7 RNA polymerase promoter, which drives transcription of the cloned gpe gene, and two flanking lac operator sequences ensure tight control of gene expression, the Ptet promoter reduces basal expression from the T7 promoter, the T7 terminator which terminates transcription from the T7 promoter and the lac repressor which ensures tight control of basal expression from the T7 promoter was subcloned into the pACYC184 vector that has a p15A origin of replication and is compatible with plasmids harboring ColE1 ori. Recombinant plasmid was called pACYC184-PT7-pelB-gpe-lacl (FIG. 1B).

In this example, engineered polypeptide referred to as Gpe was employed. The sequence originated from lysozyme of Escherichia coli T4 bacteriophage and is disclosed herein as SEQ ID NO:1—NCBI Reference Sequence: NP_049736.1, protein name: glycoside hydrolase family protein (Escherichia phage T4). PelB signal sequence: 1-22, T4 bacteriophage lysozyme 23-164.

### Expression Strain Preparation

One Shot™ BL21 (DE3) E. coli cells were transformed with the plasmids pET29-optVTN and pACYC184-PT7-pelB-gpe-lacl plasmids and were plated on LB animal origin free (AOF) agar with kanamycin (50 g/l) and 25 μg/ml chloramphenicol. The transformation was based on the temperature shock method. Research cell bank (RCB) was produced by cultivating the transformants in liquid TB4 AOF medium with the required antibiotics at 37° C. and 220 rpm. A 50% sterile glycerol solution was used to produce 25% glycerol RCB which were aliquoted in cryovials and stored at −70° C.

### Cultivation Media

Transformations and plasmid propagations were performed on solid and liquid LB animal origin free (AOF) medium containing vegetable peptone (10 g/L), yeast extract (5 g/i), NaCl (10 g/l), and for solid medium 15 g/l microbiological agar, as well as the required antibiotics. Fed-batch and batch cultivations were performed in glycerol-based AOF terrific broth (TB4 AOF and TB AOF) with the following composition (per litre): Vegetable peptone 12 g, yeast extract 24 g, (NH₄)₂SO₄ 2.68 g, KH₂PO₄ 2.3 g, K₂HPO₄ 12.5 g and glycerol 4 to 10 g. Additionally, before cultivation the TB medium was supplemented with the following sterile solutions: 2 mL of (1M) MgSO₄ and 2 mL of trace element solution with the following composition (per litre): CaCl₂×2H₂O 0.5 g, ZnSO₄×7H₂O 0.18 g, MnSO₄×H₂O 0.1 g, Na₂-EDTA 20.1 g, FeCl₃×6H₂O 16.7 g, CuSO₄×5H₂O 0.16 g, CoCl₂×6H₂O 0.18 g; as well as 100 μL L-1 of thiamine hydrochloride (1 M), 1 mL 5 of kanamycin (50 mg mL/1) and 1 mL of chloramphenicol (30 mg/l).

### Expression and Autolysis Conditions in Shake Flasks

To select the optimal E. coli strain for VTN expression, E. coli JS007 and Invitrogen One Shot™ BL21 (DE3) Chemically Competent E. coli cells were transformed with both target recombinant plasmids pET29-optVTN and pACYC184-PT7-pelB-gpe-lacl. The level of protein expression in both strains was similar (FIG. 3B), however BL21 (DE3) cells showed a higher biomass yield (FIG. 3A) and BL21 (DE3) strain was therefore selected for further use in a cell bank development. Cells and media fractions were separated by centrifugation for 30 min, 14000 rpm, 4° C. Media fractions were stored at 4° C. Cell samples harvested from flask cultivations were resuspended in lysis buffer at the following ratio: 1 g of biomass were resuspended in 10 mL of lysis buffer (50 mM Tris-HCl, 50 mM NaCl, 0.1 mM EDTA, 0.1 mg/ml lysozyme). The biomass was sonicated for 60 seconds at 4° C. Soluble and insoluble protein fractions were separated by centrifugation for 20 min, 14000 rpm, 4° C. The total protein fraction represents cellular debris suspension (crude extract). Cell samples for SDS-PAGE separation were prepared as follows: 20 μL of crude extract sample, 25 μL of 4×SDS-PAGE loading buffer, 5 μL of 2M DTT and 50 μL of deionized water to obtain a final sample volume of 100 μL. Media samples for SDS-PAGE separation were prepared as follows: 70 μL of culture media, 25 μL of 4×SDS-PAGE loading buffer, 5 μL of 2M to obtain a final sample volume of 100 μL. Samples were heated for 10 min at 95° C. 10 μL of sample was applied to each lane of a gel.

### Expression and Autolysis Conditions in Bioreactor

Cultivation media and feed solution—preinoculum and inoculum propagations were performed in glycerol-based animal origin free (AOF) terrific broth (TB4 AOF) with the following composition (per litre): Vegetable peptone 12 g, Yeast extract 24 g, (NH4)₂SO4 2.68 g, KH₂PO₄ 2.3 g, K₂HPO₄ 12.5 g and vegetable glycerol 4 g. Additionally, before seeding the TB4 medium was supplemented with the following sterile solutions: 3 mL/L of (1M) MgSO₄ and 3 mL/L of trace element solution (TES) with the following composition (per litre): CaCl₂×2H₂O 0.5 g, ZnSO₄×7H₂O 0.18 g, MnSO₄×H₂O 0.1 g, Na₂-EDTA 20.1 g, FeCl₃×6H₂O 16.7 g, CuSO₄×5H₂O 0.16 g, CoCl₂×6H₂O 0.18 g; as well as 3 mL/L of thiamine hydrochloride (10 g/L), 1 mL/L of kanamycin (50 mg mL/1) and 1 mL/L of chloramphenicol (25 mg/l). Terrific broth medium with 15 g glycerol (TB15 AOF).

### Preinoculum Preparation

A single colony from E. coli BL21(DE3) [pET29-optVTN and pACYC184 PT7-PeB-gpe-lacl] transformants plate was transferred to 250 mL shake flasks with 50 ml of TB4 AOF medium with supplements and antibiotics and was incubated for 8±h at 37° C./230 rpm. A culture optical density (OD600) was measured at the end of cultivation and seed volume for inoculum flask was calculated by formula: 4/ODpreinoculum=mL.

### Inoculum Preparation.

The calculated preinoculum volume was transferred in to 2000 mL shake flaks with 500 ml of TB4 AOF medium with supplements and antibiotics and was incubated for 17±1 h at 25° C./230 rpm. A culture optical density (OD600) is measured at the end of cultivation. Seed volume for bioreactors is 2% of volume of total media.

### Bioreactor Processes

Batch phase was performed in a 300 L S.U. fermenter (250 L media). Culture growth dynamic was monitored by optical density measurements at 600 nm wavelength. The samples from bioreactors were taken at different process stages and analyzed. The bioreactors seeded with overnight inoculum (2% by volume). The initial culture parameters before target protein and simultaneous lysis induction were as follows: the dissolved oxygen (DO) control was maintained at 30% by automatic cascade: air flow (from 75 to 250 L/min); the stirrer rate (from 100 to 375 rpm); pure oxygen flow (from 0 to 160 L/min). Culture pH was controlled at 7.0±0.05 by automatic addition of NH₄OH (25%) or H3PO4 (42.5%). The growth temperature before VTN induction (VTN induction point: 3.5-5.5 OD) was maintained at 37° C. Before induction with 0.1 mM IPTG the temperature in all processes was reduced from 37 to 25° C. IPTG addition simultaneously induce target protein expression and lysozyme expression inside the cells. After 0.5 hours after induction DO was set to 3%. After 2-4 hours after induction add 18750000 u of benzonase (universal nuclease), then changed stirrer to min 100 rpm, min air flow to 40 L/min. Continued fermentation 17-22 h after induction. At the end of the fermentation process 18750000 u of benzonase (universal nuclease) was added and stirring continued for 15 min.

### Analysis of Autolysis and Expression of VTN Protein.

Cells and media fractions were separated by centrifugation for 30 min, 14000 rpm, 4° C. Media fractions were stored at 4° C. Cell samples harvested from flask or fermenter cultivations were analyzed by SDS-PAGE electrophoresis. Cell samples were collected at different cultivation times (before induction and after induction). Soluble and insoluble protein fractions were separated by centrifugation for 20 min, 14000 rpm, 4° C. The soluble protein fraction or supernatant after centrifugation represented effectiveness of the autolysis process and what part of the proteins were soluble. Insoluble fraction represented undisrupted cells and insoluble proteins. Cell samples for SDS-PAGE separation were prepared as follows: 20 μL of crude extract sample, 25 μL of 4×SDS-PAGE loading buffer, 5 μL of 2M DTT and 50 μL of deionized water to obtain a final sample volume of 100 μL. Media samples for SDS-PAGE separation were prepared as follows: 70 μL of culture media, 25 μL of 4×SDS-PAGE loading buffer, 5 μL of 2M to obtain a final sample volume of 100 μL. Samples were heated for 10 min at 95° C. 10 μL of sample was applied to each lane of a 4-12% gradient SDS-PAGE gel.

### Clarification

It was determined that P100 and KS50P media with a pore size of 1.0-3.0 μm, removed cells and cell debris from different optical density. Vitronectin fermentation culture effectively and generated clear, clarified supernatant. Experiments were repeated with vitronectin fermentation culture grown at a manufacturing scale (150 L) and it was calculated that with a 0.05 m² size P100 and KS50P media filter, 0.6-0.7 L fermentation culture (OD600 nm=15) could be filtrated. For an experimental batch, development results were extrapolated, and the experiment was performed with 150 L fermentation culture (OD600 nm=17) and 7×2 m² Stax™ 1.0-3.0 μm, P100 media, depth filter capsules. Since membrane clogging was not observed and clear filtrated supernatant was obtained, these conditions could be maintained for clarification at a manufacturing scale.

For removal of the remaining cell debris and supernatant preparation for subsequent stages, clarified cell culture was filtrated through 0.2 μm filter. A 1 m² membrane area size Supor® EX Grade ECV filter was tested and all of the 150 L clarified solution was filtered successfully.

For downstream applications buffer solution in clarified media was exchanged by ultrafiltration. Ultrafiltration was performed by tangential flow filtration system (TFF) using TFF filter with a pore size of 30 kDa filter. The filtration area was 0.01 m² for a 1 L media solution.

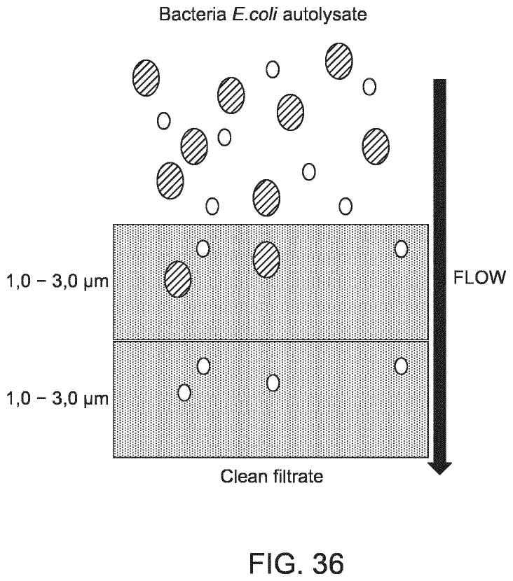

In more detail, the E. coli autolysate clarification process involved the following steps (FIG. 36).

Starting material: 250 L of cell lysed cell culture, after 16-18 h of target protein expression in the stirred tank reactors. Final OD of the cells/cell debris ~15-30 OD. Analysis showed that induced cell lysis resulted in 75-80% cell lysis efficiency from total cell amount.

During all manipulations within stirred tack bioreactor (Hyperforma™ 300 L, Thermofisher Scientific) all automatic controls in fermentation vessel: Air/O2 flow, pH was in “off mode”.The stirring was maintained at 375 rpm and the temperature was set to 23° C.

Removal of nucleic acids and clarification was carried out as follows:

Nucleic acid hydrolysis: Universal nuclease is added to bioreactor to achieve final concentration of 250 U/ml and incubated for 30-60 min at 23° C.

Filter preparation: STAX filter capsule is washed with 10 filter volumes of water without exceeding back pressure of 0.2 BAR.

The lysed cell suspension was clarified by filtering through the depth filtration capsules(1-3 μM (K100P) (Pall, ID 7007836) (about 0.05 m² of filter area is needed to filtrate 1 L of fermentation media) and subsequently is filtered through 0.22 μm filter capsule (Pall, ID NP6UEAVP1S) without exceeding back pressure of 1.2 BAR. Filtration alternatives: K050P (Pall, ID 7007786) Retention 0.4-0.8 μM.

### Results and Discussion

E. coli is not a perfect host especially for closed single use fermentation because it normally does not secrete proteins into the extracellular medium. The cell disruption step becomes a limitation and not cost effective at the pilot or industrial scales when the goal is the implementation of closed and/or continues process flow. The present invention represents an approach for cell disruption that is a programmed cell lysis system based on the expression of a cloned T4 bacteriophage gene gpe to produce a lytic protein. Gene gpe encodes a lysozyme which degrades the peptidoglycan of the E. coli cell wall.

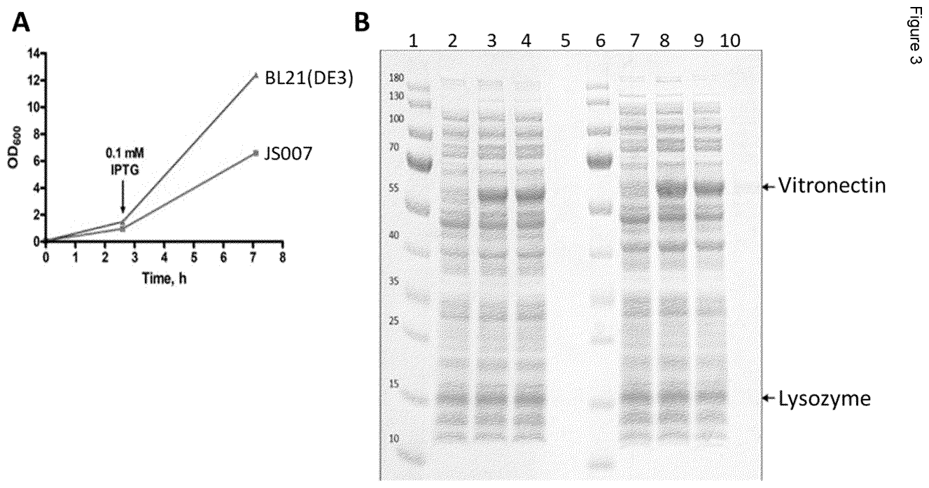

Self-disruptive Escherichia coli that produces foreign target protein was developed. E. coli was co-transformed with two vector plasmids a target gene expression vector E. coli BL21(DE3) [pET29-optVTN for protein VTN expression and lysis gene expression vector pACYC184-PT7-pelB-gpe-lacl (or pARApeIB-pge). The lytic protein was produced after the expression of the target gene, resulting in simplification of the cell disruption process. As disclosed herein, the expression of cloned T4 phage gene gpe was used for the disruption of E. coli that produced VTN as a target protein. The expression of gene gpe did not lead to prompt cell disruption but weakened the cell wall. The translocation of lytic protein Gpe from the cytoplasm to the periplasm facilitated enzymatic degradation of murein layer which is responsible for maintaining cell strength. Microscopic observation revealed that cells producing Gpe in the periplasmic space after 4 h went through a morphological change, from rod-shaped to elliptical spheroplasts (FIG. 2).

After a 4 hour induction 0.1 mM IPTG of E. coli BL21(DE3) [pET29-optVTN and pACYC184 PT7-PeB-gpe-lacl]VTN protein was released to the culture medium. Maximum VTN protein production (about 0.5 g/l) in shake flask was obtained after overnight expression of E. coli BL21(DE3) [pET29-optVTN and pACYC184 PT7-PeB-gpe-lacl] at 25° C. when lysozyme induction at 0.1 mM IPTG was performed together with target gene induction (induction point: 3.5-5.5 OD)—FIG. 3.

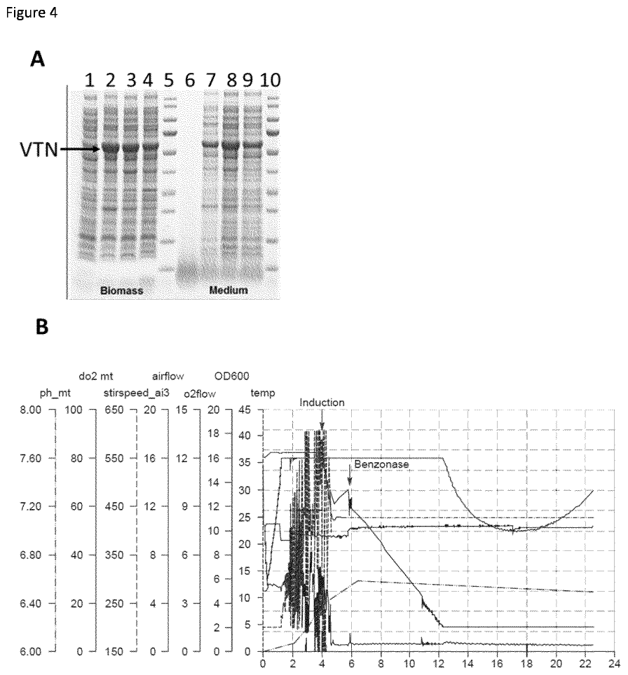

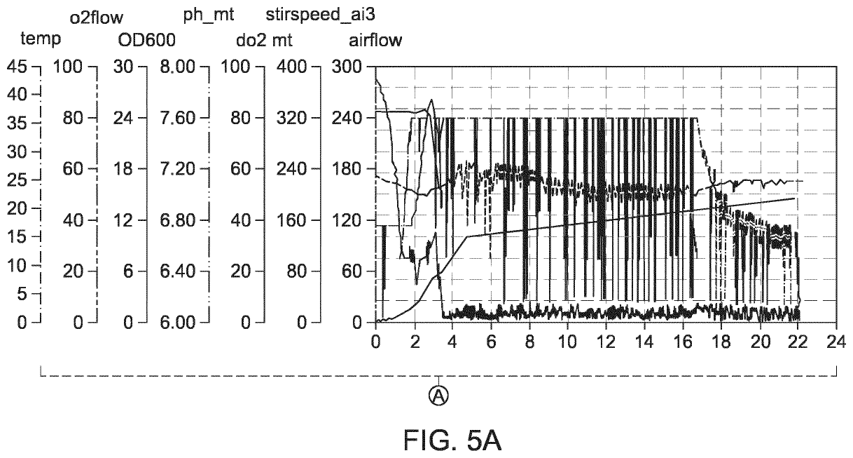

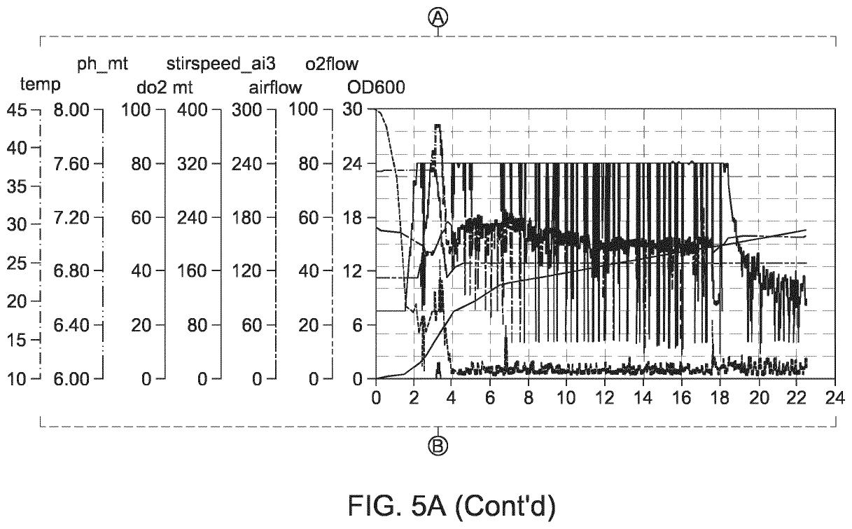

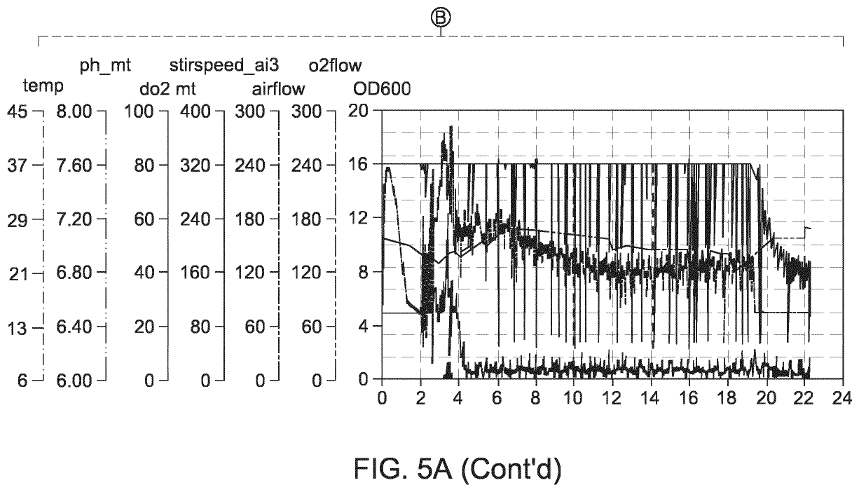

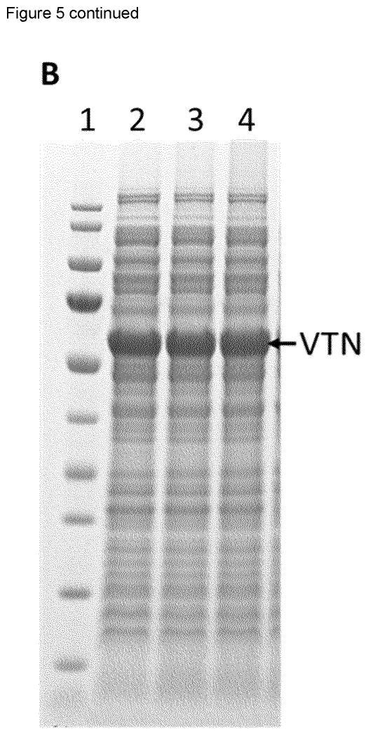

Optimized protein expression conditions were successfully transferred to a 3 L bioreactor (Biostat) and scaled up in 300 L SUF. After the optimization of the fermentation process the yield of VTN protein was about 0.1-0.2 g/l—FIG. 4 and FIG. 5.

Clarification process—the given clarification conditions ensured that all cell debris and insoluble proteins were removed from the solution leaving only the soluble fraction of proteins in which the target protein (VTN) concentration is about 0.1-0.2 g per 1 liter of the starting material.

Depth filtration-based clarification of cell debris after nuclease treatment—as a result very small particles of membrane segments were generated. This was demonstrated successfully using unusually large pores filters. E. coli cell size is: 1.0-2.0 micrometres long, with radius about 0.5 micrometres. Primary clarification approaches which are usually recommended for E. coli are tangential flow filtration clarification <0.2 micrometres pore size or centrifugation, which is the most common approach for clarification of cellular debris.

### Example 2—Expressing Recombinant Inorganic Pyrophosphatase (IPP)

### Engineering of IPP Gene

The target IPP gene was cloned into a pET21b vector under an inducible promoter PT7.

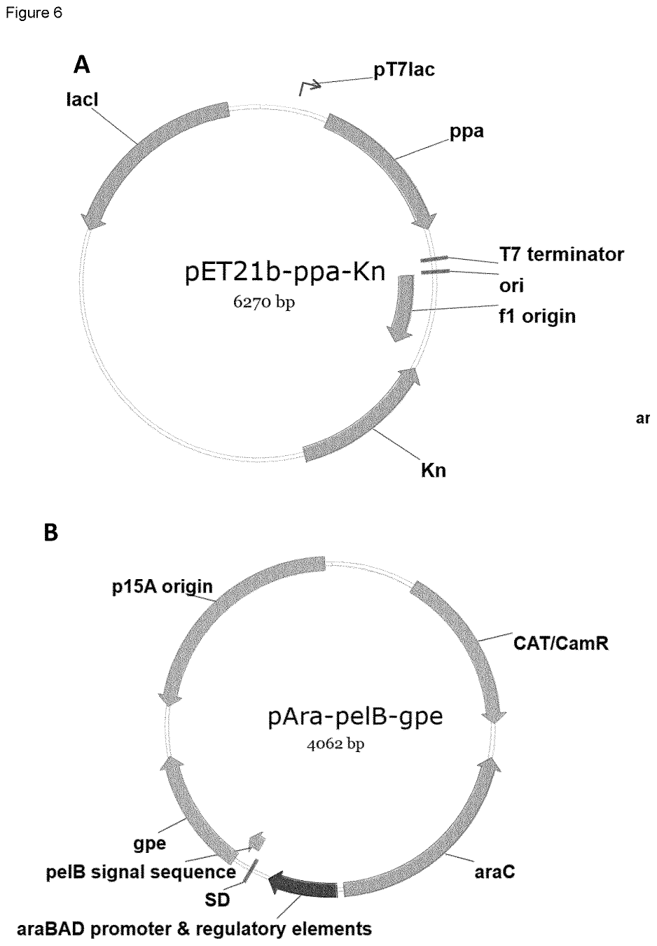

Using gene engineering the pET21b-ppa plasmid resistance to Ampicillin (Amp) was changed to Kanamycin (Km) resistance to arrive at the final plasmid, pET21b-ppa-Kn. The plasmid was sequenced by Sanger sequencing and approved as applicable for further investigation (FIG. 6A)—GeneBank: X13253.1, 99% identity, protein sequence: NCBI Reference Sequence: CAA31629.1, protein name: Ribonuclease Inhibitor. This sequence is disclosed herein as SEQ ID NO:3.

### Engineering of T4 Bacteriophage Lysozyme Gene, Gpe.

T4 bacteriophage lysozyme gene gpe with signal peptide of pelB was cloned into pAra vector (Thermo Fisher) under araB promoter (plasmid pAra-pelB-gpe). The plasmid pAra-pelB-gpe carries an origin of replication derived from pACYC and a chloramphenicol-resistance gene gene which allows the use of E. coli expression systems containing ColE1-type plasmids (FIG. 6B).

In this example, engineered polypeptide referred to as Gpe which sequence originated from lysozyme of Escherichia coli T4 bacteriophage was employed—NCBI Reference Sequence: NP_049736.1, protein name: glycoside hydrolase family protein [Escherichia phage T4]. PelB signal sequence: 1-22, T4 bacteriophage lysozyme 23-164. This sequence is disclosed herein as SEQ ID NO:1.

### Expression Strain Preparation

The E. coli strain BL21(DE3) (Thermo Fisher Scientific) was transformed with the plasmids pET21b-ppa-Kn and pAra-pelB-gpe (carrying the gene for the T4 bacteriophage lysozyme, gpe) and then plated on LB (animal origin free) AOF agar with kanamycin (50 g/I) and chloramphenicol (25 g/l). Transformants with both plasmids were plated on LB agar containing kanamycin (50 mg/l) and chloramphenicol (25 mg/l). The transformation was based on the calcium temperature shock method. A Research cell bank (RCB) was produced after 3 h of cultivation of the transformants in liquid TB4 AOF medium with the required antibiotics at 37° C. and 220 rpm. A 50% sterile glycerol solution was used to produce 25% glycerol RCB which were aliquoted in cryovials and stored at −70° C.

### Cultivation Media

The E. coli BL21(DE3) [pAra-pelB-gpe]/pET21b-ppa-Kn] transformants propagations were performed on solid LB AOF medium containing Vegetable peptone (10 g/L), Yeast extract (5 g/l), NaCl (10 g/l), and for solid medium 15 g/l microbiological agar, as well as the required antibiotics: 1 mL/L of kanamycin (50 mg mL/I) and 1 mL/L of chloramphenicol (25 mg/l). Preinoculum and inoculum preparation was performed in glycerol-based animal origin free (AOF)S-Me broth (S-Me AOF) with the following composition (per litre): Yeast extract 5 g, NH₄Cl₂ g, NaCl 0.5, KH₂PO₄ 3 g, K₂HPO₄ 6 g, citric acid 1.5 g and vegetable glycerol 10 g. Additionally, before seeding the S-Me AOF medium was supplemented with 1 mL/L of kanamycin (50 mg mL/L), 1 mL/L of chloramphenicol (25 mg/L), 2 mL/L of MgSO₄(1M), 1 mL/L Thiamine-HCl (10 mg/mL), 2 mL/L of trace element solution (TES) with the following composition (per litre): CaCl₂×2H₂O 0.5 g, ZnSO₄×7H₂O 0.18 g, MnSO₄×H₂O 0.1 g, Na₂-EDTA 20.1 g, FeCl₃×6H₂O 16.7 g, CuSO₄×5H₂O 0.16 g, CoCl₂×6H₂O 0.18 g. Benzo AOF medium (Benzo AOF) with the following composition (per litre): Yeast extract 24 g, (NH₄)₂SO₄ 2.68 g, NH₄Cl₂ 1.5 g, KH₂PO₄ 6 g, K₂HPO₄4 g, vegetable glycerol 10 g; supplemented with 2 mL/L of MgSO₄(1M), 1 mL/L Thiamine-HCl (10 mg/mL), 1 mL/L of kanamycin (50 mg mL/I) and 1 mL/L of chloramphenicol (25 mg/l), 2 mL/L of trace element solution (TES) was used as starting bioreactor media.

Batch mode cultivations and recombinant protein synthesis in shake flasks: the inoculums for batch protein production in the shake flasks were prepared by overnight cultivation of the selected clone in 2000 mL shake flasks with 500 ml of S-Me medium at 25° C. and 180 rpm. For protein production the corresponding inoculum culture (0.1 start OD600) was transferred to fresh Benzo AOF medium containing 10 g/l of vegetable glycerol to a final volume of 50 mL in 250 ml baffled Erlenmeyer shake flasks. Cultures were cultivated at 37° C. and 220 rpm until they reached a cell density of OD600 1.5-2.0. Induction was performed with 0.1 mM IPTG. The temperature was changed at the induction point to 30° C. and the culture was continued for 3 hours at 220 rpm. The recombinant protein induction was performed in two ways—with and without cell lysis induction. For lysis induction 1250 μl of 20% solution of L-arabinose was added to culture. Cultures with or without lysis induction were grown at 30° C., 220 rpm.

Expression and autolysis conditions in bioreactor: a small amount of E. coli BL21(DE3) [pAra-pelB-gpe]/pET21b-ppa-Kn]biomass from solid LB AOF plate or 1 mL of RCB was transferred in to 250 mL shake flaks with 50 ml of S-Me AOF medium with supplements and antibiotics. Flasks were incubated for 7.5±0.5 h at 37° C./180 rpm. A culture optical density (OD600) was measured at the end of cultivation and seed volume for inoculum flask was calculated by formula: 0.25/ODpreinoculum*1000=μL. The calculated preinoculum volume was transferred in to 2000 mL shake flaks with 500 ml of S-Me AOF medium with supplements and antibiotics. The flasks was incubated for 17±1 h at 25° C./180 rpm. A culture optical density (OD600) was measured at the end of cultivation. Inoculum seed volume for bioreactors was calculated by formula: V initial bioreactor media/50=V inoculum (2% of initial bioreactor media volume). Batch phase was performed in a 5 L Univessel Glass Biostat A bioreactors (Sartorius) with an Benzo AOF medium volume of 3 litres. Culture growth dynamic was monitored by optical density measurements at 600 nm wavelength. The samples from bioreactors were taken at different process stages and analyzed. The bioreactors seeded with overnight inoculum (2% of initial bioreactor media volume). The initial culture parameters before target protein and simultaneous lysis induction were as follows: the dissolved oxygen (DO) control was maintained at 30% by automatic cascade: air flow (from 1.6 to 4.8 Lpm); the stirrer rate (from 420 to 800 rpm); pure oxygen flow (from 0 to 4.8 Lpm). Culture pH was controlled at 7.0±0.1 by automatic addition of NH40H (25%) or H3PO4 (42.5%). The growth temperature before IPP induction (IPP induction point: 2.5±0.5 OD) was maintained at 37° C. After induction with 0.1 mM IPTG the temperature in all processes was reduced from 37 to 30° C. 0.5 h after IPTG induction DO profile was started: 30% to 3% over 1h. 2-4 h after IPTG induction minimum stirrer speed was changed to 200 rpm. The recombinant protein synthesis was performed in two ways—with and without cell lysis induction. For the cell lysis arabinose was used to induce lysozyme expression inside the cells. An induction phase length: 22±2 h. After induction phase gas supply and pH control were stopped, harvesting/clarification steps were initiated.

### Clarification of the Fermentation Culture

The culture clarification was performed using P250 and P050 depth filtration capsules. Culture supernatant was transferred from bioreactor straight on the P250 equilibrated with buffer IPP-A1 (20 mM Tris-CI pH=8, 1 mM EDTA, 35 mM AmS). All 100 L of culture was filtered through 2 m² capsule with pressure <0.5 bar. OD600 of the filtrate dropped from 27 to 14. Then the filtrate was transferred on P050 capsule. In total around 50 L of culture filtered through 2 m² capsule before clogged (reached 2 bar). Subsequently, the filtrate filtered through 0.22 um filter. The final filtrate concentrated on 0.11 m² Pall membrane (10 kDa) from 10 L to 1 L and buffer exchanged 8× to IPP-A1.

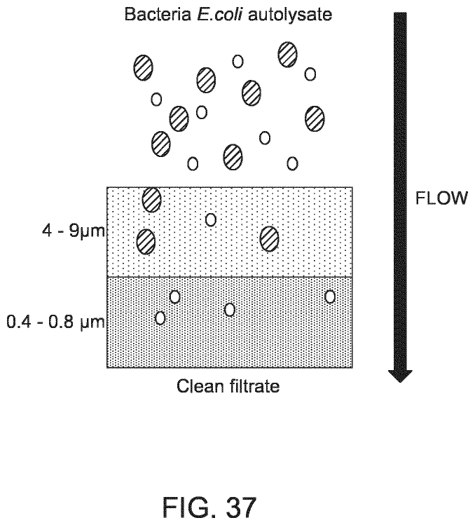

In more detail, the clarification steps involved (FIG. 37):

Starting material: 250 L of cell lysed cell culture, after 16-18 h of target protein expression in the stirred tank reactors. Final OD of the cells/cell debris ~15-30 OD. Analysis showed that induced cell lysis resulted in 75-80% cell lysis efficiency from total cell amount.

During all manipulations within stirred tack bioreactor (Hyperforma™ 300 L, Thermofisher Scientific) all automatic controls in fermentation vessel: Air/O2 flow, pH was in “off mode”. The stirring was maintained at 375 rpm and the temperature was set to 23° C.

Clarification: Filter preparation. Required amount of STAX filter capsules K050P (Pall, ID 7007836) were washed/equilibrated with 2 filter volumes of buffer:20 mM Tris-CI pH=8, 1 mM EDTA, 35 mM AmSO4, without exceeding the back pressure of 0.2 BAR.

Primary Clarification: the lysed culture was clarified by filtering through the depth filtration capsules P250 (Pall, ID SXLP250402SP). Retention 4-9 μM (about 0.02 m² of filter area is needed to filtrate 1 L of fermentation media). OD600 of the filtrate dropped from 27 to 14. Pressure up to 0.5BAR.

Secondary clarification: The filtrate was loaded on P050 STAX capsules (Pall, ID 7007786) (Retention 0.4-0.8 μM). (about 0.05 m² of filter area is needed to filtrate 1 L of media). and subsequently is filtered through 0.22 μm filter capsule (Pall, ID NP6UEAVP1S) without exceeding back pressure of 2.0 BAR. Filtration alternatives: 1-3 μM (P100) (Pall, ID 7007836).

### Results and Discussion

IPP Protein Expression with Lysis Induction

E. coli is not a perfect host especially for closed single use fermentation process because it normally does not secrete proteins into the extracellular medium. The cell disruption step becomes a limitation and is not cost effective at the pilot or industrial scale when the goal is the implementation of closed and/or continues process flow. The present approach for cell disruption is the programmed cell lysis system based on the expression of a cloned T4 bacteriophage gene gpe to produce a lytic protein. Gene gpe encodes a lysozyme which degrades the peptidoglycan of E. coli cell wall.

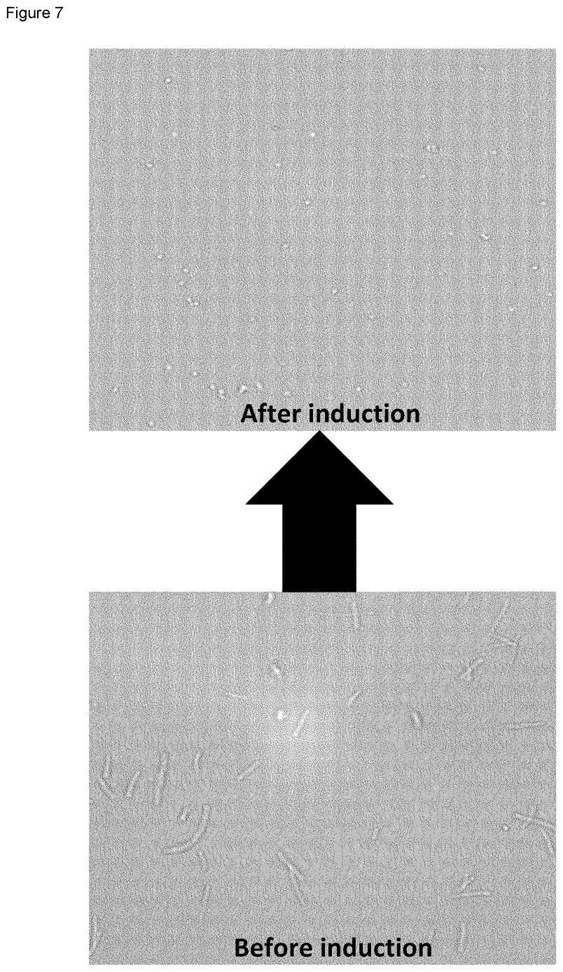

Self-disruptive Escherichia coli that produces foreign target protein was developed. E. coli BL21(DE3) was co-transformed with two vector plasmids: a target gene expression vector pET21b-ppa-Kn and a lysis gene expression vector pAra-pelB-gpe. The lytic protein was produced after the expression of the target gene, by adding arabinose to final concentration 0.05% resulting in simplification of the cell disruption process. In this example, the expression of cloned T4 phage gene gpe was used for the disruption of E. coli that produced IPP as a target protein. The expression of gene gpe did not lead to prompt cell disruption but weakened the cell wall. The translocation of lytic protein Gpe from the cytoplasm to the periplasm facilitated enzymatic degradation of murein layer which is responsible for maintaining cell strength. Microscopic observation revealed that cells producing Gpe in the periplasmic space after 4 h went through a morphological change, from rod-shaped to elliptical spheroplasts (FIG. 7).

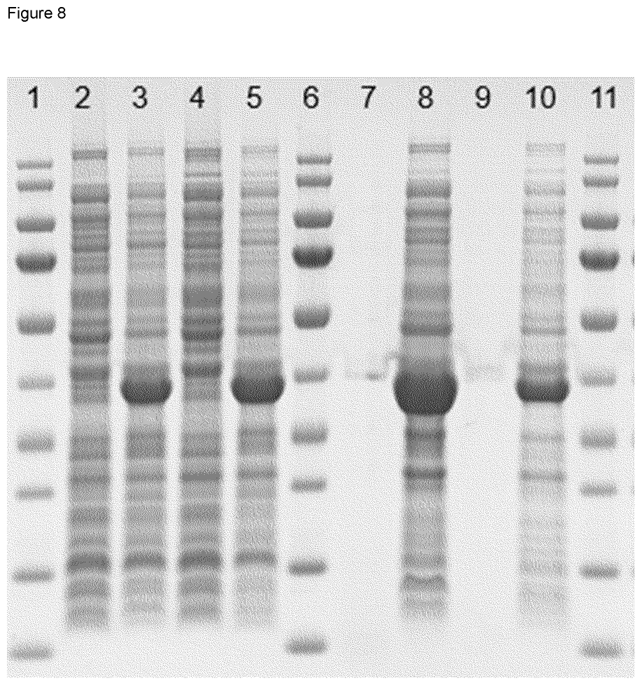

For testing IPP induction in BL21(DE3) [pAra-pelB-gpe]/[pET21b-ppa-Kn] with lysis, experiments in flasks and bioreactors were performed and media samples after 20-24 hours of induction were taken. SDS PAGE analysis of media samples shown that Inorganic pyrophosphatase was released to the culture medium in both—flask and bioreactor (FIG. 8, sample 10 and sample 8 respectively). Debris of E. coli cells were observed in the culture medium, indicating that cell lysis had occurred. Maximum Inorganic pyrophosphatase production in shake flask was obtained after overnight expression when lysozyme induction with arabinose was performed 3 hours after target gene induction. The protein band in SDS-PAGE gel (FIG. 8, 10 sample) clearly indicated presence of Inorganic pyrophosphatase protein (~32 kDa) in growing media (indicated by arrow). It was noted that a similar amount of IPP after protein synthesis induction in flask was detected in the biomass (sample 5).

The IPP expression process in Biostat A stirred bioreactor was designed with the initial medium volume of 3 L and after fermentation parameters optimization target protein yield in culture supernatant was increased compared to flask experiments (FIG. 8, sample 8).

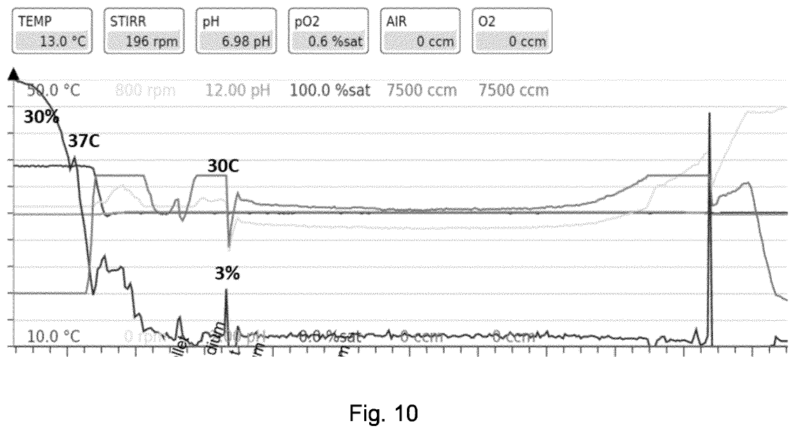

As shown in FIG. 10, the temperature of growth before IPP induction (induction point: 2.5±0.5 OD) was maintained at 37° C.

After induction with 0.1 mM IPTG the temperature in all processes was reduced from 37 to 30° C. over 0.5 h time span. DO profile was set to 3%. Lysozyme induction was initiated by addition of 0.5 g/L arabinose. 2 h after IPTG induction minimum stirrer speed was changed to 200 rpm. After induction phase (20h) measured culture reached 17 OD.

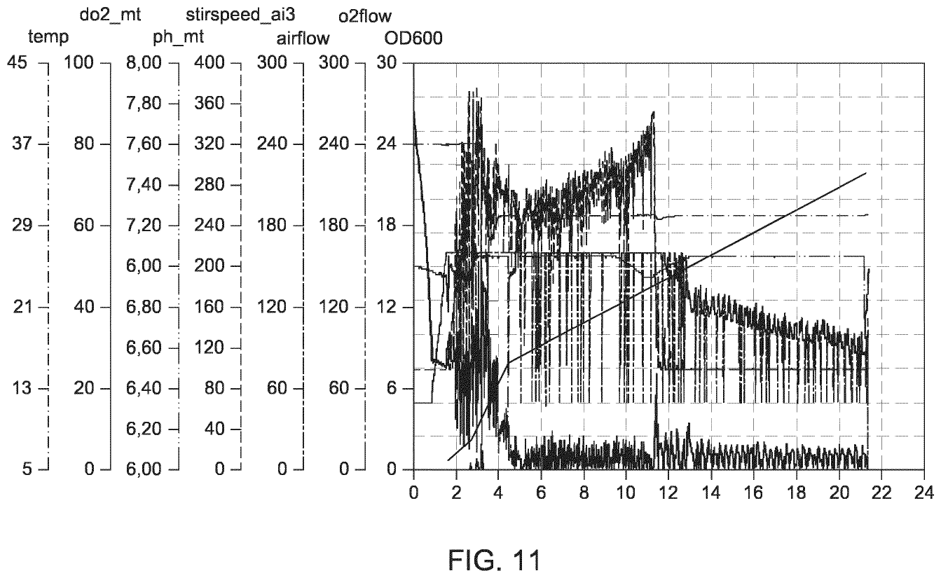

Biosynthesis parameters that were used in development were upscaled from 51 Univessel Glass Biostat A bioreactors to 300 L single use bioreactor capabilities in order to ensure that optimized process would be transferrable to manufacturing facilities. Large scale fermentation (FIG. 11) was performed in 300 L SUF bioreactor using Benzo-AOF1 medium at 100 L starting volume.

Protein expression induced with 0.1 mM IPTG at OD600=2.4 o. u. Protein is visible in a medium after 23.5 h fermentation. Yield of the protein and protein impurities profile is similar comparing with low scale (3 L) fermentation in R&D facilities (laboratory notebook D. Kavaliauskas 2019/01). After the optimization of fermentation process the yield of IPP protein was 1.1 g/L. The clarification steps were performed as described in the materials and methods above. Since membrane clogging was not observed and clear filtrated supernatant was obtained, these conditions were maintained for manufacturing scale clarification. The given clarification conditions ensured effective cell debris removal. After optimized clarification process media was prepared for downstream applications and contains only soluble proteins.

PPI Protein Expression with Basal Gpe Expression without Targeted Lysis Induction

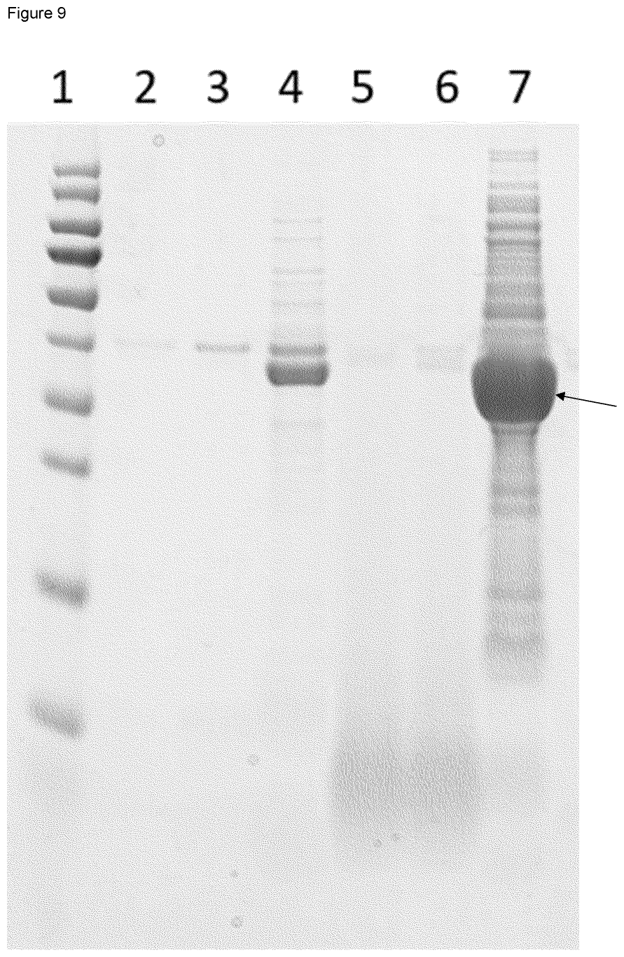

Protein synthesis induction experiments in the flasks and bioreactors were performed without targeted lysis induction by using basal leakage expression feature of T4 lysozyme. Initially IPP protein level was determined in media by growing culture with target protein—IPP synthesis induction, but without targeted induction of lysis system. The media samples for testing of target protein amount from flasks and bioreactor were taken after 3 and 20 hours after induction and analyzed by SDS-PAGE (FIG. 9). IPP protein was detected in both (flask and bioreactor) media samples even without lysis induction. The highest amount of target protein in media samples from bioreactor after 20 hours (FIG. 9, 7 sample, shown by arrow). The protein in media could be released possibly due to basic level of lysozyme expression from pAra-pelB-gpe plasmid, which presence in expression strain.

Depth filtration-based clarification of cell debris without nuclease treatment and without addition of any agents was demonstrated successfully using unusually large pores of 4-9 um filters, which is unusually large for cell debris. E. coli cell size is: 1.0-2.0 micrometres long, with radius about 0.5 micrometres. Primary clarification approaches which are recommended for E. coli are tangential flow filtration clarification <0.2 micrometres pore size or centrifugation which is the most common approach for clarification of cellular debris, large or small flocculants.

### Example 3—Expressing Recombinant Moloney Murine Leukemia Virus (MuLV) Reverse Transcriptase Gene (RT)

### Engineering of Moloney Murine Leukemia Virus MuLV RT

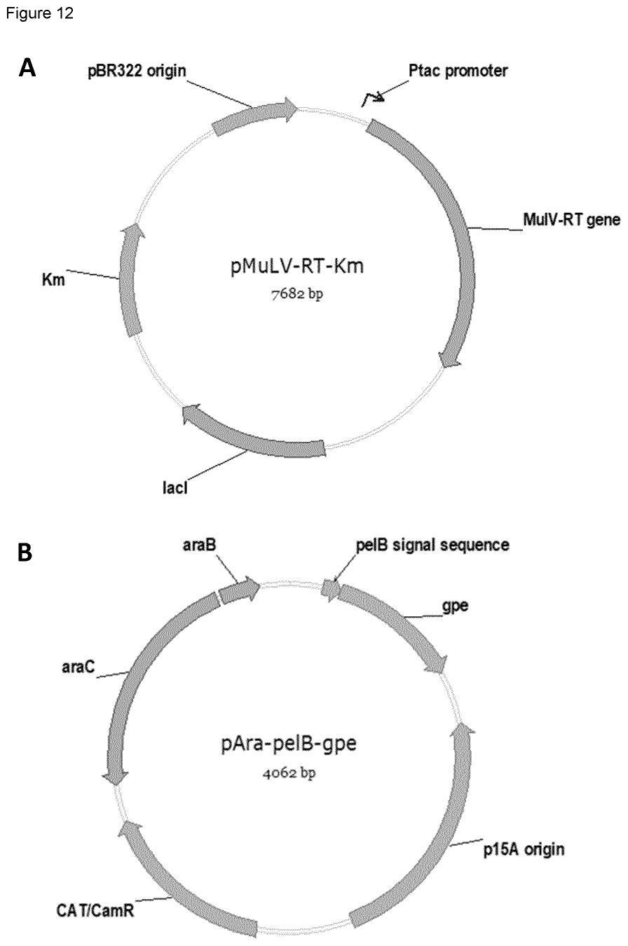

Gene of MuLV-RT was cloned under control of an inducible promoter Ptac. The recombinant plasmid was called pMuLV-RT-Km (FIG. 12A). The amino acid sequence of MuLV-RT is disclosed herein as SEQ ID NO:4.

### Engineering of T4 Bacteriophage Lysozyme Gene Gpe

T4 bacteriophage lysozyme gene gpe with signal peptide of pelB was cloned in to pAra vector (Thermo Fisher) under araB promoter (plasmid pAra-pelB-gpe). The plasmid pAra-pelB-gpe carries an origin of replication derived from pACYC and a chloramphenicol-resistance gene gene which allows the use of E. coli expression systems containing ColE1-type plasmids (FIG. 12B).

For this Example, engineered polypeptide referred as Gpe which sequence originated from lysozyme of Escherichia coli T4 bacteriophage was used. NCBI Reference Sequence: NP_049736.1, protein name: glycoside hydrolase family protein [Escherichia phage T4]. PelB signal sequence: 1-22, T4 bacteriophage lysozyme 23-164. The amino acid sequence of Gpe with PelB signal sequence is disclosed herein as SEQ ID NO:1.

### Expression Strain Preparation

The E. coli strain JS007 (Thermo Fisher Scientific) was transformed with the plasmids pMuLV-RT-Km (carrying the gene for reverse transcriptase expression) and pAra-pelB-gpe (carrying the gene for the T4 bacteriophage lysozyme gpe) and plated on LB agar with kanamycin (50 g/l), chloramphenicol (25 g/l). The transformation was based on the calcium temperature shock method. A Research cell bank was produced.

### Cultivation Media

Transformations and plasmid propagations were performed on solid and liquid LB medium containing peptone (10 g/L), Yeast extract (5 g/l), NaCl (10 g/l), and for solid medium 15 g/l microbiological agar, as well as the required antibiotics. Fed-batch and batch cultivations were performed in glycerol-based microbiological broth with the following composition (per litre): Peptone 12 g, Yeast extract 5 g, (NH₄)₂SO₄ 2.68 g, NaCl 1.5 g, KH₂PO₄ 6 g, K₂HPO₄ 4 g and glycerol 4 to 10 g. Additionally, before cultivation the TB10 medium was supplemented with the following sterile solutions: 2 mL of (1M) MgSO₄ and 2 mL of trace element solution with the following composition (per litre): CaCl₂×2H₂O 0.5 g, ZnSO₄×7H₂O 0.18 g, MnSO₄×H₂O 0.1 g, Na₂-EDTA 20.1 g, FeCl₃×6H₂O 16.7 g, CuSO₄×5H₂O 0.16 g, CoCl₂×6H₂O 0.18 g; as well as 1 mL of kanamycin (50 mg mL/1) and 1 mL of chloramphenicol (30 mg/l).

### Expression and Autolysis Conditions in Shake Flask

The inoculum for batch protein production in the shake flask was prepared by overnight cultivation in 2000 mL shake flaks with 500 ml of TB4 medium containing 4 g/l of glycerol at 37° C. For protein production the corresponding inoculum culture was transferred to fresh terrific medium containing 10 g/l of glycerol to a final volume of 50 mL in 250 ml baffled Erlenmeyer shake flasks. Cultures were cultivated at 37° C. and 220 rpm until they reached a cell density of OD600 1.5. For expression of the target gene in shake flask, isopropyl-β-D-galactoside (IPTG) was added to give a final concentration of 0.1 mM. After 5 hours for expression of the lysis gene in shake flask, L-arabinose was added to give a final concentration of 0.5 g/l. The temperature was changed at the induction point to 25° C. After the addition of L-arabinose, samples were removed after 1 hours. The optical density of the medium at 600 nm (OD600) was measured to estimate the cell concentration.

### Sample Preparation for SDS-PAGE

1 ml culture samples were harvested from flasks, 1 Pierce™ Universal Nuclease for Cell Lysis is added to 1 ml cell culture and centrifuged for 1 min at 13000 rpm. The media fraction was then used for further analysis—40 μl of media fraction had 50 μl of 2× Tris-Glycine SDS sample buffer added, followed by 10 μl of 10× Sample Reducing Agent to obtain a final sample volume of 100 μl. Cell pellet was resuspended in to 100 μl of Tris-Glycine SDS sample buffer with Reducing Agent. Samples were heated for 10 min at 95° C. 5 μL of sample was applied to each lane of a 12% SDS-PAGE gel.

### Expression and Autolysis Conditions in Bioreactor

The inoculum for batch protein production in the shake flask was prepared by overnight cultivation of transformant in 2000 mL shake flaks with 500 ml of BenzoAOF10 medium containing 10 g/l of glycerol at 25° C. 180 rpm. Batch phase was performed in a 5 L Univessel Glass Biostat A bioreactors (Sartorius) with an initial medium volume of 3 litres. Culture growth dynamic was monitored by optical density measurements at 600 nm wavelength. The samples from bioreactors were taken at different process stages and analyzed. The bioreactors seeded with overnight inoculum (seeding volume calculated to achieve target seeding optical density of 0.15 OU). The initial culture parameters before target protein and simultaneous lysis induction were as follows: the dissolved oxygen (DO) control was maintained at 30% by automatic cascade: air flow (from 1.5 to 4.8 Lpm); the stirrer rate (from 420 to 800 rpm); pure oxygen flow (from 0 to 4.8 Lpm). Culture pH was controlled at 7.0±0.1 by automatic addition of NH₄OH (25%) or H₃PO₄ (42.5%). The growth temperature before MuLV-RT induction (MuLV-RT induction point: 7.0±0.5 OD) was maintained at 37° C. After induction with 0.1 mM IPTG the temperature in all processes was reduced from 37 to 28° C. over 40 minutes time span. After IPTG induction DO profile was started: 30% to 3% over 40 minutes. 6 h after IPTG induction lysozyme synthesis were induced by adding 0.5 g/L of arabinose. To perform final lysis, 1 h after lysozyme induction, osmotic shock was initiated by adding 1M sucrose, 0.5M Na₂SO₄ and 0.5% (w/v) brij-24 directly to the vessel. After 20 minutes pH and DO controllers were turned off, stirrer setpoint was set at 200 rpm. Mixing was performed overnight (14±2 hours). After osmotic shock phase harvesting/clarification steps were initiated.

### Clarification of the Fermentation Culture

To clarify the fermentation culture PEI (Lupasol, Polyethyleneimine, BASF) is added to the bioreactor to a final concentration of 0.25% (v/v) and mixing continued for 15-20 mins at 200-300 rpm. Material is transferred into separate tanks from bioreactor and mixed with water at a ratio 2:4-2:5 (v/v) at 200-300 rpm for 10-20 mins. Culture is left for sedimentation for 2-4 hours. After sedimentation the upper part of culture is clarified by filtering through the depth filtration capsules Supracap100 P050 (ID: NP5LP0501). After depth filtration, tangential flow filtration is performed using 0.3 m² TFF cassette (ID: OS030T12) with concentration factor 10×.

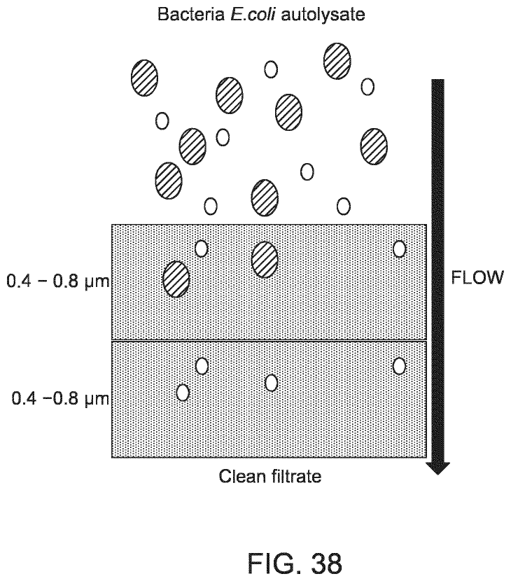

In more detail, the clarification step involved the following (FIG. 38):

Starting material: 250 L of cell lyzed cell culture, after 16-18 h of target protein expression in the stirred tank reactors. Final OD of the cells/cell debris ~15-30 OD. Analysis showed that induced cell lysis resulted in 75-80% cell lysis efficiency from total cell amount.

During all manipulations within stirred tack bioreactor (Hyperforma™ 300 L, Thermofisher Scientific) all automatic controls in fermentation vessel: Air/O2 flow, pH was in “off mode”. The stirring was maintained at 375 rpm and the temperature was set <12° C.

Enzyme stabilization and flocculation of nucleic acids. Dry materials were added directly in to the bioreactor vessel, one by one to achieve the final concentration of 1M Sucrose, 0.5M Na2SO4 and 0.5% Brij 35. High viscosity solution was mixed thoroughly at 375 rpm for 60 mins <12° C. After, the stirring was maintained at 100 rpm for 10-18h <12 C°. The flocculation was performed by addition of PEI (Lupasol, Polyethyleneimine, BASF) to the bioreactor vessel to achieve final concentration of 0.25% (v/v). Stirring was mixing continued for at least 15 mins at 375 rpm. The solution was transferred into separate tanks from bioreactor, diluted with water (molecular biology grade) by 2.5-fold, mixed at 200-300 rpm for 10-20 mins. After that mixing was stopped completely. Culture was left for sedimentation for ~3 hours.

Clarification: Filter preparation. Required amount of STAX filter capsules K050P (Pall, ID 7007786) were washed with 10 filter volumes of water. Without exceeding back pressure of 0.2 BAR of the pressure. After sedimentation the upper part of culture was clarified by filtering through the depth filtration capsulesK050P (Pall, ID 7007786) Retention 0.4-0.8 μM (about 0.05 m² of filter area is needed to filtrate 1 L of fermentation media) without exceeding back pressure of 1.2 BAR and subsequently was filtered through 0.22 μm filter capsule (Pall, ID NP6UEAVP1S). The secondary clarification was carried out using 0.22 μm filter capsule (Pall, ID NP6UEAVP1S). The same clarification process could be performed using filtration systems: 1-3 μM (P100) (Pall, ID 7007836).

### Results and Discussion

E. coli is not a perfect host especially for closed single use fermentation process because it normally does not secrete proteins into the extracellular medium. The cell disruption step becomes a limitation and is not cost effective at the pilot or industrial scale when the goal is the implementation of closed and/or continues process flow. The present invention is an approach for cell disruption that is a programmed cell lysis system based on the expression of a cloned T4 bacteriophage gene gpe to produce a lytic protein. Gene gpe encodes a lysozyme which degrades the peptidoglycan of E. coli cell wall.

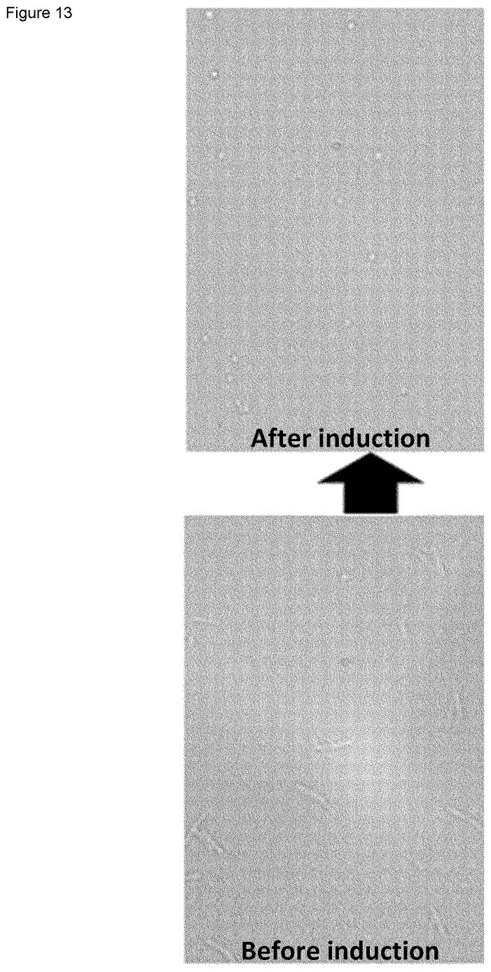

Self-disruptive Escherichia coli that produces foreign target protein was developed. E. coli JS007 was co-transformed with two vector plasmids a target gene expression vector pMuIV-RT-Km and a lysis gene expression vector pAra-pelB-gpe. The lytic protein was produced after the expression of the target gene, resulting in simplification of the cell disruption process. In this Example, the expression of cloned T4 phage gene gpe was used for the disruption of E. coli that produced MuLV-RT as a target protein. The expression of gene gpe did not lead to prompt cell disruption but weakened the cell wall. The translocation of lytic protein Gpe from the cytoplasm to the periplasm facilitated enzymatic degradation of murein layer which is responsible for maintaining cell strength. Microscopic observation revealed that cells producing Gpe in the periplasmic space after 1 h went through a morphological change, from rod-shaped to elliptical spheroplasts (FIG. 13).

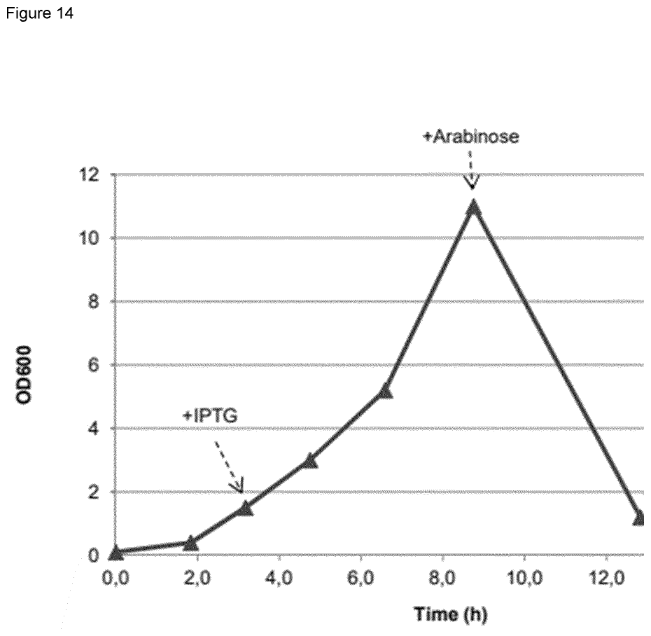

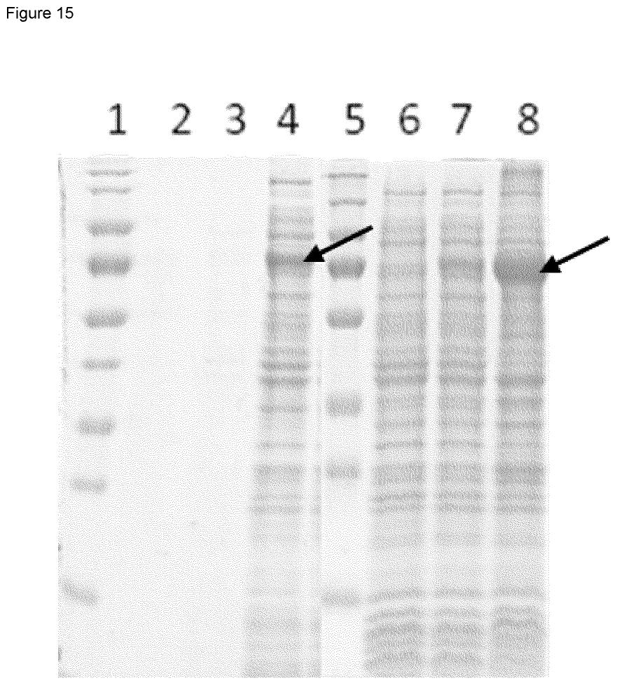

After 2 hour induction 0.5 g/l L-arabinose of E. coli JS007 pMuLV-RT-Km, pAra-pelB-gpe optical density decreased (FIG. 14) and debris of E. coli cells were observed in the culture medium, indicating that cell lysis had occurred. 12% SDS-PAGE showed that intracellular proteins were released to the culture medium and MuLV-RT protein was identified in biomass and culture medium samples (FIG. 15).

The batch MuLV-RT production processes in Biostat A stirred bioreactor were designed with the initial medium volume of 3 L. As shown in FIG. 16 the temperature of growth before MuLV-RT induction (MuLV-RT induction point: 7.0±0.5 OD) was maintained at 37° C. After target protein induction with 0.1 mM IPTG (MuLV-RT induction point: 7.0±0.5 OD) the temperature in all processes was reduced from 37 to 28° C. over 40 minutes time span and DO profile was started: 30% to 3% over 40 minutes. 6 h after IPTG induction lysozyme synthesis were induced by adding 0.5 g/L of arabinose. To perform final lysis, 1 h after lysozyme induction, osmotic shock was initiated by adding 1M sucrose, 0.5M Na₂SO₄ and 0.5% (w/v) brij-24 and left over night.

Depth filtration-based clarification of cell debris without nuclease treatment. Here, the best blend of high ionic strength creating salts, osmotic agent and detergent were used to stabilize enzyme, prevent precipitation/aggregation, prevent interaction with highly hydrophobic lysozyme and membrane proteins. Also flocculation of nucleic acid was used to create a filtration cake, comprised of small flocculent particles. It is demonstrated successfully using unusually pores of 0.4-08 um filters. Also it works with 1-3 um pore filters. E. coli cell size is: 1.0-2.0 micrometres long, with radius about 0.5 micrometres. Primary clarification approaches which are usually recommended for E. coli are tangential flow filtration clarification <0.2 micrometres pore size or centrifugation, which is the most common approach for clarification of cellular debris, large or small flocculants.

### Example 4—Expressing Recombinant Ribonuclease

### Engineering of Ribonuclease Inhibitor Gene.

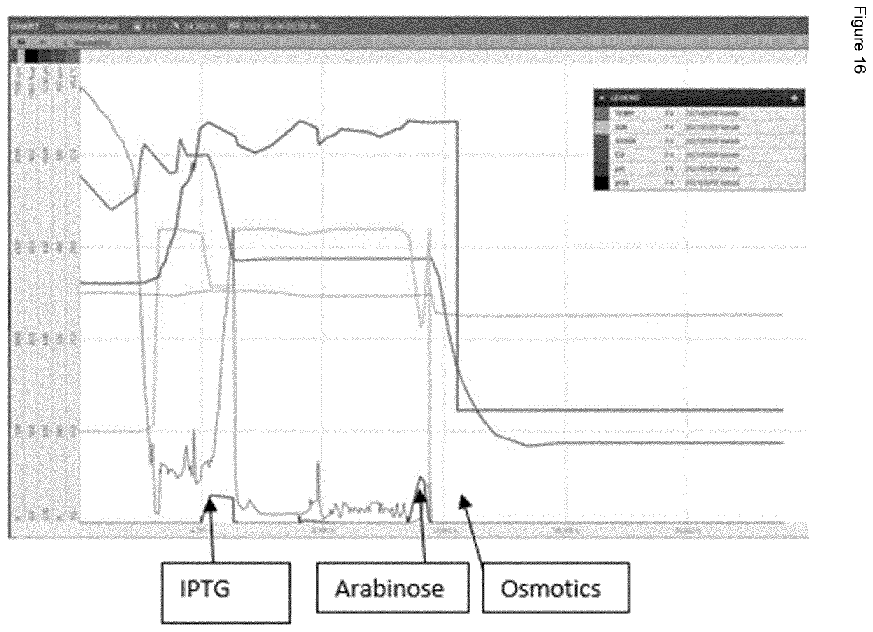

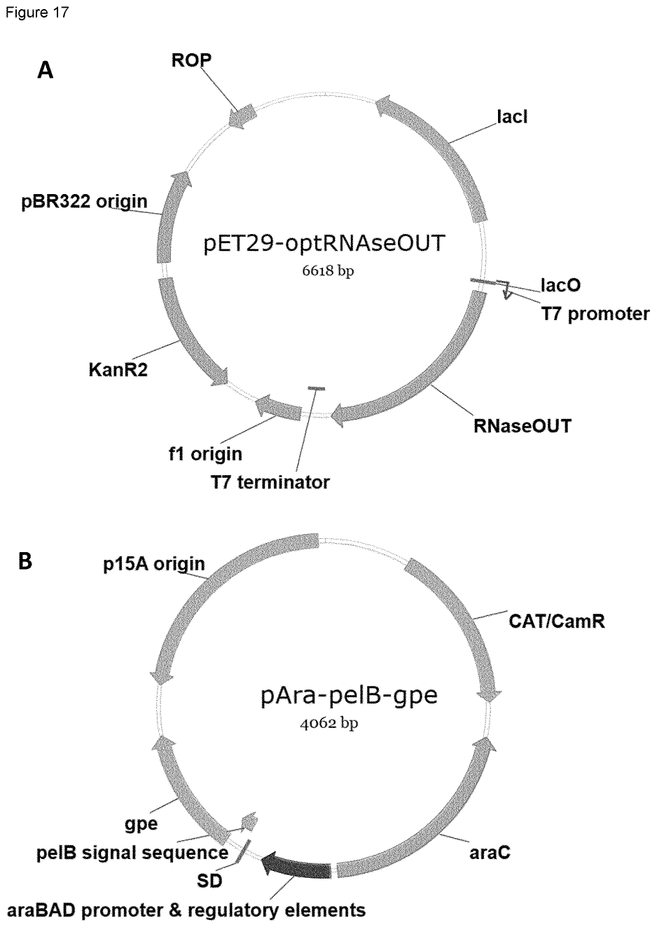

Optimized for expression in E. coli, ribonuclease inhibitor gene was cloned into pET29 vector under an inducible promoter PT7 (FIG. 17A). NCBI GeneID: 445517, protein sequence ID: XP_020938200.1, protein name: Ribonuclease Inhibitor. The ribonuclease inhibitor sequence is disclosed herein as SEQ ID NO:5.

### Engineering of T4 Bacteriophage Lysozyme Gene Gpe

T4 bacteriophage lysozyme gene gpe with signal peptide of pelB was cloned in to pAra vector under araB promoter (plasmid pAra-pelB-gpe). The plasmid pAra-pelB-gpe (FIG. 17B) carries an origin of replication derived from pACYC and a chloramphenicol-resistance gene gene which allows the use of E. coli expression systems containing ColE1-type plasmids.

In this example, an engineered polypeptide referred to as Gpe which sequence originated from lysozyme of Escherichia coli T4 bacteriophage was employed. NCBI Reference Sequence: NP_049736.1, protein name: glycoside hydrolase family protein [Escherichia phage T4]. PelB signal sequence: 1-22, T4 bacteriophage lysozyme 23-164. The protein sequence of gpe with pelb signal sequence is disclosed herein as SEQ ID NO:1.

### Expression Strain Preparation

The E. coli strain JS008 (Thermo Fisher Scientific) was transformed with the plasmids pET29-optRNAseOUT and pAra-pelB-gpe (carrying the gene for the T4 bacteriophage lysozyme, gpe) and then plated on LB (animal-origin free) AOF agar with kanamycin (50 g/l) and chloramphenicol (25 g/l). Transformants with both plasmids were plated on LB agar containing kanamycin (50 mg/l) and chloramphenicol (25 mg/l). The transformation was based on the calcium temperature shock method. A Research cell bank (RCB) was produced after 3 h of cultivation of the transformants in liquid TB4 AOF medium with the required antibiotics at 37° C. and 220 rpm. A 50% sterile glycerol solution was used to produce 25% glycerol RCB which were aliquoted in cryovials and stored at −70° C.

### Cultivation Media

Transformations and plasmid propagations were performed on solid and liquid LB AOF medium containing Vegetable peptone (10 g/L), Yeast extract (5 g/l), NaCl (10 g/l), and for solid medium 15 g/l microbiological agar, as well as the required antibiotics. Fed-batch and batch cultivation was performed in glycerol-based animal origin-free benzo medium (benzo w/o tryptone AOF and benzo AOF medium). The composition of benzo w/o tryptone AOF medium was as follows (per litre): yeast extract 5 g, NH₄Cl 1.5 g, (NH₄)₂SO₄ 2.7 g, KH₂PO₄ 6 g, K₂HPO₄ 4 g, glycerol 10 g. The composition of benzo AOF medium was as follows (per litre): vegetable peptone 24 g, yeast extract 24.5 g, NH₄Cl 1.5 g, (NH₄)₂SO₄ 2.7 g, KH₂PO₄ 6.1 g, K₂HPO₄ 4.08 g, glycerol 10 g. Additionally, before cultivation the benzo AOF medium was supplemented with the following sterile solutions: 3 mL of (1M) MgSO₄ and 3 mL of trace element solution with the following composition (per litre): CaCl₂×2H₂O 0.5 g, ZnSO₄×7H₂O 0.18 g, MnSO₄×H₂O 0.1 g, Na₂-EDTA 20.1 g, FeCl₃×6H₂O 16.7 g, CuSO₄×5H₂O 0.16 g, CoCl₂×6H₂O 0.18 g; as well as 100 μL L-1 of thiamine hydrochloride (1 M), 1 mL 5 of kanamycin (50 mg/ml) and 1 mL of chloramphenicol (25 mg/ml).

### Expression and Autolysis Conditions in Shake Flask

The inoculums for batch protein production in the shake flasks were prepared by overnight cultivation of the selected clone in 2000 mL shake flasks with 500 ml of Benzo w/o Tryptone medium containing 10 g/l of vegetable glycerol at 37° C. For protein production the corresponding inoculum culture (0.1 start OD600) was transferred to fresh Benzo AOF medium containing 10 g/l of vegetable glycerol to a final volume of 50 mL in 250 ml baffled Erlenmeyer shake flasks. Cultures were cultivated at 37° C. and 220 rpm until they reached a cell density of OD600 1.5-2.0. Induction was performed with 0.1 mM IPTG. The temperature was changed at the induction point to 25° C. and the culture was continued for 3 hours at 220 rpm. Afterwards, lysis was induced with a 125 μl of 20% solution of L-arabinose, the culture was incubated in the same conditions for 0.5 h more, followed by osmotic shock via the addition of 18.23 g sucrose, 3.79 g Na₂SO₄ and 0.27 g Brij™ 35. The flasks were then incubated overnight at 12° C., 100 rpm.

### Expression and Autolysis Conditions in Bioreactor

The inoculum for fed-batch protein production in the shake flask was prepared by overnight cultivation of the RCB in 2000 mL shake flaks with 500 ml of BenzoAOF medium containing 10 g/l of glycerol at 25° C. 180 rpm. Batch phase was performed in a 5 L Univessel Glass Biostat A bioreactors (Sartorius) with an Benzo AOF medium with 20 g/L glycerol volume of 3 litres. Culture growth dynamic was monitored by optical density measurements at 600 nm wavelength. The samples from bioreactors were taken at different process stages and analyzed. The bioreactors seeded with overnight inoculum (amount seeded is calculated by target of optical density of 0.15 ou). The initial culture parameters before target protein and simultaneous lysis induction were as follows: the dissolved oxygen (DO) control was maintained at 30% by automatic cascade: air flow (from 1 to 4.8 Lpm); the stirrer rate (from 420 to 800 rpm); pure oxygen flow (from 0 to 4.8 Lpm). Culture pH was controlled at 7.0±0.1 by automatic addition of NH₄OH (25%) or H₃PO₄ (42.5%). The growth temperature before ribonuclease inhibitor induction (induction point: 5.0-6.6 OD) was maintained at 37° C. After induction with 0.2 mM IPTG the temperature in all processes was reduced from 37 to 25° C. 1 h after IPTG induction DO profile was started: 30% to 3% over 20 minutes. 3 h after IPTG induction lysozyme synthesis were induced by adding 0.5 g/L of arabinose. To perform final lysis, 3 h after lysozyme induction, osmotic shock was initiated by adding sucrose to a final concentration of 1M, sodium sulfate to a final concentration of 0.5M and Brij-35 to a final concentration of 0.5% (w/v), DTT is added to 1 g/L of culture medium and everything is mixed at 375 rpm for 20-30 mins and then 10-15 h at 100 rpm at 12° C.

### Analytical Tools

1 ml culture samples were harvested from flasks, 1 Pierce™ Universal Nuclease for Cell Lysis is added to 1 ml cell culture and centrifuged for 1 min at 13000 rpm. The media fraction was then used for further analysis—40 μl of media fraction had 50 μl of 2× Tris-Glycine SDS sample buffer added, followed by 10 μl of 10× Sample Reducing Agent to obtain a final sample volume of 100 μl. Samples were heated for 10 min at 95° C. 5 μL of sample was applied to each lane of a 12% SDS-PAGE gel.

### Clarification of the Fermentation Culture

PEI (Lupasol, Polyethyleneimine, BASF) is added to the bioreactor to a final concentration of 0.15% (v/v) and mixing continued for at least 15 mins at 375 rpm. Material is transferred into separate tanks from bioreactor and mixed with water at a ratio 1:1 (v/v) at 200-300 rpm for 10-20 mins. Culture is left for sedimentation for 180-240 mins. After sedimentation the upper part of culture is clarified by filtering through the depth filtration capsules K050P (Pall, ID 7007786) and subsequently is filtered through 0.22 μm filter capsule (Pall, ID NP6UEAVP1S).

For downstream applications buffer solution in clarified media was exchanged by ultrafiltration. Ultrafiltration was performed by tangential flow filtration system (TFF) using TFF filter with pore size of 30 kDa filter. Filtration area was 0.01 m² for 1 L media solution.

In more detail, the clarification process involved the following (FIG. 38):

Starting material: 250 L of cell lysed cell culture, after 16-18 h of target protein expression in the stirred tank reactors. Final OD of the cells/cell debris ~15-20 OD. Analysis showed that induced cell lysis resulted in 75-80% cell lysis efficiency from total cell amount.

During all manipulations within stirred tack bioreactor (Hyperforma™ 300 L, Thermofisher Scientific) all automatic controls in fermentation vessel: Air/O2 flow, pH was in “off mode”. The stirring was maintained at 375 rpm and the temperature was set <12° C.

Enzyme stabilization and flocculation of nucleic acids. Dry materials were added directly in to the bioreactor vessel, one by one to achieve the final concentration of 1M Sucrose, 0.5M Na2SO4 and 0.5% Brij 35. High viscosity solution was mixed thoroughly at 375 rpm for 60 mins <12° C. After, the stirring was maintained at 100 rpm for 10-18h <12 C°. The flocculation was performed by addition of PEI (Lupasol, Polyethyleneimine, BASF) to the bioreactor vessel to achieve final concentration of 0.25% (v/v). Stirring was mixing continued for at least 15 mins at 375 rpm. The solution was transferred into separate tanks from bioreactor, diluted with water (molecular biology grade) by 2.5-fold, mixed at 200-300 rpm for 10-20 mins. After that mixing is stopped completely. Culture is left for sedimentation for ~3 hours.

Clarification: Filter preparation. Required amount of STAX filter capsules K050P (Pall, ID 7007786) were washed with 10 filter volumes of water. Without exceeding back pressure of 0.2 BAR of the pressure. After sedimentation the upper part of culture was clarified by filtering through the depth filtration capsulesK050P (Pall, ID 7007786) Retention 0.4-0.8 μM (about 0.05 m² of filter area is needed to filtrate 1 L of fermentation media) without exceeding back pressure of 1.2 BAR and subsequently was filtered through 0.22 μm filter capsule (Pall, ID NP6UEAVP1S). The secondary clarification was carried out using 0.22 μm filter capsule (Pall, ID NP6UEAVP1S). The same clarification process could be performed using filtration systems: 1-3 μM (P100) (Pall, ID 7007836).

### Results and Discussion

E. coli is not a perfect host especially for closed single use fermentation process because it normally does not secrete proteins into the extracellular medium. The cell disruption step becomes a limitation and is not cost effective at the pilot or industrial scale when the goal is the implementation of closed and/or continues process flow. The present approach for cell disruption is the programmed cell lysis system based on the expression of a cloned T4 bacteriophage gene gpe to produce a lytic protein. Gene gpe encodes a lysozyme which degrades the peptidoglycan of E. coli cell wall.

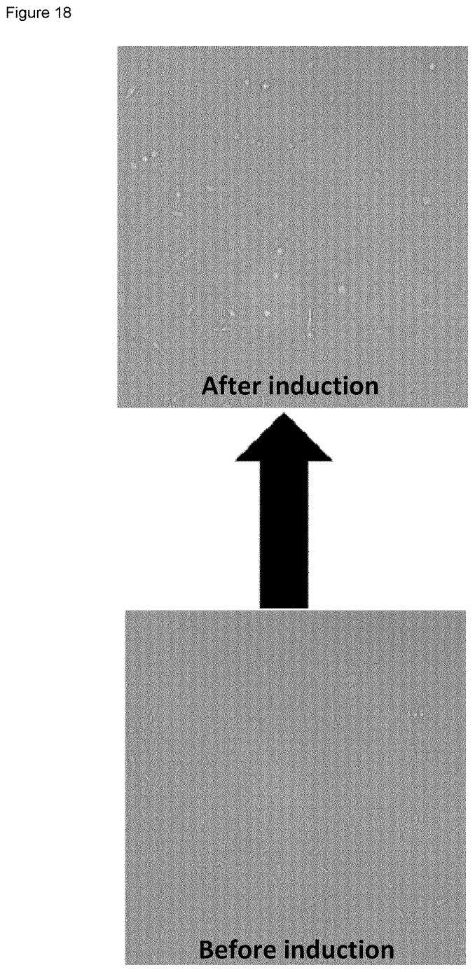

Self-disruptive Escherichia coli that produces Ribonuclease Inhibitor was developed. E. coli JS008 was co-transformed with two vector plasmids a target gene expression vector pET29-optRNAseOUT and a lysis gene expression vector pAra-pelB-gpe. The lytic protein was induced after 3 hours of the expression induction of the target gene, by adding arabinose to final concentration 0.005% resulting in simplification of the cell disruption process. In this example, the expression of cloned T4 phage gene gpe was used for the disruption of E. coli that produced Ribonuclease Inhibitor as a target protein. The expression of gene gpe did not lead to prompt cell disruption but weakened the cell wall. The translocation of lytic protein Gpe from the cytoplasm to the periplasm facilitated enzymatic degradation of murein layer which is responsible for maintaining cell strength. Additionally, osmotic shock was performed after 0.5 hour after lysis protein induction in this example in order to facilitate releasing of goal protein into growing media. After 20 hours of induction of E. coli JS008 pET29-optRNAseOUT pAra-pelB-gpe Ribonuclease Inhibitor was released to the culture medium. Microscopic observation revealed that cells producing Gpe in the periplasmic space went through a morphological change, from rod-shaped to elliptical spheroplasts (FIG. 18).

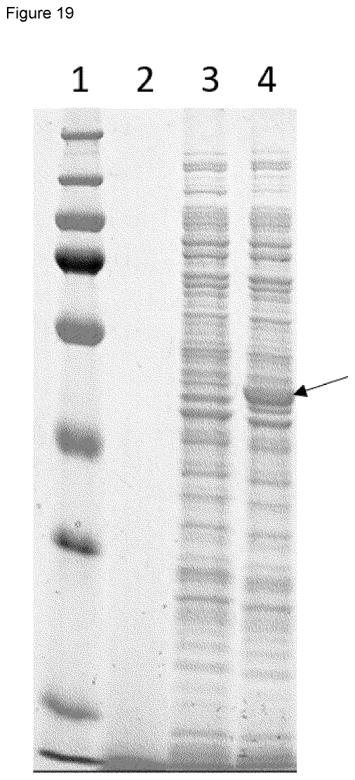

Debris of E. coli cells were observed in the culture medium, indicating that cell lysis had occurred. Maximum Ribonuclease Inhibitor production in shake flask was obtained after overnight expression of E. coli JS008 pET29-optRNAseOUT, pAra-pelB-gpe at 12° C. when lysozyme induction with arabinose was performed 3 hours after target gene induction. T7 polymerase protein expression was identified by the SDS PAGE (FIG. 19). E. coli JS008 pET29-optRNAseOUT, pAra-pelB-gpe culture media sample after Ribonuclease Inhibitor induction were taken for gel electrophoresis analysis. Culture media sample without induction (3) as well media without bacteria (2) were taken as negative controls. The protein band in sample (4) between 35 kDa and 55 kDa ladder bands clearly indicate presence of Ribonuclease Inhibitor protein (~49 kDa) in growing media (indicated by the black arrow).

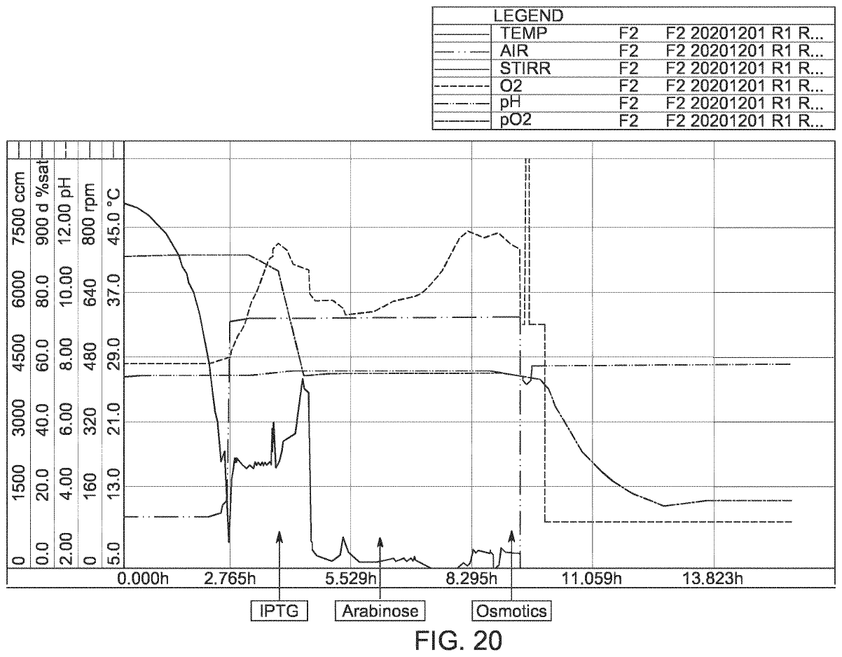

The batch RI production processes in Biostat A stirred bioreactor were designed with the initial medium volume of 3 L. As shown in FIG. 20 the temperature of growth before RI induction (induction point: 5.0-6.6 OD) was maintained at 37° C. After induction with 0.2 mM IPTG the temperature in all processes was reduced from 37 to 25° C. over 40 minutes time span. 1 h after IPTG induction DO profile was started: 30% to 3% over 20 minutes. 3 h after IPTG induction lysozyme synthesis were induced by adding 0.5 g/L of arabinose. To perform final lysis, 3 h after lysozyme induction, osmotic shock was initiated.

To compare repeatability and examine growth rate fermentation processes from RCB and fresh transformation were performed side by side in Biostat A bioreactors. Growth rate was determined by periodical optical density measurements which are shown in FIG. 21. Results revealed that both transformant and RCB have similarities in culture growth rate.

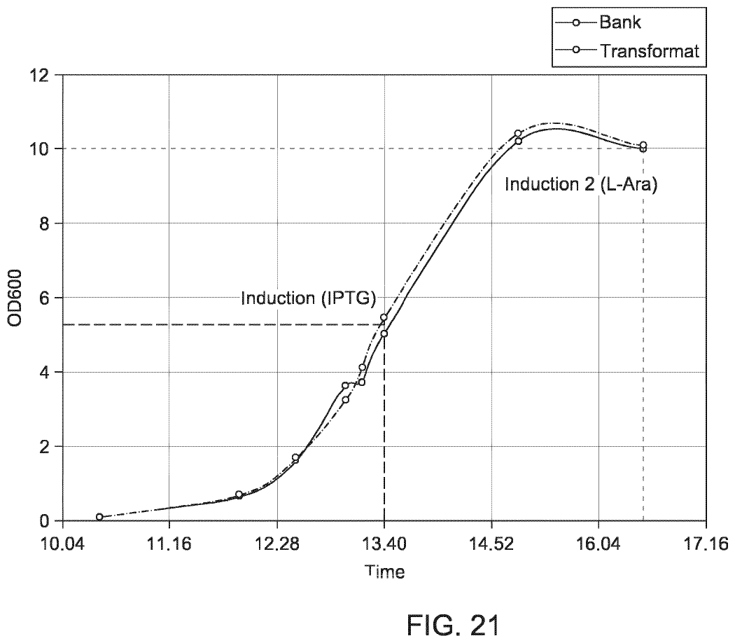

Biosynthesis parameters that were used in development were downscaled from 300 L stainless steel fermenter capabilities down to 5 L Univessel Glass Biostat A bioreactors in order to ensure that optimized process would be transferrable to manufacturing facilities. Optimized process parameters were evaluated based on target protein yield. The success of autolysis was further analyzed by running samples on agarose gel as shown in FIG. 22.

Depth filtration-based clarification of cell debris without nuclease treatment. Here, the best blend of high ionic strength creating salts, osmotic agent and detergent were used to stabilize enzyme, prevent precipitation/aggregation, prevent interaction with highly hydrophobic lysozyme and membrane proteins. Also flocculation of nucleic acid was used to create a filtration cake, comprised of small flocculent particles. It is demonstrated successfully using unusually pores of 0.4-08 um filters. Also it works with 1-3 um pore filters. E. coli cell size is: 1.0-2.0 micrometres long, with radius about 0.5 micrometres. Primary clarification approaches which are usually recommended for E. coli are tangential flow filtration clarification <0.2 micrometres pore size or centrifugation, which is the most common approach for clarification of cellular debris, large or small flocculants.

### Example 5—Expressing Recombinant Vaccinia Virus 2-O-Methyltransferase (OMT)

### Engineering of Vaccinia Virus 2-O-Methyltransferase Gene Vp39

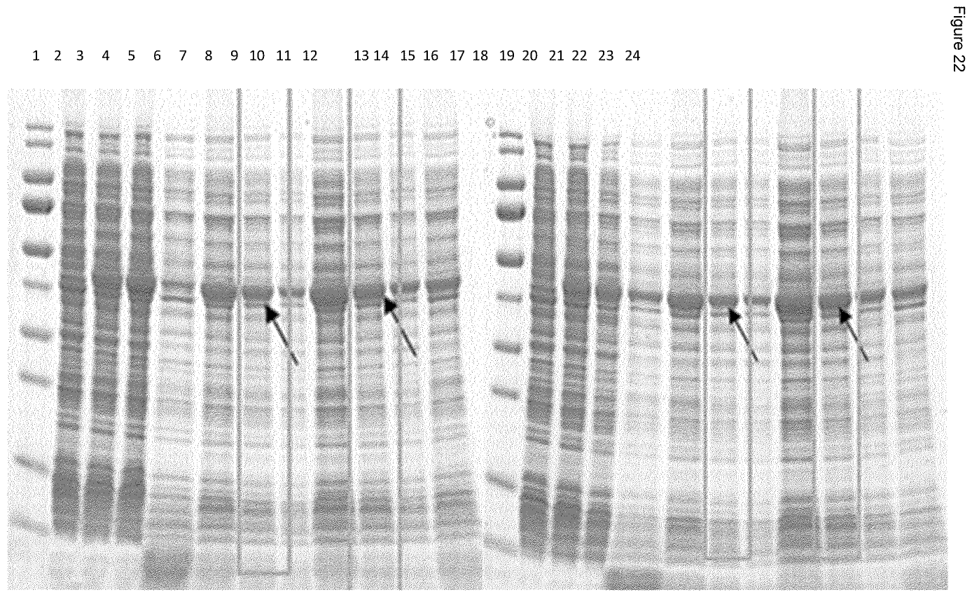

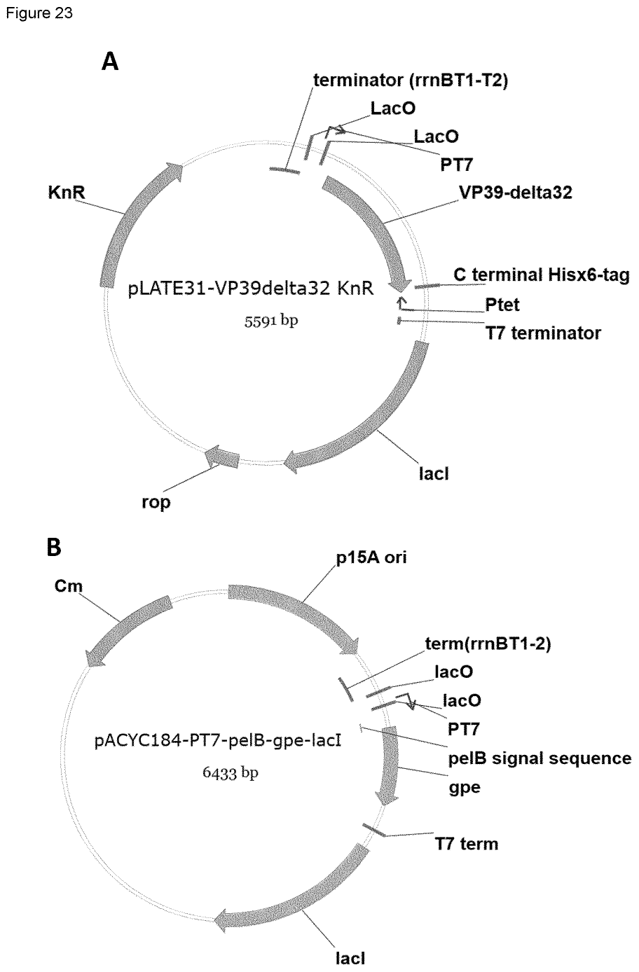

Optimized for expression in E. coli vp39 gene was cloned in to pLATE31 vector (Thermo Fisher) (FIG. 23A). NCBI Sequence ID: AGJ91263.1, protein name: multifunctional Poly-A polymerase-small subunit VP39 [Vaccinia virus]. The 2-O-methyltransferase amino acid sequence used in this example is disclosed herein as SEQ ID NO:6.

### Engineering of T4 Bacteriophage Lysozyme Gene Gpe

T4 bacteriophage lysozyme gene gpe was cloned in to tight controlled pLATE11 vector (Cat. K1241, Thermo Fisher) to reduce the expression of lysis genes in uninduced cells. In additional, the signal peptide of pelB to translocate the produced protein to the periplasmic space was fused at the N-terminus of Gpe. The fragment containing the transcription terminator rrnBT1-T2 prevented basal gene expression from vector derived promoter-like elements, the T7 RNA polymerase promoter, which drives transcription of the cloned gpe gene, and two flanking lac operator sequences ensure tight control of gene expression, the Ptet promoter reduces basal expression from the T7 promoter, the T7 terminator which terminates transcription from the T7 promoter and the lac repressor which ensures tight control of basal expression from the T7 promoter was subcloned into the pACYC184 vector that have a p15A origin of replication and is compatible with plasmids harboring ColE1 on (plasmid pACYC184-PT7-pelB-gpe-lacl) (FIG. 23B).

For these experiments, engineered polypeptide referred to as Gpe which sequence originated from lysozyme of Escherichia coli T4 bacteriophage was used. NCBI Reference Sequence: NP_049736.1, protein name: glycoside hydrolase family protein [Escherichia phage T4]. PelB signal sequence: 1-22, T4 bacteriophage lysozyme 23-164. The protein sequence of Gpe with PelB signal sequence is disclosed herein as SEQ ID NO:1.

### Expression Strain Preparation

The E. coli strain JS007 (Thermo Fisher Scientific) was transformed with the plasmid pLATE31-VP39delta32-KnR and was plated on LB (animal-origin free) AOF agar with kanamycin (50 g/l). The expression strain E. coli JS007 pLATE31-VP39delta32-KnR was co-transformed separately with the lysis plasmid pACYC-PT7-pelB-gpe-lacl, carrying the gene for the T4 bacteriophage lysozyme gene gpe. Transformants with both plasmids were plated on LB agar containing kanamycin (50 mg/l) and chloramphenicol (25 mg/l). Both transformations were based on the calcium temperature shock method. Research cell bank (RCB) was produced after 3 h of cultivation of the transformants in liquid TB4 AOF medium with the required antibiotics at 37° C. and 220 rpm. A 50% sterile glycerol solution was used to produce 25% glycerol RCBs which were aliquoted in cryovials and stored at −70° C.

### Cultivation Media

Transformations and plasmid propagations were performed on solid and liquid LB AOF medium containing peptone (10 g/L), Yeast extract (5 g/l), NaCl (10 g/l), and for solid medium 15 g/l microbiological agar, as well as the required antibiotics. Fed-batch and batch cultivations were performed in glycerol-based animal origin free (AOF) terrific broth (TB4 AOF and TB10 AOF) with the following composition (per liter): peptone 12 g, Yeast extract 24 g, (NH₄)₂SO₄ 2.68 g, KH₂PO₄ 2.3 g, K₂HPO₄12.5 g and glycerol 4 to 10 g. Additionally, before cultivation the TB10 medium was supplemented with the following sterile solutions: 3 mL of (1M) MgSO₄ and 2 mL of trace element solution with the following composition (per liter): CaCl₂×2H₂O 0.5 g, ZnSO₄×7H₂O 0.18 g, MnSO₄×H₂O 0.1 g, Na₂-EDTA 20.1 g, FeCl₃×6H₂O 16.7 g, CuSO₄×5H₂O 0.16 g, CoCl₂×6H₂O 0.18 g; as well as 1 mL 5 of kanamycin (50 mg mL/I) and 1 mL of chloramphenicol (30 mg/l).

### Expression and Autolysis Conditions in Shake Flask

The inoculum for batch protein production in the shake flasks were prepared by overnight cultivation of the selected clone in 250 mL shake flask with 50 ml of TB4 AOF medium containing 4 g/l of vegetable glycerol at 37° C. For protein production the corresponding inoculum culture was transferred to fresh TB10 AOF medium containing 10 g/l of glycerol to a final volume of 50 mL in 250 ml baffled Erlenmeyer shake flasks. Culture was cultivated at 37° C. and 220 rpm until they reached a cell density of OD600 1.5-2.0. Induction was performed with 0.1 mM IPTG. The temperature was changed at the induction point to 32° C. and the culture was continued for 17 hours at 220 rpm.

### Expression and Autolysis Conditions in Bioreactor

Cell bank revival (pre-inoculum preparation) was performed by shake flask cultivation process in 250 ml type baffled Erlenmeyer flasks with 50 ml volume of TB4 AOF medium containing 4 g/L glycerol, 3 ml/L 1M MgSO₄, 2 ml/L Mkt-MSM, 50 mg/L kanamycin and 25 mg/L chloramphenicol at 37° C., 230 rpm for 4-6 h. Later inoculum seed volume calculated by formula 0.5/pre-inoculum OD was transferred to fresh 2000 ml type baffled Erlenmeyer flasks with 500 ml volume of TB4 AOF medium containing 4 g/L glycerol, 3 ml/L 1M MgSO₄, 2 ml/L Mkt-MSM, 50 mg/L kanamycin and 25 mg/L chloramphenicol for inoculum cultivation at 25° C., 260 rpm for 16-18 h.

Fed-batch fermentation was performed in 5 L Biostat A bioreactors with TB10 AOF medium containing 10 g/L glycerol, 3 ml/L 1M MgSO₄, 2 ml/L Mkt-MSM, 3 ml/L 10 mg/ml Thiamine-HCl, 50 mg/L kanamycin, 25 mg/L chloramphenicol and 0.2 ml/L Pluronic with an initial cultivation volume of 3 L. Actual inoculum seed volume required for bioreactors seeding was calculated by formula: (fermentation volume [ml]×inoculum target OD (0.1))/measured inoculum OD. The initial culture parameters as follows: temperature was maintained at 37° C., the pO2—at 30% by adapting the stirrer rate and automatic regulation of the air flow (from 500 to 1600 ccm), pH was controlled at 7.0±0.1 by addition of NH₄OH (25%) or H₃PO₄ (42.5%). Fed-batch phase was maintained with fed solution containing 60 g/L vegetable peptone solution, 60 g/L yeast extract, 10.57 g/L (NH₄)₂SO₄, 2 ml/L Mkt-MSM, 80 ml/L 1M MgSO₄ 400 g/L glycerol, 50 mg/L kanamycin and 25 mg/L chloramphenicol. Feed profile was set to maintain 20 ml/h/L medium feeding speed for total of 8 h with 13.5% pump speed. Protein expression and cell lysis induction was performed by adding IPTG solution (final conc. 0.1 mM) to the bioreactor after which followed temperature downregulation from 37 to 25° C. within period of 30 min and pO2 reduction from 30% to 3% over 30 min time span. Duration of protein expression and cell lysis induction was 18-22 h and through all the process optical density of the medium was measured at 600 nm (OD600) to estimate the cell concentration.

For better cell disruption osmotic shock was carried out by adding 71.01 g/L 0.5M Na₂SO₄ and 5 g/L 0.5% Brij-35. After osmotics addition pO2, pH, temperature and air flow automatic support were turned off and only 700 rpm mixing rate was kept. Later all bioreactors' suspensions were harvested and transferred to other groups for initiation of clarification steps.

### Assay of Protein Separation Using SDS-PAGE Analytical Technique

Cells and media fractions were separated by centrifugation for 30 min, 14000 rpm, 4° C. Media fractions were stored at 4° C. Cell samples harvested from flask cultivations were resuspended in lysis buffer at the following ratio: 1 g of biomass were resuspended in 10 mL of lysis buffer (50 mM Tris-HCl, 50 mM NaCl, 0.1 mM EDTA). The biomass was sonicated for 60 sec at 4° C. Soluble and insoluble protein fractions were separated by centrifugation for 20 min, 14000 rpm, 4° C. The total protein fraction represents cellular debris suspension (crude extract). Cell samples for SDS-PAGE separation were prepared as follows: 20 μL of crude extract sample, 25 μL of 4×SDS-PAGE loading buffer, 5 μL of 2M DTT and 50 μL of deionized water to obtain a final sample volume of 100 μL. Media samples for SDS-PAGE separation were prepared as follows: 70 μL of culture media, 25 μL of 4×SDS-PAGE loading buffer, 5 μL of 2M to obtain a final sample volume of 100 μL. Samples were heated for 10 min at 95° C. 10 μL of sample was applied to each lane of a 10% SDS-PAGE gels.

### Clarification of the Fermentation Culture

To clarify the fermentation culture, sodium sulfate was added to the bioreactor to a final concentration of 0.5M and Brij-35 to a final concentration of 0.5% (w/v) and all mixed at 250-350 rpm for 30-60 mins 32° C. Afterwards PEI (Lupasol, Polyethyleneimine, BASF) was added to the bioreactor to a final concentration of 0.25% (v/v) and mixing continued for at least 10 mins at 250-350 rpm. Material was transferred into separate tanks from the bioreactor and mixed with water at a ratio 2:5 (v/v) at 50-100 rpm for 10-20 mins. The culture was left for sedimentation for 3 hours to overnight. After sedimentation the upper part of the culture was clarified by filtering through the depth filtration capsules K050P (Pall, ID 7007786) and subsequently filtered through 0.22 μm filter capsule (Pall, ID NP6UEAVP1S).

In more detail the clarification process involved the following (FIG. 38):

E. coli autolyzate clarification process: starting material: 250 L of cell lysed cell culture, after 16-18 h of target protein expression in the stirred tank reactors. Final OD of the cells/cell debris ~15-30 OD. Analysis showed that induced cell lysis resulted in 75-80% cell lysis efficiency from total cell amount.

1.0 E. coli lysate preparation for filtration: during all manipulations within stirred tack bioreactor (Hyperforma™ 300 L, Thermofisher Scientific) all automatic controls in fermentation vessel: Air/O2 flow, pH. The stirring was maintained at 375 rpm and the temperature was set to <12° C.

Cell vessel disruption, enzyme stabilization and flocculation of nucleic acids. Dry materials were added directly in to the bioreactor vessel, one by one to achieve the final concentration of 1M Sucrose, 0.5M Na₂SO₄ and 0.5% Brij 35. High viscosity solution was mixed thoroughly at 375 rpm for 20-40 mins at 12° C. After, the stirring was maintained at 100 rpm for 10-18h <12 C°. The flocculation was performed by addition of PEI (Lupasol, Polyethyleneimine, BASF) to the bioreactor vessel to achieve final concentration of 0.25% (v/v). Stirring was mixing continued for at least 15 mins at 350 rpm. The solution was transferred into separate tanks from bioreactor, diluted with water (molecular biology grade) by 2.5-fold, mixed at 200-300 rpm for 10-20 mins. After that mixing was stopped completely. Culture is left for sedimentation for ~3 hours.

Clarification: Filter preparation. STAX filter capsules K050P (Pall, ID 7007786) was washed with 10 filter volumes of water. Without exceeding back pressure of 0.2 BAR of the pressure. After sedimentation the upper part of culture was clarified by filtering through the depth filtration capsules K050P (Pall, ID 7007786) Retention 0.4-0.8 μM (about 0.05 m² of filter area is needed to filtrate 1 L of fermentation media), without exceeding back pressure of 1.2 BAR. The secondary clarification was carried out using 0.22 μm filter capsule (Pall, ID NP6UEAVP1S). The same clarification process could be performed using filtration systems: 1-3 μM (P100) (Pall, ID 7007836).

### Results and Discussion

E. coli is not a perfect host especially for closed single use fermentation process because it normally does not secrete proteins into the extracellular medium. The cell disruption step becomes a limitation and is not cost effective at the pilot or industrial scale when the goal is the implementation of closed and/or continues process flow. The present approach for cell disruption is the programmed cell lysis system based on the expression of a cloned T4 bacteriophage gene gpe to produce a lytic protein. Gene gpe encodes a lysozyme which degrades the peptidoglycan of E. coli cell wall.

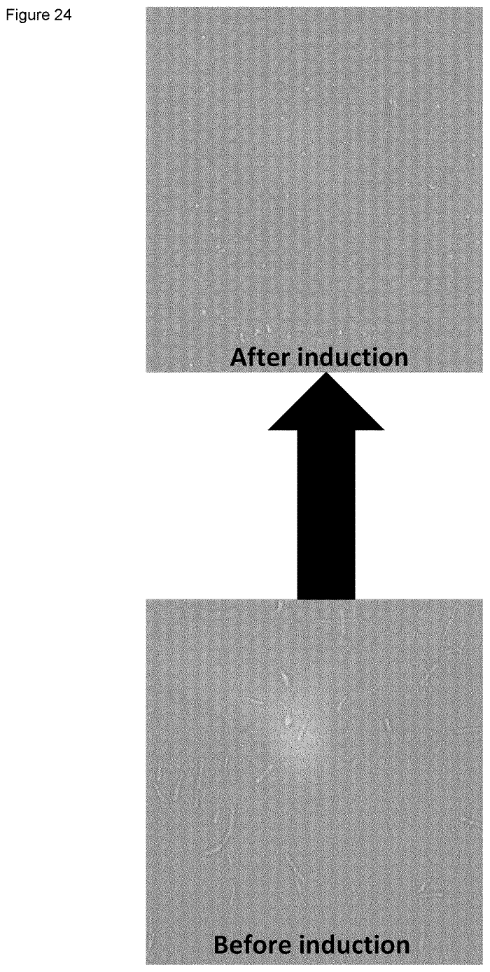

Self-disruptive Escherichia coli that produces foreign target protein was developed. E. coli JS007 was co-transformed with two vector plasmids a target gene expression vector pLATE31-VP39delta32-KnR and a lysis gene expression vector pACYC184-PT7-pelB-gpe-lacl. The lytic protein was produced after the expression of the target gene, resulting in simplification of the cell disruption process. In this example, the expression of cloned T4 phage gene gpe was used for the disruption of E. coli that produced OMT as a target protein. The expression of gene gpe did not lead to prompt cell disruption but weakened the cell wall. The translocation of lytic protein Gpe from the cytoplasm to the periplasm facilitated enzymatic degradation of murein layer which is responsible for maintaining cell strength. Microscopic observation revealed that cells producing Gpe in the periplasmic space after 5 h went through a morphological change, from rod-shaped to elliptical spheroplasts (FIG. 24).

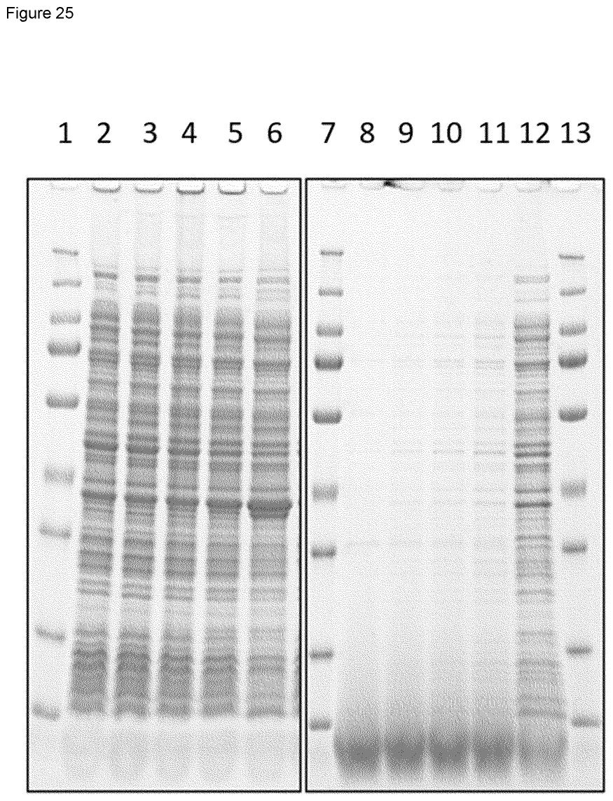

Induction of 2-O-methyltrasferase in JS007/pLATE31-VP39delta32-KnR/pACYC-PT7-pelB-gpe-lacl culture was performed with 0.1 mM IPTG. The induction of lysozyme gene was performed simultaneously. The culture were grown overnight at 32° C. Samples from inducted culture and media were taken 1, 2, 3, 5 and 17 hours after protein synthesis induction. Debris of E. coli cells were observed in the culture medium, indicating that cell lysis had occurred. Maximum 2-O-methyltrasferase yield in media was obtained after 17 hours expression/lysis of E. coli JS007/pLATE31-VP39delta32-KnR/pACYC-PT7-pelB-gpe-lacl strain. 2-O-methyltrasferase amount was identified by the SDS PAGE (FIG. 25).

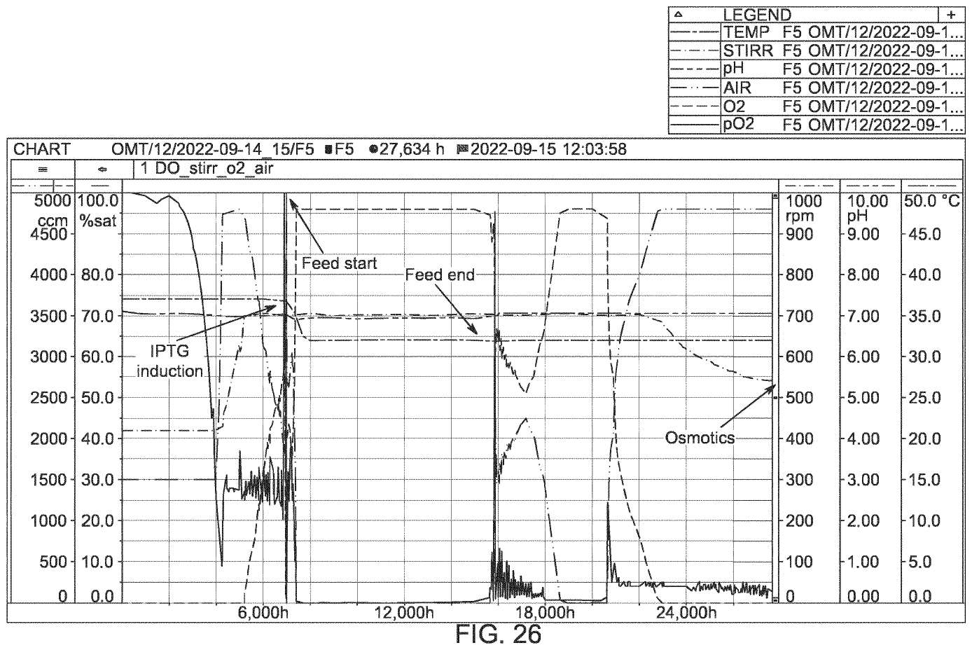

Fed-batch OMT production was performed in 5 L Biostat A bioreactors with the initial cultivation volume of 3 L. As shown in FIG. 26 protein expression and cell lysis induction was performed by adding IPTG solution (final conc. 0.1 mM) to the bioreactor at induction point target OD600 of 14-17.5. The fed-batch phase was started after DO spike which usually occurred 10-25 min after protein expression and lysis induction. Fed-Batch process was controlled automatically by process profile which maintained 20 ml/h/1 L medium feeding speed for total of 8 h with 13.5% pump speed. After 15 min of induction with IPTG the temperature was downregulated from 37 to 25° C. within period of 30 min while pO2 was reduced from 30% to 3% 1 h after induction with IPTG within period of 30 min. After protein expression and cell lysis induction phase (18-22 h) gas supply, pH and temperature control were turned off and only 700 rpm mixing rate was kept for osmotic shock fulfilment. After 30 min of osmotic shock all bioreactors' suspensions were harvested and clarification steps were initiated.

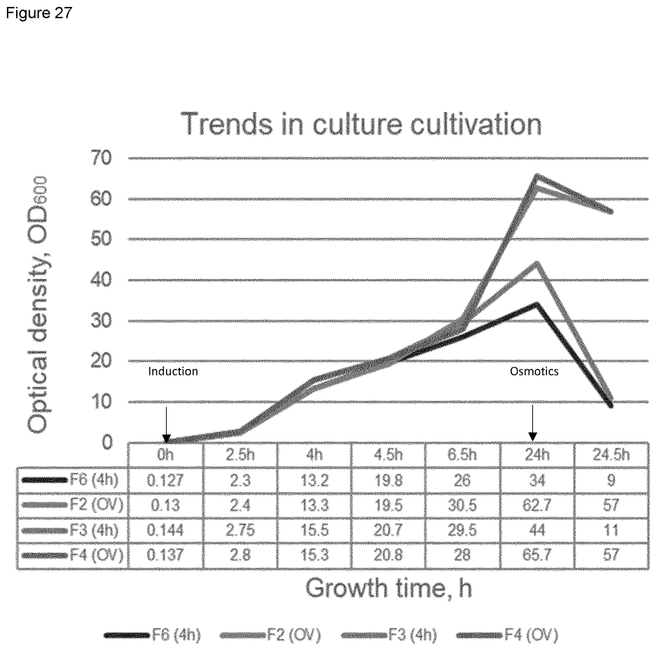

To compare repeatability and examine growth rate fermentation processes were performed side by side with Biostat A bioreactors. Growth rate was determined by periodical optical density measurements which are shown in FIG. 27. Results reveal that both processes have similarities in culture growth rate, but cultures from F2 and F4 with overnight feeding exceled higher optical densities at the end of process, though cultures from F6 and F3 with 4 h of feeding—higher activity with values of 43.7 and 55.0 U/pl.

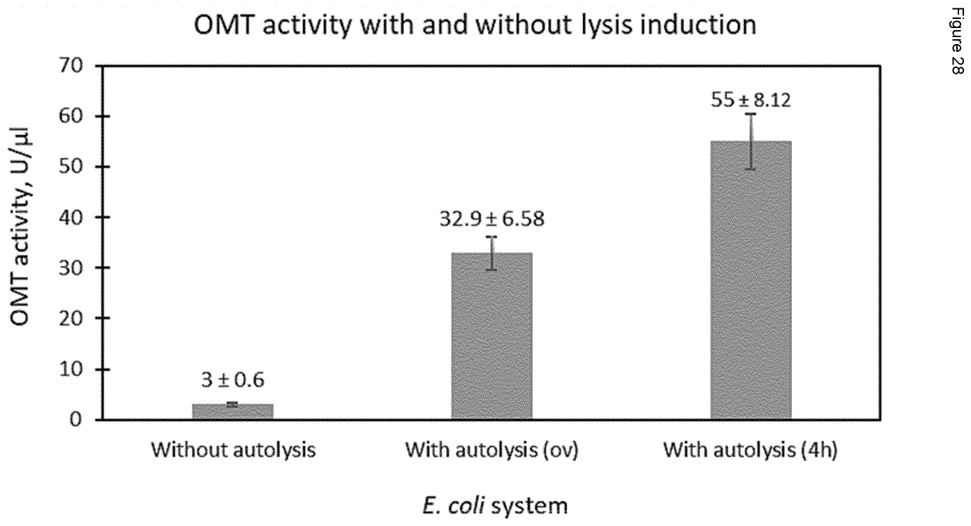

In order to examine how autolysis system and feeding time determines protein activity tendencies similar fermentation processes were also initiated. It was found that when using autolysis and fed-batch systems OMT yield increased 18 times as shown in FIG. 28, but extended feeding time slightly reduced it.

Biosynthesis parameters that were used in development were successfully upscaled from 5 L Univessel Glass Biostat A bioreactors to 300 L stainless steel fermenter capabilities to ensure that optimized process would be transferrable to manufacturing facilities. Optimized process parameters were evaluated based on target protein yield. By comparing old manufacturing technology results with new approach which features E. coli system with autolysis, it was found that with autolysis system OMT yield increased around 70 times—from 857 mg to 60000 mg (amount from 300 L bioreactor) after clarification and purification processes.

Depth filtration-based clarification of cell debris without nuclease treatment. Here, the best blend of high ionic strength creating salts, osmotic agent and detergent were used to stabilize enzyme, prevent precipitation/aggregation, prevent interaction with highly hydrophobic lysozyme and membrane proteins. Also flocculation of nucleic acid was used to create a filtration cake, comprised of small flocculent particles. It was demonstrated successfully using unusually pores of 0.4-08 um filters. Also it worked with 1-3 um pore filters. E. coli cell size is: 1.0-2.0 micrometres long, with radius about 0.5 micrometres. Primary clarification approaches which are usually recommended for E. coli are tangential flow filtration clarification <0.2 micrometres pore size or centrifugation, which is the most common approach for clarification of cellular debris, large or small flocculants.

### Example 6—Expressing Recombinant Cas9

### Engineering of Cas9 Nuclease Gene

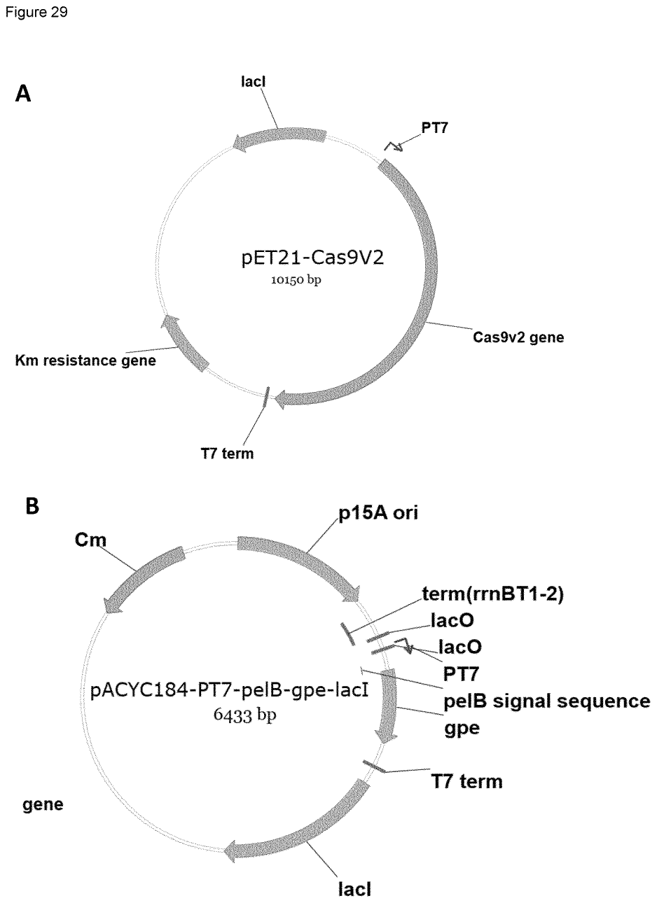

Protein name: Cas9 V2. origin of target gene: Streptococcus pyogenes. The cas9 amino acid sequence was cloned as shown in FIG. 29A.

### Engineering of T4 Bacteriophage Lysozyme Gene Gpe

T4 bacteriophage lysozyme gene gpe was cloned into tight controlled pLATE11 vector (Cat. K1241, Thermo Fisher) to reduce the expression of lysis genes in uninduced cells. In additional, the signal peptide of pelB to translocate the produced protein to the periplasmic space was fused at the N-terminus of Gpe. The fragment containing the transcription terminator rrnBT1-T2 prevented basal gene expression from vector derived promoter-like elements, the T7 RNA polymerase promoter, which drives transcription of the cloned gpe gene, and two flanking lac operator sequences ensure tight control of gene expression, the Ptet promoter reduces basal expression from the T7 promoter, the T7 terminator which terminates transcription from the T7 promoter and the lac repressor which ensures tight control of basal expression from the T7 promoter was subcloned into the pACYC184 vector that has a p15A origin of replication and is compatible with plasmids harboring ColE1 on (FIG. 29B).

In this example, engineered polypeptide referred to as Gpe which sequence originated from lysozyme of Escherichia coli T4 bacteriophage was employed. NCBI Reference Sequence: NP_049736.1, protein name: glycoside hydrolase family protein [Escherichia phage T4]. PelB signal sequence: 1-22, T4 bacteriophage lysozyme 23-164. The protein sequence of Gpe with PelB signal sequence is disclosed herein as SEQ ID NO:1.

### Expression Strain Preparation

One Shot™ BL21 (DE3) E. coli cells were transformed with the pET21-Cas9V2 and pACYC184-PT7-pelB-gpe-lacl plasmids and were plated on LB (animal-origin free) AOF agar with kanamycin (50 g/l) and 25 μg/ml chloramphenicol. Transformations were based on the temperature shock method. A Research cell bank (RCB) was produced cultivating the transformants in liquid TB4 AOF medium with the required antibiotics at 37° C. and 220 rpm. A 50% sterile glycerol solution was used to produce 25% glycerol RCB which were aliquoted in cryovials and stored at −70° C.

### Cultivation Media

Transformations and plasmid propagations were performed on solid and liquid LB AOF medium containing Vegetable peptone (10 g/L), Yeast extract (5 g/l), NaCl (10 g/l), and for solid medium 15 g/l microbiological agar, as well as the required antibiotics. Fed-batch and batch cultivations were performed in glycerol-based animal origin free (AOF) terrific broth (TB4 AOF and TB10 AOF) with the following composition (per litre): Vegetable peptone 12 g, Yeast extract 24 g, (NH₄)₂SO₄ 2.68 g, KH₂PO₄ 2.3 g, K₂HPO₄ 12.5 g and vegetable glycerol 4 to 10 g. Additionally, before cultivation the TB10 medium was supplemented with the following sterile solutions: 3 mL of (1M) MgSO₄ and 3 mL of trace element solution with the following composition (per litre): CaCl₂×2H₂O 0.5 g, ZnSO₄×7H₂O 0.18 g, MnSO₄×H₂O 0.1 g, Na₂-EDTA 20.1 g, FeCl₃×6H₂O 16.7 g, CuSO₄×5H₂O 0.16 g, CoCl₂×6H₂O 0.18 g; as well as 100 μL L-1 of thiamine hydrochloride (1 M), 1 mL 5 of kanamycin (50 mg mL/I) and 1 mL of chloramphenicol (30 mg/l).

### Expression and Autolysis Conditions in Shake Flask

Optimal protein expression medium, IPTG concentration, cell culture incubation time after the induction and the whole protein expression system were determined.

Several different media were tested: BRM medium (20 g/l casein peptone NZ Plus, 10 g/l yeast extract, 5 g/l NaCl, 2.5 g/l K₂HPO₄, 0.4 g/l MgSO₄·7H₂O, 5 g/l glycerol, pH 7) -used for the expression of RUO Cas9; Benzo medium (10 g/l tryptone, 5 g/l yeast extract, 2.68 g/l (NH₄)₂SO4, 1.5 g/l NH₄Cl, 6 g/l KH₂PO₄, 4 g/l K₂HPO₄, 10 g/l glycerol, pH 7) -benchmark medium; TB10 AOF medium (12 g/l vegetable peptone, 24 g/l yeast extract, 2.68 g/l (NH₄)₂SO₄, 2.3 g/l KH₂PO₄, 12.5 g/l K₂HPO₄, 10 g/l glycerol, pH 7.15)—medium used for the expression of other cell banks manufactured under GMP conditions. Cell bank culture was cultivated in these media and induced with 0.5 mM IPTG at OD600=1.6−2.5. Cas9 was expressed at 24° C., since the optimal expression temperature for RUO Cas9 was already determined as 23-25° C., using bacterial host E. coli BL21(DE3). Samples were taken at 4 h and 22 h intervals after the induction and run on the SDS-PAGE gel. The most efficient expression was observed with TB10 AOF cultivation medium. Further, different IPTG concentrations were examined. Cell culture was induced with either 0.1 or 0.5 mM IPTG at OD600=1.88−1.92 and expressed at 24° C. for 22 h. Samples were taken at 4 h and 22 h intervals after the induction and run on the SDS-PAGE gel. Induction with 0.5 mM IPTG resulted in an inhibited growth of the culture after the induction since after 4 h interval it shows more higher protein expression than 0.1 mM IPTG-induced cells, however, after 22 h 0.5 mM induction displays less efficiency. Therefore, the most optimal condition was 0.1 mM IPTG induction (steadier culture growth) for 22 h.

### Expression and Autolysis Conditions in Bioreactor

Preinoculum and inoculum propagations were performed in glycerol-based animal origin free (AOF) terrific broth (TB4 AOF) with the following composition (per litre): Vegetable peptone 12 g, Yeast extract 24 g, (NH₄)₂SO₄ 2.68 g, KH₂PO₄ 2.3 g, K₂HPO₄ 12.5 g and vegetable glycerol 4 g. Additionally, before seeding the TB4 medium was supplemented with the following sterile solutions: 3 mL/L of (1M) MgSO₄ and 3 mL/L of trace element solution (TES) with the following composition (per litre): CaCl₂×2H₂O 0.5 g, ZnSO₄×7H₂O 0.18 g, MnSO₄×H₂O 0.1 g, Na₂-EDTA 20.1 g, FeCl₃×6H₂O 16.7 g, CuSO₄×5H₂O 0.16 g, COCl₂×6H₂O 0.18 g; as well as 3 mL/L of thiamine hydrochloride (10 g/L), 1 mL/L of kanamycin (50 mg mL/I) and 1 mL/L of chloramphenicol (25 mg/l). Terrific broth medium with 15 g glycerol (TB15 AOF) and with same composition except glycerol concentration was used as starting bioreactor media.

### Preinoculum Preparation

A single colony from E. coli BL21(DE3) [pAra-pelB-gpe/pACYC184-PT7-pelB-gpe-lacl] transformants plate was transferred in to 250 mL shake flaks with 50 ml of TB4 AOF medium with supplements and antibiotics and was incubated for 6.5±0.5 h at 37° C./220 rpm. A culture optical density (OD₆₀₀) was measured at the end of cultivation and seed volume for inoculum flask was calculated by formula: 1/OD_{preinoculum}*1000=μL.

### Inoculum Preparation

The calculated preinoculum volume was transferred in to 2000 mL shake flaks with 500 ml of TB4 AOF medium with supplements and antibiotics and was incubated for 17±h at 25° C./180 rpm. A culture optical density (OD₆₀₀) is measured at the end of cultivation and seed volume for bioreactors was calculated by formula: (2000*0.1)/OD_{inoculum}=mL.

### Bioreactor Processes

Batch phase was performed in a 300 L S.U. fermenter (250 L media). Culture growth dynamic was monitored by optical density measurements at 600 nm wavelength. The samples from bioreactors were taken at different process stages and analyzed. The bioreactors seeded with overnight inoculum (initial bioreactor OD 0.07). The initial culture parameters before target protein and simultaneous lysis induction were as follows: the dissolved oxygen (DO) control was maintained at 30% by automatic cascade: air flow (from 125 to 300 L/min); the stirrer rate (from 100 to 375 rpm); pure oxygen flow (from 0 to 266 L/min). Culture pH was controlled at 7.0±0.05 by automatic addition of NH₄OH (25%) or H₃PO₄ (42.5%). The growth temperature before induction was maintained at 37° C. After induction with 0.2 mM IPTG the temperature in all processes was reduced from 37 to 27° C. over 0.5 h time span. 1 h after IPTG induction DO profile was started: 30% to 3% over 0.5h. 2 h after IPTG induction minimum stirrer speed was changed to 100 rpm. IPTG addition simultaneously induce target protein expression and lysozyme expression inside the cells. An induction phase length: 21±1 h. After induction phase gas supply and pH control were stopped, stirrer speed was set to 200 rpm and harvesting/clarification steps were initiated.

### Analysis of Lysis and Expression of Cas9 Protein

Cells and media fractions were separated by centrifugation for 30 min, 14000 rpm, 4° C. Media fractions were stored at 4° C. Cell samples harvested from flask or fermenter cultivations were analyzed by SDS-PAGE electrophoresis. Cell samples were collected at different cultivation times (before induction and after induction). Soluble and insoluble protein fractions were separated by centrifugation for 20 min, 14000 rpm, 4° C. The soluble protein fraction or supernatant after centrifugation represents effectiveness of autolysis process and what part of proteins are soluble. Insoluble fraction represented undisrupted cells and insoluble proteins. Cell samples for SDS-PAGE separation were prepared as follows: 20 μL of crude extract sample, 25 μL of 4×SDS-PAGE loading buffer, 5 μL of 2M DTT and 50 μL of deionized water to obtain a final sample volume of 100 μL. Media samples for SDS-PAGE separation were prepared as follows: 70 μL of culture media, 25 μL of 4×SDS-PAGE loading buffer, 5 μL of 2M to obtain a final sample volume of 100 μL. Samples were heated for 10 min at 95° C. 10 μL of sample was applied to each lane of a 10% SDS-PAGE gel.

### Clarification

The most effective process for Cas9 culture clarification after fermentation is DNA and cell debris sedimentation by 0.15% PEI (polyethylene imine) in high salt concentration (0.1 M Na₂SO₄, 0.4 M NaCl) solution combined with subsequent supernatant depth filtration through P100 media filter.

In more detail, the clarification process involved the following steps (FIG. 36):

Starting material: 250 L of cell lyzed cell culture, after 16-18 h of target protein expression in the stirred tank reactors. Final OD of the cells/cell debris ~15-30 OD. Analysis showed that induced cell lysis resulted in 75-80% cell lysis efficiency from total cell amount.

During all manipulations within stirred tack bioreactor (Hyperforma™ 300 L, Thermofisher Scientific) all automatic controls in fermentation vessel: Air/O2 flow, pH was in “off mode”. The stirring was maintained at 375 rpm and the temperature was set to 12° C.

Enzyme stabilization and flocculation of nucleic acids was carried out as follows:

Dry materials were added directly into the bioreactor vessel, one by one to achieve the final concentration of 0.1M Na₂SO₄ and 0.4 M NaCl. High viscosity solution was mixed thoroughly at 250 rpm for 20 mins at <12° C. After, the stirring was maintained at 100 rpm for 10-18 h at 12 C°. The flocculation was performed by addition of PEI (Lupasol, Polyethyleneimine, BASF) to the bioreactor vessel to achieve final concentration of 0.15% (v/v). Stirring was mixing continued for at least 15 mins at 250 rpm. The flocculation was performed by addition of PEI (Lupasol, Polyethyleneimine, BASF) to the bioreactor vessel to achieve final concentration of 0.15% (v/v). Stirring by mixing continued for at least 15 mins at 250 rpm. The solution was transferred into separate tanks from bioreactor, diluted with water (molecular biology grade) by 2-fold, mixed at 250 rpm for 10-20 mins. After that mixing is stopped completely. Culture was left for sedimentation for ~3 hours at ~10 C°.

Clarification: Filter preparation. The required amount of STAX filter capsules K100P (Pall, ID 7007836) were washed with 10 filter volumes of water. Without exceeding back pressure of 0.2 BAR of the pressure. After sedimentation the culture was clarified by filtering through the depth filtration capsules (1-3 μM (K100) (Pall, ID 7007836) (about 0.05 m² of filter area is needed to filtrate 1 L of fermentation media) without exceeding back pressure of 1.2 BAR. Subsequently clarified solution was filtered through 0.22 μm filter capsule (Pall, ID NP6UEAVP1S). The same clarification process could be performed using filtration systems: 0.4-0.8 μM (K050P (Pall, ID 7007786)).

### Results and Discussion

E. coli is not a perfect host especially for closed single use fermentation process because it normally does not secrete proteins into the extracellular medium. The cell disruption step becomes a limitation and is not cost effective at the pilot or industrial scale when the goal is the implementation of closed and/or continues process flow. The present approach for cell disruption is the programmed cell lysis system based on the expression of a cloned T4 bacteriophage gene gpe to produce a lytic protein. Gene gpe encodes a lysozyme which degrades the peptidoglycan of E. coli cell wall.

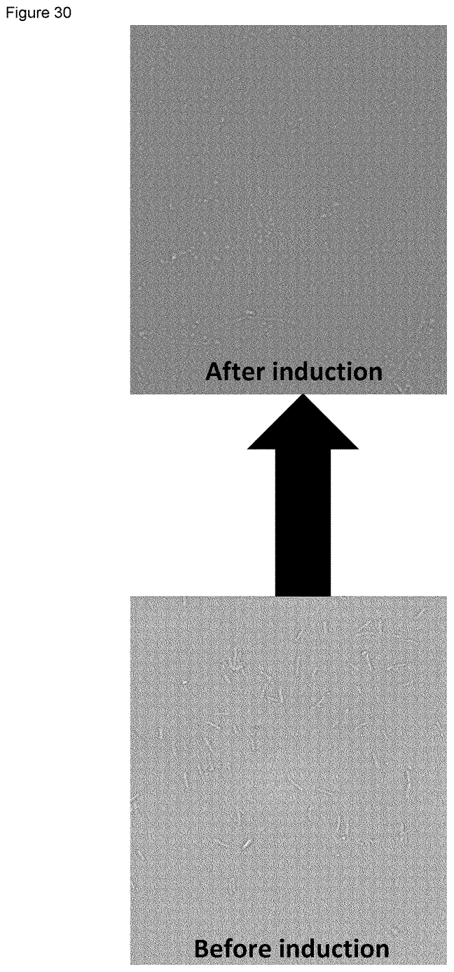

Self-disruptive Escherichia coli that produces Cas9 was developed. E. coli BL21 (DE3) was co-transformed with two vector plasmids a target gene expression vector pET21-Cas9V2 and a lysis gene expression vector pACYC-PT7-pelB-gpe-lacl. The lytic protein was produced after the expression of the target gene, resulting in simplification of the cell disruption process. In this example, the expression of cloned T4 phage gene gpe was used for the disruption of E. coli that produced Cas9 as a target protein. The expression of gene gpe did not lead to prompt cell disruption but weakened the cell wall. The translocation of lytic protein Gpe from the cytoplasm to the periplasm facilitated enzymatic degradation of murein layer which is responsible for maintaining cell strength. Microscopic observation revealed that cells producing Gpe in the periplasmic space after 4 h went through a morphological change, from rod-shaped to elliptical spheroplasts (FIG. 30).

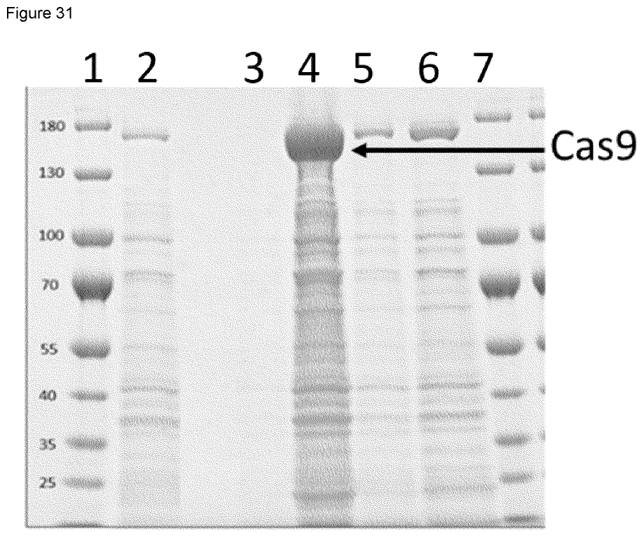

After 22 hour induction 0.1 mM IPTG of E. coli BL21/pET21-Cas9V2/pACYC-PT7-pelB-gpe-lacl Cas9 nuclease was released to the culture medium. Debris of E. coli cells were observed in the culture medium, indicating that cell lysis had occurred. Maximum Cas9 production in shake flask was obtained after overnight expression of E. coli BL21/pET21-Cas9V2/pACYC-PT7-pelB-gpe-lacl Cas9 at 30° C. when lysozyme induction 0.1 mM IPTG was performed together with target gene induction (FIG. 31)

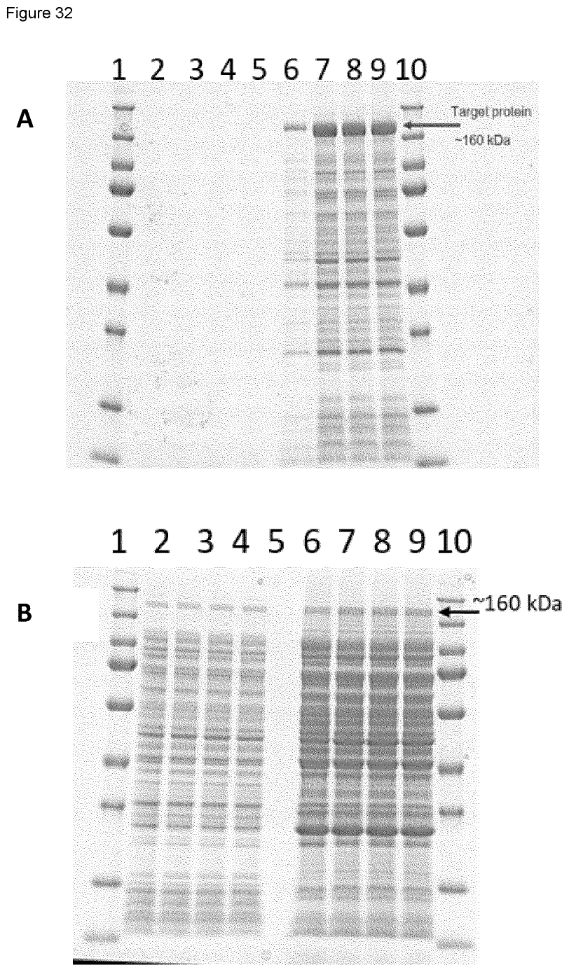

In order to optimize Cas9 expression in BL21/pET21-Cas9V2/pACYC-PT7-pelB-gpe-lacl culture inductor (IPTG) concentration, and induction time were tested. The two different IPTG concentrations: 0.1 and 0.5 mM IPTG, and two different induction time—4 hours and 22 hours were used for Cas9 synthesis induction. The induction of lysozyme gene was performed simultaneously. Culture were grown overnight at 24° C. Samples from inducted culture and media were taken 4 hours and 22 hours after protein synthesis induction. Debris of E. coli cells were observed in the culture medium, indicating that cell lysis had occurred. Maximum Cas9 yield in media was obtained after 22 hours expression/lysis of E. coli JS007/pET21-Cas9V2/pACYC-PT7-pelB-gpe-lacl strain. Cas9 amount was identified by the SDS PAGE (FIG. 32).

FIG. 32A indicated that significant target protein band were seen in samples after induction what means successful expression and autolysis process. FIG. 32B did not show bands of target protein in samples (small lanes that corresponds the size of Cas9 protein −160 kDa which may be the proteins of E. coli cell as the same lanes are seen in samples before induction). Results of analysis of soluble and insoluble fraction indicated that most of non target proteins can be removed during sedimentation and clarification process.

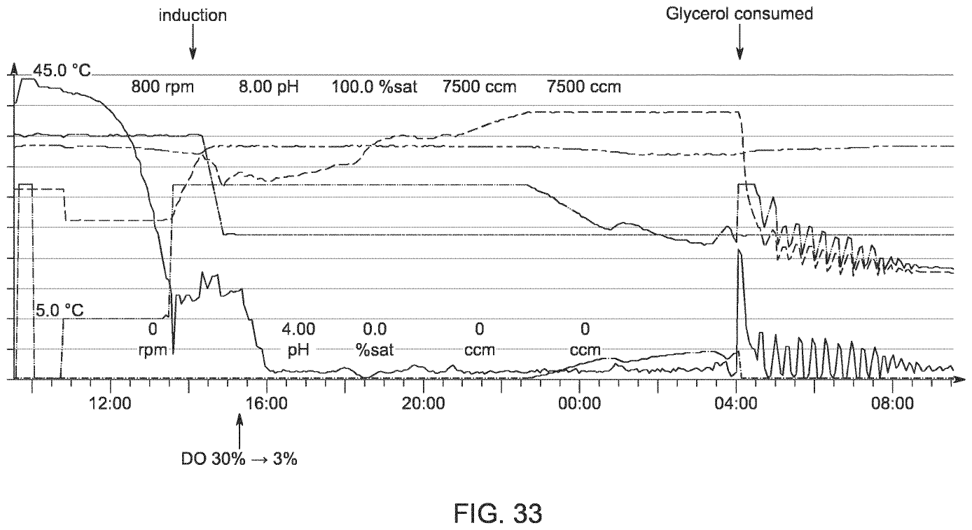

The batch Cas9 production processes in Biostat A stirred bioreactor were designed with the initial medium volume of 3 L. As shown in FIG. 33 the temperature of growth before Cas9 induction (Cas9 induction point: 4±1 OD) was maintained at 37° C. After induction with 0.1 mM IPTG (Cas9 induction point: ±1 OD) the temperature in all processes was reduced from 37 to 30° C. over 0.5 h time span. 1 h after IPTG induction DO profile was started: 30% to 5% over 0.5h. 2 h after IPTG induction minimum stirrer speed was changed to 200 rpm. After induction phase (19.5±1.5 h) gas supply and pH control were stopped, stirrer speed was set to 250 rpm and harvesting/clarification steps were initiated.

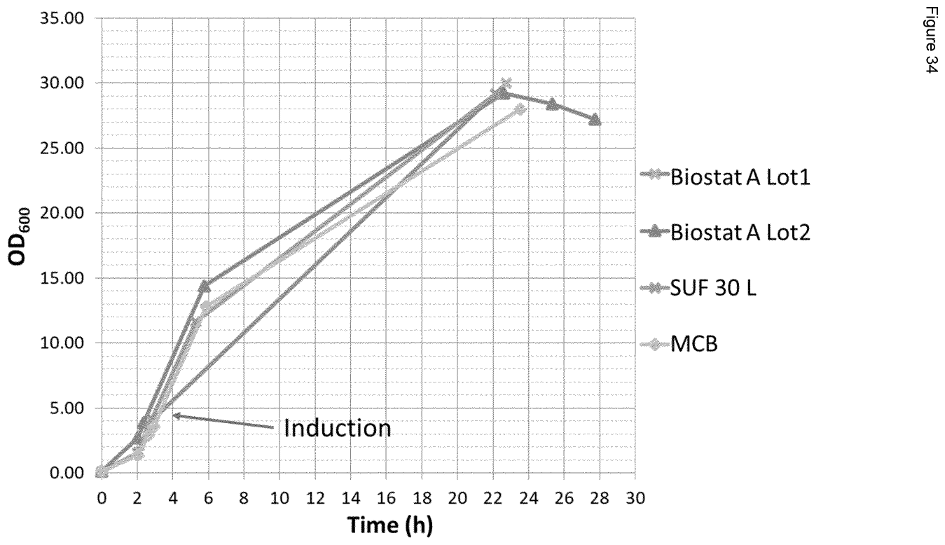

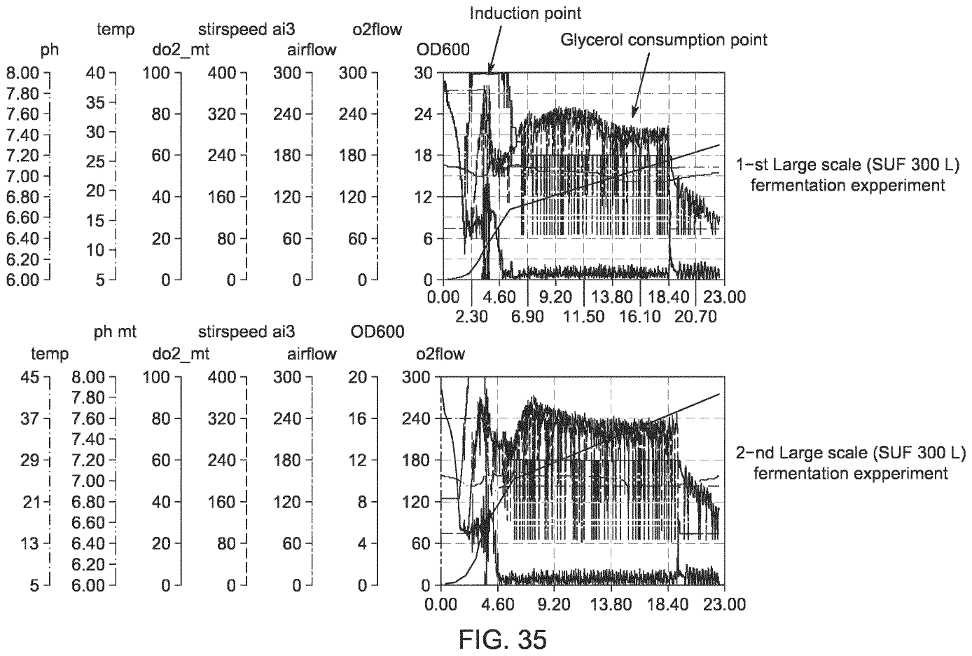

Optimized protein expression conditions were successfully transferred in to 3 L bioreactor (Biostat) FIGS. 34, and 35 and scaled up in 300 L SUF (FIG. 35). After the optimization of fermentation process the yield of Cas9 protein was about 0.3-0.5 g per 1 L of biomass.

Clarification: for optimal cell and cell debris removal from fermentation media final conditions for clarification were: sedimentation step for 0.5-24 hours at 0.15% of PEI and 0.1M Na₂SO₄, 0.4 M NaCl. Following media filtration using 1 m²/30 L K100P STAX depth filter and 0.31 m² 0.22 um XL5 filter. Given clarification conditions ensured effective cell debris removal. After optimized clarification process media was prepared for downstream applications and contains only soluble proteins. Target protein Cas-9 concentration in clarified media was about 0.3-0.5 g/l.

Depth filtration-based clarification of cell debris after nuclease treatment: the blend of high ionic strength creating salts were used to stabilize enzyme and prevent precipitation/aggregation. Also flocculation of nucleic acid was used to create a filtration cake, comprised of small flocculent particles. It was demonstrated successfully using unusually large pores filters. E. coli cell size is: 1.0-2.0 micrometres long, with radius about 0.5 micrometres. Primary clarification approaches which are usually recommended for E. coli are tangential flow filtration clarification <0.2 micrometres pore size or centrifugation, which is the most common approach for clarification of cellular debris, large or small flocculants.

### Example 7—Clarification of Other Recombinant Proteins

### AmpliTaq

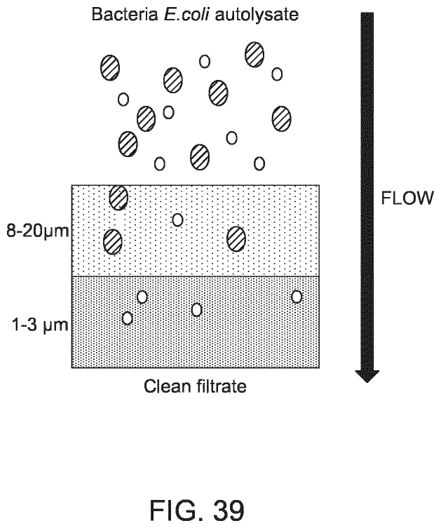

The clarification process for obtaining recombinant Taq DNA polymerase involved the following (FIG. 39):

Starting material: 250 L of cell lyzed cell culture, after 16-18 h of target protein expression in the stirred tank reactors. Final OD of the cells/cell debris ~15-30 OD. Analysis showed that induced cell lysis resulted in 75-80% cell lysis efficiency from total cell amount.

Thermal cellular protein denaturation: during all manipulations within stirred tack bioreactor (Hyperforma™ 300 L, Thermofisher Scientific) all automatic controls in fermentation vessel: Air/O2 flow, pH was in “off mode”. The cell lysate was pumped out of fermenter and passed through the separate coil heating-cooling systems for heating at the ~90° C. and cooling ~20° C. The liquid holding mode-residence time in each coil were maintained for 2 min. The solution of precipitated proteins was collected into the separate tank.

Target protein stabilization and preparation for clarification: stock solutions of 3M KCl and 2 M DTT were added into the volume of cellular protein precipitate to achieve final concentration of 0.6 M and 1 mM, respectively.

Filter preparation: required amount of STAX filter capsule were washed with 10 filter volumes of water (molecular biology grade) without exceeding back pressure of 0.2 BAR.

Clarification: primary clarification through SXLP700416SP filters (Retention 8-20 μm) (about 0.1 m² of filter area was needed to filtrate 1 L of fermentation media).

Secondary clarification: through SXLP100416SP filters (Retention 1-3 μm) (about 0.05 m² of filter area is needed to filtrate 1 L of fermentation media) and subsequently was filtered through 0.22 μm filter capsule (Pall, ID NP6UEAVP1S). All clarifications steps were caried out without exceeding back pressure of 1.2 BAR.

Collected media may be used for further downstream purification steps. The analogical clarification process could be done using clarification alternatives of: K050P (Pall, ID 7007786) Retention 0.4-0.8 μM.

Result and discussion: unique depth example filtration-based clarification of cell debris/denatured protein/nucleic acid slurry after high temperature treatment through unusually large pores filters −8-20 μm. E. coli cell size is: 1.0-2.0 micrometres long, with radius about 0.5 micrometres. Primary clarification approaches which are recommended for E. coli are tangential flow filtration clarification <0.2 micrometres pore size or centrifugation.

Basic Fibroblast Growth Factor (bFGF)

The clarification process for obtaining bFGF involved the following (FIG. 38):

Starting material: 250 L of cell lyzed cell culture, after 16-18 h of target protein expression in the stirred tank reactors. Final OD of the cells/cell debris ~15-30 OD. Analysis showed that induced cell lysis resulted in 75-80% cell lysis efficiency from total cell amount.

During all manipulations within stirred tack bioreactor (Hyperforma™ 300 L, Thermofisher Scientific) all automatic controls in fermentation vessel: Air/O2 flow, pH was in “off mode”. The stirring was maintained at 375 rpm and the temperature was set to 23° C.

Target protein stabilization and removal of nucleic acids: universal nuclease was added into fermentation vessel to achieve final concentration of 250 U/ml. The mixture was incubated for 30-60 min at 23° C. Subsequently, the solution of 5M NaCl solution we added directly to fermentation vessel to achieve final concentration of 0.7 M of NaCl. The mixture of cell debris was mixed at 250 rpm for another 20 mins.

Filter preparation and Clarification: required amount of STAX filter capsule were washed with 10 filter volumes of water (Molecular Biology grade). The back pressure did not exceed 0.2 BAR. The clarification was performed by filtering of suspension through the depth filtration capsules K050P (Pall, ID 7007786) Retention 0.4-0.8 μM (about 0.05 m² of filter area is needed to filtrate 1 L of fermentation media) and subsequent filtration through 0.22 μm filter capsule (Pall, ID NP6UEAVP1S), without exceeding back pressure of 1.2 BAR.

The same clarification process could be performed using filtration capsule combinations:1-3 μM (P100) (Pall, ID 7007836).

Result and discussion: depth filtration-based clarification of cell debris after nuclease treatment—as a result very small particles of membrane segments were generated. It is demonstrated successfully using unusually large pores filters. E. coli cell size is: 1.0-2.0 micrometres long, with radius about 0.5 micrometres. Primary clarification approaches which are recommended for E. coli are tangential flow filtration clarification <0.2 micrometres pore size or centrifugation which is the most common approach for clarification of cellular debris.

### T7 RNA Polymerase

The clarification process for obtaining T7 RNA polymerase involved the following (FIG. 38):

Starting material: 250 L of cell lyzed cell culture, after 16-18 h of target protein expression in the stirred tank reactors. Final OD of the cells/cell debris ~20 OD. Analysis showed that induced cell lysis resulted in 80% cell lysis efficiency from total cell amount.

During all manipulations within stirred tack bioreactor (Hyperforma™ 300 L, Thermofisher Scientific) all automatic controls in fermentation vessel: Air/O2 flow, pH was in “off mode”. The stirring was maintained at 375 rpm and the temperature was set <12° C.

Enzyme stabilization and flocculation of nucleic acids. Dry and liquid materials were added one by one directly to Fermentation vessel to achieve the final concentration of 1M Sucrose, 0.5M Na2SO4 and 0.5% Brij 35. High viscosity solution was mixed thoroughly at 375 rpm for 40 mins <12° C. After, the stirring was maintained at 100 rpm for 10-18h <12 C°. The flocculation was performed by addition of PEI (Lupasol, Polyethyleneimine, BASF) to the bioreactor vessel to achieve final concentration of 0.15% (v/v). Stirring was mixing continued for at least 15 mins at 375 rpm. The solution was transferred into separate tanks from bioreactor, diluted with water (molecular biology grade) by 2.0-fold, mixed at 300 rpm for 20 mins. After that mixing was stopped completely. Culture was left for sedimentation for ~3 hours.

Clarification: Filter preparation. Required amount of STAX filter capsules K050P (Pall, ID 7007786) were washed with 10 filter volumes of water (Molecular biology grade) without exceeding back pressure of 0.2 BAR of the pressure. After sedimentation the upper part of culture was clarified by filtering through the depth filtration capsules K050P (Pall, ID 7007786, retention 0.4-0.8 μm) (about 0.05 m² of filter area is needed to filtrate 1 L of fermentation media) without exceeding back pressure of 1.2 BAR and subsequently is filtered through 0.22 μm filter capsule (Pall, 1-3 ID NP6UEAVP1S). The same clarification process could be performed using filtration systems: 1-3 μM (P100) (Pall, ID 7007836).

Results and discussion: depth filtration-based clarification of cell debris without nuclease treatment. Here, the best blend of high ionic strength creating salts, osmotic agent and detergent were used to stabilize enzyme, prevent precipitation/aggregation, prevent interaction with highly hydrophobic lysozyme and membrane proteins. Also flocculation of nucleic acid was used to create a filtration cake, comprised of small flocculent particles. It is demonstrated successfully using unusually pores of 0.4-08 μm filters. Also it works with 1-3 μm pore filters. E. coli cell size is: 1.0-2.0 micrometres long, with radius about 0.5 micrometres. Primary clarification approaches which are recommended for E. coli are tangential flow filtration clarification <0.2 micrometres pore size or centrifugation, which is the most common approach for clarification of cellular debris, large or small flocculants.

### Example 9—Conclusions

The newly developed expression system disclosed herein comprises genetic elements to produce target proteins and to co-produce lytic enzymes to the cell periplasmic space in the controlled manner at the desired growth period if needed. The above experimental data showed that the co-expressed lytic phage enzyme does not disrupt the cells completely, but rather, forms protoplast like cell units. This suggested that the translocation of lysozyme from the cytoplasm to the periplasm facilitated enzymatic degradation of murein layer, which is responsible for maintaining cell strength. The protoplasts leak/secrete the target proteins to the culture medium without the aid of the secretion leader peptides during shake flasks and microbial bioreactor production. The protoplasts remain viable at least for 4 hours after induction of lytic enzymes, i.e., they are de novo producing and leaking the protein of interest, thus the amounts of the target protein in the culture medium increases over time.

In the case of VTN, protein expression in fermenter (Biostat A or SUF 300 L) was performed in E. coli BL21(DE3) [pET29-optVTN for protein VTN expression and lysis gene expression vector pACYC184-PT7-pelB-gpe-lacl. A means of cell disruption mediated by expression of cloned T4 phage lysis gene is thus shown for the recovery of intracellular target proteins, which can be applied to other recombinant proteins by replacing VTN.

In the case of IPP, expression in Biostat A and 300 L single use bioreactors were performed using E. coli BL21(DE3) [pAra-pelB-gpe]/pET21b-ppa-Kn]cells. Target IPP protein was also found in media in both cases—with cell lysis induction as well in the presents of basal T4 lysozyme expression with its targeted synthesis. So, both types of fermentation (with and without cell lysis induction) could be used in case of IPP production.

A means of cell disruption mediated by expression of cloned T4 phage lysis gene is demonstrated for the recovery of intracellular target proteins, which can be applied and to other recombinant proteins by replacing IPP.

In the case of MuLV-RT, expression in Biostat A fermenter was performed using E. coli JS007 pMuLV-RT-Km, pAra-pelB-gpe cells. A means of cell disruption mediated by expression of cloned T4 phage lysis gene is demonstrated for the recovery of intracellular target proteins, which can be applied to other recombinant proteins.

In the case of RI, expression in Biostat A fermenter was performed using E. coli JS008 pET29-optRNAseOUT, pAra-pelB-gpe cells. A means of cell disruption mediated by expression of cloned T4 phage lysis gene is demonstrated for the recovery of intracellular target proteins, which can be applied to other recombinant proteins by replacing RI.

In the case of OMT, expression in Biostat A fermenter was performed using E. coli JS007 pLATE31-VP39delta32-KnR, pACYC-PT7-pelB-gpe-lacl cells and later processes were upscaled to 300 L single-use fermenter. Up to 70 times higher yield was obtained after clarification and purification processes when compared to expression without lysis. Conventional intercellular OMT expression gives 857 mg and with autolysis—60000 mg from 300 L bioreactor. A means of cell disruption mediated by expression of cloned T4 phage lysis gene is demonstrated for the recovery of intracellular target proteins, which can be applied to other recombinant proteins by replacing OMT.

In the case of cas-9, protein expression in fermenter (Biostat A or SUF 300 L) was performed in E. coli pET21-Cas9V2 target protein Cas-9 and lysis gene expression vector pACYC184-PT7-pelB-gpe-lacl (or pARApeIB-pge). A means of cell disruption mediated by expression of cloned T4 phage lysis gene is demonstrated for the recovery of intracellular target proteins, which can be applied to other recombinant proteins by replacing Cas-9 nuclease.

The disclosed thus demonstrates a one pot process in fermentation vessel of recombinant protein production with cell autolysis followed by protein stabilization and nucleic acid precipitation was employed to enable elimination of at least 5 separately performed subsequent downstream processing steps: cell harvesting, mechanical and or enzymatic cell lysis, cell debris separation, nucleic precipitation followed by separation using filtration or centrifugation.

SEQUENCES
The protein sequence of Gpe with PelB signal sequence
(NCBI Reference Sequence: NP 049736.1, protein name: glycoside
hydrolase family protein (Escherichia phage T4).
PelB signal sequence: 1-22, T4 bacteriophage lysozyme 23-164):
(SEQ ID NO: 1)
MKYLLPTAAAGLLLLAAQPAMAMNIFEMLRIDERLRLKIYKDTEGYYTIGIGHLLTKSPSLNAAKS
ELDKAIGRNCNGVITKDEAEKLENQDVDAAVRGILRNAKLKPVYDSLDAVRRCALINMVFQMGETG
VAGFTNSLRMLQQKRWDEAAVNLAKSIWYNQTPNRAKRVITTERTGTWDAYKNL
The sequence of VTN protein 1-418 (NCBI sequence ID: AAH05046.1)
(SEQ ID NO: 2)
VTRGDVFTMPEDEYTVYDDGEEKNNATVHEQVGGPSLTSDLQAQSKGNPEQTPVLKPEEEAPAPEV
GASKPEGIDSRPETLHPGRPQPPAEEELCSGKPFDAFTDLKNGSLFAFRGQYCYELDEKAVRPGYP
KLIRDVWGIEGPIDAAFTRINCQGKTYLFKGSQYWRFEDGVLDPDYPRNISDGEDGIPDNVDAALA
LPAHSYSGRERVYFFKGKQYWEYQFQHQPSQEECEGSSLSAVFEHFAMMQRDSWEDIFELLEWGRT
SAGTRQPQFISRDWHGVPGQVDAAMAGRIYISGMAPRPSLAKKQRFRHRNRKGYRSQRGHSRGRNQ
NSRRPSRAMWLSLESSEESNLGANNYDDYRMDWLVPATCEPIQSVFFFSGDKYYRVNLRTRRVDTV
DPPYPRSIAQYWLGCPAPGHL
The protein sequence of Inorganic pyrophosphatase 1-287:
(GeneBank: X13253.1, 99% identity, protein sequence:
NCBI Reference Sequence: CAA31629.1)
(SEQ ID NO: 3)
MTYTTRQIGAKNTLEYKVYIEKDGKPVSAFHDIPLYADKENNIFNMVVEIPRWTNAKLEITKEETL
NPIIQDTKKGKLRFVRNCFPHHGYIHNYGAFPQTWEDPNVSHPETKAVGDNDPIDVLEIGETIAYT
GQVKQVKALGIMALLDEGETDWKVIAIDINDPLAPKLNDIEDVEKYFPGLLRATNEWFRIYKIPDG
KPENQFAFSGEAKNKKYALDIIKETHDSWKQLIAGKSSDSKGIDLTNVTLPDTPTYSKAASDAIPP
ASLKADAPIDKSIDKWFFISGSV
The protein sequence of MuLV-RT protein:
(SEQ ID NO: 4)
MTLNIEDEHRLHETSKEPDVSLGSTWLSDFPQAWAETGGMGLAVRQAPLIIPLKATSTPVSIKQYP
MSQEARLGIKPHIQRLLDQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISGQLTWTRLPQGFKNSPT
LFDEALHRDLADERIQHPDLILLQYVDDLLLAATSELDCQQGTRALLQTLGNLGYRASAKKAQICQ
KQVKYLGYLLKEGQRWLTEARKETVMGQPTPKTPRQLREFLGTAGFCRLWIPGFAEMAAPLYPLTK
TGTLFNWGPDQQKAYQEIKQALLTAPALGLPDLTKPFELFVDEKQGYAKGVLTQKLGPWRRPVAYL
SKKLDPVAAGWPPCLRMVAAIAVLTKDAGKLTMGQPLVILAPHAVEALVKQPPDRWLSNARMTHYQ
ALLLDTDRVQFGPVVALNPATLLPLPEEGLQHNCLDILAEAHGTRPDLTDQPLPDADHTWYTDGSS
LLQEGQRKAGAAVTTETEVIWAKALPAGTSAQRAELIALTQALKMAEGKKLNVYTDSRYAFATAHI
HGEIYRRRGLLTSEGKEIKNKDEILALLKALFLPKRLSIIHCPGHQKGHSAEARGNRMADQAARKA
AITENPDTSTLLIENSSPNSRLIN
The protein sequence of Ribonuclease Inhibitor 1-456:
(NCBI GeneID: 445517, protein sequence ID: XP 020938200.1,
protein name: Ribonuclease Inhibitor)
(SEQ ID NO: 5)
MNLDIHCEQLSDARWTELLPLLQQYEVVRLDDCGLTEEHCKDIGSALRANPSLTELCLRTNELGDA
GVHLVLQGLQSPTCKIQKLSLQNCSLTEAGCGVLPSTLRSLPTLRELHLSDNPLGDAGLRLLCEGL
LDPQCHLEKLQLEYCRLTAASCEPLASVLRATRALKELTVSNNDIGEAGARVLGQGLADSACQLET
LRLENCGLTPANCKDLCGIVASQASLRELDLGSNGLGDAGIAELCPGLLSPASRLKTLWLWECDIT
ASGCRDLCRVLQAKETLKELSLAGNKLGDEGARLLCESLLQPGCQLESLWVKSCSLTAACCQHVSL
MLTQNKHLLELQLSSNKLGDSGIQELCQALSQPGTTLRVLCLGDCEVTNSGCSSLASLLLANRSLR
ELDLSNNCVGDPGVLQLLGSLEQPGCALEQLVLYDTYWTEEVEDRLQALEGSKPGLRVIS
The sequence of vp39 protein without 32 C-terminal amino
acids: 1-301, His tag: 302-309: - NCBI Sequence ID: AGJ91263.1,
protein name: multifunctional Poly-A polymerase-
small subunit VP39 [Vaccinia virus]
(SEQ ID NO: 6)
MDVVSLDKPFMYFEEIDNELDYEPESANEVAKKLPYQGQLKLLLGELFFLSKLQRHGILDGATVVY
IGSAPGTHIRYLRDHFYNLGVIIKWMLIDGRHHDPILNGLRDVTLVTRFVDEEYLRSIKKQLHPSK
IILISDVRSKRGGNEPSTADLLSNYALQNVMISILNPVASSLKWRCPFPDQWIKDEYIPHGNKMLQ
PFAPSYSAEMRLLSIYTGENMRLTRVTKSDAVNYEKKMYYLNKIVRNKVVVNEDYPNQEYDYFHMY
FMLRTVYCNKTFPTTKAKVLFLQQSIFRELNIPTTSTGHHHHHHG

1. A method for producing a clarified cell culture comprising a recombinant protein, wherein the method comprises:
i) inoculating a bioreactor with a host cell to provide a cell culture, wherein said host cell is an Escherichia coli transformed with an expression system comprising:
a) a nucleic acid encoding a recombinant protein operably linked to a first inducible promotor; and
b) a nucleic acid encoding a T4 lysozyme protein in frame with a nucleic acid encoding PelB secretory signal peptide operably linked to a second inducible promotor, ii) fermenting the cell culture under conditions that:
a) allow the expression of the recombinant protein; and
b) allow permeabilization of the cell so as to form a spheroplast that secretes the recombinant protein;
iii) clarifying the fermented cell culture comprising the secreted recombinant protein, wherein said clarifying comprises a primary clarification step using a first clarification filter having a pore size that provides a retention range of at least about 0.4 μm or more, wherein the clarification step does not comprise centrifugation.

2. The method of claim 1, wherein the method further comprises a secondary clarification step using a second filter having a pore size that provides a retention range of at least about 0.4 μm or more.

3. The method of claim 1, wherein the primary clarification step and/or secondary clarification step is microfiltration.

4. The method of claim 3, where in the microfiltration is depth filtration.

5. The method of claim 3 wherein the microfiltration is selected from normal flow filtration (NFF) or tangential flow filtration (TFF).

6. The method of claim 1, wherein the primary clarification step uses a first filter having a pore size that provides a retention range of at least about 4.0 to about 9.0 μm, at least about 1.0 to about 3.0 μm, or at least about 0.4 to about 0.8 μm.

7. The method of claim 1, wherein the secondary clarification step uses a second filter having a pore size that provides a retention range of at least about 1.0 to about 3.0 μm or at least about 0.4 to about 0.8 μm.

8. The method of claim 1, wherein the primary clarification step and the secondary clarification step use a filter having the same pore size.

9. The method of claim 1, wherein the clarified cell culture has a turbidity of about less than 20 NTU.

10. The method of claim 1, further comprising a flocculation step prior to the primary clarification step.

11. The method of claim 10, wherein said flocculation step comprises addition of a flocculation agent to the cell culture.

12. The method of claim 11, wherein the flocculation agent is polyethyleneimine (PEI).

13. The method of claim 1, further comprising a nucleic acid inactivation step prior to the primary clarification step.

14. The method of claim 13, wherein said nucleic acid inactivation step comprises addition of a nucleic acid inactivation agent to the cell culture.

15. The method of claim 14, wherein said nucleic acid inactivation agent is benzonase.

16. The method of claim 1, wherein said fermentation step comprises addition of a stabilizing agent to the cell culture.

17. The method of claim 1, wherein the method is performed in a closed system, such that a sterile flow path is maintained between the bioreactor and the first each of the first and second clarification filters.

18. The method of claim 1, further comprising a step of subjecting said clarified cell culture to one or more steps of purification of said recombinant protein.

19. A recombinant protein produced by the method of claim 1.

20.-126. (canceled)
